(19) United States
(12) Patent Application Publication
(10) Pub. No.: US 2008/0080998 A1
(43) Pub. Date: Apr. 3, 2008

(54) Methods for sterilizing tissue

(75) Inventors: Wilson Burgess, Clifton, VA (US); William N. Drohan, Springfield, VA (US); Martin J. MacPhee, Montgomery Village, MD (US); David M. Mann, Gaithersburg, MD (US)

Correspondence Address: MORGAN LEWIS & BOCKIUS LLP, 1111 PENNSYLVANIA AVENUE NW, WASHINGTON, DC, 20004, US

(73) Assignee: Clearant, Inc.

(21) Appl. No.: 11/826,513

(22) Filed: Jul. 16, 2007

Related U.S. Application Data
(63) Continuation of application No. 10/060,208, filed on Feb. 1, 2002, now abandoned.
Application No. 10/060,208 is a continuation-in-part of application No. 09/960,703, filed on Sep. 24, 2001, now abandoned.

(51) Int. Cl. A61L 2/08; A61L 2/00

(52) U.S. Cl. 422/23; 422/22

(57) ABSTRACT

Methods are disclosed for sterilizing tissue to reduce the level of one or more active biological contaminants or pathogens therein, such as viruses, bacteria, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single or multicellular parasites. The methods involve sterilizing one or more tissues with irradiation.

### BACKGROUND OF THE INVENTION

1. Field of the Invention

The present invention relates to methods for sterilizing tissue to reduce the level of one or more active biological contaminants or pathogens therein, such as viruses, bacteria (including inter- and intracellular bacteria, such as mycoplasmas, ureaplasmas, nanobacteria, chlamydia, rickettsias), yeasts, molds, fingi, prions or similar agents responsible, alone or in combination, for transmissible spongiform encephalopathies (TSEs) and/or single or multicellular parasites. The present invention particularly relates to methods of sterilizing tissue with irradiation, wherein the tissue may subsequently be used in transplantation to replace diseased and/or otherwise defective tissue in an animal.

2. Background of the Related Art

Many biological materials that are prepared for human, veterinary, diagnostic and/or experimental use may contain unwanted and potentially dangerous biological contaminants or pathogens, such as viruses, bacteria, in both vegetative and spore states, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single-cell or multicellular parasites. Consequently, it is of utmost importance that any biological contaminant or pathogen in the biological material be inactivated before the product is used. This is especially critical when the material is to be administered directly to a patient, for example in blood transfusions, blood factor replacement therapy, tissue implants, including organ transplants, and other forms of human and/or other animal therapy corrected or treated by surgical implantation, intravenous, intramuscular or other forms of injection or introduction. This is also critical for the various biological materials that are prepared in media or via the culture of cells, or recombinant cells which contain various types of plasma and/or plasma derivatives or other biologic materials and which may be subject to mycoplasmal, prion, ureaplasmal, bacterial, viral and/or other biological contaminants or pathogens.

Recently, the safety of the widespread practice in orthopedic medicine of using human donor tissue to replace damaged cartilage or tendons has come into question. In fact, Federal investigators started looking into the deaths of three patients in Minnesota following knee surgery and found that some people have contracted severe infections after receiving implanted knee tissue, which appeared to be infected with a type of bacteria, known as Clostridium. Maura Lerner, et al, “Knee Surgery Deaths Turn Focus on Donor Tissue”, Star Tribune, Dec. 8, 2001. See also “Septic Arthritis Following Anterior Cruciate Ligament Reconstruction Using Tendon Allografts—Florida and Louisiana, 2000”, MMWR Weekly, 50(48):1081-1083 (Dec. 7, 2001).

The tissue in these knee surgery cases was cartilage, which is not sterilized as it is believed such sterilization would damage the implant. Instead, tissue suppliers attempt to provide safe tissue through screening donors, testing for bacteria and applying antibiotic solutions. In fact, many procedures for producing human compatible biological materials have involved methods that screen or test the biological materials for one or more particular biological contaminants or pathogens rather than removal or inactivation of the contaminant(s) or pathogen(s) from the biological material. The typical protocol for disposition of materials that test positive for a biological contaminant or pathogen simply is non-use/discarding of that material. In certain cases, known microbial contaminants may be permitted in the implant material at the time it is harvested from the host organism. Examples of screening procedures for contaminants include testing for a particular virus in human blood and tissues from donors. Such procedures, however, are not always reliable, as evidenced by the death of at least one Minnesota man who received a cartilage implant, and are not able to detect the presence of prions or certain viruses, particularly those present in very low numbers. This reduces the value, certainty, and safety of such tests in view of the consequences associated with a false negative result, which can be life threatening in certain cases, for example in the case of Acquired Immune Deficiency Syndrome (AIDS). Furthermore, in some instances it can take weeks, if not months, to determine whether or not the material is contaminated. Moreover, to date, there is no commercially available, reliable test or assay for identifying prions, ureaplasmas, mycoplasmas, and chlamydia within a biological material that is fully suitable for screening out potential donors or infected material (Advances in Contraception 10(4):309-315 (1994)). This serves to heighten the need for an effective means of destroying prions, ureaplasmas, mycoplasmas, chlamydia, etc., within a biological material, while still retaining the desired activity of that material. Therefore, it would be desirable to apply techniques that would kill or inactivate contaminants or pathogens during and/or after manufacturing and/or harvesting the biological material.

The importance of ready availability of effective techniques is apparent regardless of the source of the biological material. All living cells and multi-cellular organisms can be infected with viruses and other pathogens. Thus, the products of unicellular natural or recombinant organisms or tissues virtually always carry a risk of pathogen contamination. In addition to the risk that the producing cells or cell cultures may be infected, the processing of these and other biological materials also creates opportunities for environmental contamination. The risks of infection are more apparent for multicellular natural and recombinant organisms, such as transgenic animals. Interestingly, even products from species as different from humans as transgenic plants carry risks, both due to processing contamination as described above, and from environmental contamination in the growing facilities, which may be contaminated by pathogens from the environment or infected organisms that co-inhabit the facility along with the desired plants. For example, a crop of transgenic corn grown out doors, could be expected to be exposed to rodents such as mice during the growing season. Mice can harbor serious human pathogens such as the frequently fatal Hanta virus. Since these animals would be undetectable in the growing crop, viruses shed by the animals could be carried into the transgenic material at harvest. Indeed, such rodents are notoriously difficult to control, and may gain access to a crop during sowing, growth, harvest or storage. Likewise, contamination from overflying or perching birds has the potential to transmit such serious pathogens as the causative agent for psittacosis. Thus, any biological material, regardless of its source, may harbor serious pathogens that must be removed or inactivated prior to administration of the material to a recipient human or other animal.

In conducting experiments to determine the ability of technologies to inactivate viruses, the actual viruses of concern are seldom utilized. This is a result of safety concerns for the workers conducting the tests, and the difficulty and expense associated with facilities for containment and waste disposal. In their place, model viruses of the same family and class are usually used. In general, it is acknowledged that the most difficult viruses to inactivate are those with an outer shell made up of proteins, and that among these, the most difficult to inactivate are those of the smallest size. This has been shown to be true for gamma irradiation and most other forms of radiation because these viruses' diminutive size is associated with a small genome. The magnitude of direct effects of radiation upon a molecule is directly proportional to the size of the molecule; that is, the larger the target molecule, the greater is the effect. As a corollary, it has been shown for gamma-irradiation that the smaller the viral genome, the higher is the radiation dose required to inactive it.

Among the viruses of concern for both human and animal-derived biological materials, the smallest, and thus most difficult to inactivate, belong to the family of Parvoviruses and the slightly larger protein-coated Hepatitis virus. In humans, the Parvovirus B19, and Hepatitis A are the agents of concern. In porcine-derived materials, the smallest corresponding virus is Porcine Parvovirus. Since this virus is harmless to humans, it is frequently chosen as a model virus for the human B19 Parvovirus. The demonstration of inactivation of this model parvovirus is considered adequate proof that the method employed will kill human B19 virus and Hepatitis A, and, by extension, that it will also kill the larger and less hardy viruses, such as HIV, CMV, Hepatitis B, Hepatitis C, and others.

More recent efforts have focussed on methods to remove or inactivate contaminants in products intended for use in humans and other animals. Such methods include heat treating, filtration and the addition of chemical inactivants or sensitizers to the product.

According to current standards of the U.S. Food and Drug Administration, heat treatment of biological materials may require heating to approximately 60° C. for a minimum of 10 hours, which can be damaging to sensitive biological materials. Indeed, heat inactivation can destroy 50% or more of the biological activity of certain biological materials. Tissues are particularly sensitive to these high temperature treatments.

Filtration involves filtering the product in order to physically remove contaminants. Unfortunately, this method may also remove products that have a high molecular weight. Further, in certain cases, small viruses may not be removed by the filter.

The procedure of chemical sensitization involves the addition of noxious agents which bind to the DNA/RNA of the virus, and which are activated either by UV or other radiation. This radiation produces reactive intermediates and/or free radicals which bind to the DNA/RNA of the virus, break the chemical bonds in the backbone of the DNA/RNA, and/or cross-link or complex it in such a way that the virus can no longer replicate. This procedure requires that unbound sensitizer be washed from products since the sensitizers are toxic, if not mutagenic or carcinogenic, and cannot be administered to a patient.

Irradiating a product with gamma radiation is another method of sterilizing a product. Gamma radiation is effective in destroying viruses and bacteria when given in high total doses (Keathly, et al., “Is There Life After Irradiation? Part 2,” BioPharm July-August, 1993, and Leitman, “Use of Blood Cell Irradiation in the Prevention of Post Transfusion Graft-vs-Host Disease,” Transfusion Science 10:219-239 (1989)). The published literature in this area, however, teaches that gamma radiation can be damaging to radiation sensitive products, such as blood, blood products, protein and protein-containing products. In particular, it has been shown that high radiation doses are injurious to red cells, platelets and granulocytes (Leitman). U.S. Pat. No. 4,620,908 discloses that protein products must be frozen prior to irradiation in order to maintain the viability of the protein product. This patent concludes that “[i]f the gamma irradiation were applied while the protein material was at, for example, ambient temperature, the material would be also completely destroyed, that is the activity of the material would be rendered so low as to be virtually ineffective.” Unfortunately, many sensitive biological materials, such as monoclonal antibodies (Mab), may lose viability and activity if subjected to freezing for irradiation purposes and then thawing prior to administration to a patient.

When the product to be sterilized is biological tissue that is to be transplanted, even greater sensitivity to irradiation or other sterilization method is often encountered. This greater sensitivity is the result of the molecular integration of the biochemical, physiological, and anatomical systems that is required for normal function of that biological tissue. Thus, special procedures are typically required to maintain the tight molecular integration that underpins normal function during and after transplantation of a biological tissue. Furthermore, special procedures may be required in addition to other considerations, such as histocompatibility (matching of HLA types, etc.) between donor and recipient, and including compatibility between species when there is inter-species (i.e., heterografting) transplantation.

Tissues and organs that may be used in transplantation are numerous. Non-limiting examples include heart, lung, liver, spleen, pancreas, kidney, corneas, bone, joints, bone marrow, blood cells (red blood cells, leucocytes, lymphocytes, platelets, etc.), plasma, skin, fat, tendons, ligaments, hair, muscles, blood vessels (arteries, veins), teeth, gum tissue, fetuses, eggs (fertilized and not fertilized), eye lenses, and even hands. Active research may soon expand this list to permit transplantation of nerve cells, nerves, and other physiologically and anatomically complex tissues, including intestine, cartilage, entire limbs, and portions of brain.

As surgical techniques become more sophisticated, and as storage and preparation techniques improve, the demand for various kinds of transplantation may reasonably be expected to increase over current levels.

Another factor that may feed future transplantation demand is certain poor lifestyle choices in the population, including such factors as poor nutrition (including such trends as the increasing reliance on so-called fast foods and fried foods; insufficient intake of fruits, vegetables and true whole grains; and increased intake of high glycemic, low nutritional value foods, including pastas, breads, white rice, crackers, potato chips and other snack foods, etc.), predilections toward a sedentary lifestyle, and over-exposure to ultraviolet light in tanning booths and to sunlight. The increasing occurrence of such factors as these have resulted, for example, in increased incidences of obesity (which also exacerbates such conditions as arthritis and conditions with cartilage damage, as well as impairs wound healing, immune function, cancer risk, etc.), type II diabetes and polycystic ovary syndrome (high post prandial glucose values causing damage to such tissues as nerve, muscle, kidney, heart, liver, etc., causing tissue and organ damage even in persons who are not diabetic), many cancers, and hypertension and other cardiovascular conditions, such as strokes and Alzheimer's disease (recent data suggesting that Alzheimer's may be the result of a series of mini-strokes). Thus, poor lifestyle choices ultimately will increase demand for bone, cartilage, skin, blood vessels, nerves, and the specific tissues and organs so destroyed or damaged.

Infections comprise yet another factor in transplantation demand. Not only can bacterial and viral infections broadly damage the infected host tissue or organ, but they can also spread vascularly or by lymphatics to cause lymph vessel or vascular inflammation, and/or plaque build up that ultimately results in infarct (for example, stroke, heart attack, damaged or dead tissue in lung or other organ, etc.). In addition, there is an epidemic of infection by intracellular microbes for which reliable commercial tests are not available (for example, mycoplasma, ureaplasma, and chlamydia), for example, as a result of sexual contact, coughing, etc. [for example, more than 20% of sore throats in children are due to chlamydia (E. Normann, et al., “Chlamydia Pneumoniae in Children Undergoing Adenoidectomy,” Acta Paediatrica 90(2): 126-129 (2001))].

Some intravascular infectious agents, via the antibodies that are produced to fight them, result in attack of tissue having surface molecules that have a molecular structure similar to the structure of surface or other groups of the infectious agent. Such is the case with some Streptococci infections (antibodies produced against M proteins of Streptococci that cross-react with cardiac, joint and other tissues), for example, in which tissue and other cardiac tissue may be attacked to cause reduced cardiac function, and which can result in death if the infection is not properly treated before extensive damage occurs. Another antibody mediated condition that can affect cardiac tissue, among other tissues/cells, is antiphospholipid antibody syndrome (APLA), in which antibodies are directed against certain phospholipids (cardiolipin) to produce a hypercoagulable state, thrombocytopenia, fetal loss, dementia, strokes, optic changes, Addison's disease, and skin rashes, among other symptoms. Tissue vegetations and mitral regurgitation are common in intravascular infections, although tissue destruction so extensive as to require valve replacement is rare.

Other intravascular infectious agents directly attack tissues and organs in/on which they establish colonies. Non-limiting examples include Staphylococci (including, for example, S. aureus, S. epidermidis, S. saprophyticus, among others), Chlamydia (including, for example, C. pneumoniae, among others), Streptococci (including, for example, the viridians group of Streptococci: S. sanguis, S. oralis (mitis), S. salivarius, S. mutans, and others; and other species of Streptococci, such as S. bovis and S. pyogenes), Enterococci (for example, E. faecalis and E. faecium, among others), various fungi, and the “HACEK” group of gram-negative bacilli (Haemophilus parainfluenzae, Haemophilus aphrophilus, Actinibacillus actnomycetemcomitans, Cardiobacterium hominis, Eikenella corrodens, and Kingella kingae), Neisseria gonorrhoeae, Clostridia sp., Listeria moncytogenes, Salmonella sp., Bacteroides fragilis, Escherichia coli, Proteus sp., mycoplasmas, ureaplasmas, various viruses (for example, cytomegalovirus, HIV, and herpes simplex virus), and Klebsiella-Enterobacter-Serratia sp., among others.

An exemplary study by Nystrom-Rosander, et al. may be cited for showing the presence of Chlamydia pneumoniae in sclerotic tissue that required replacement as a result of the sclerosis. (C. Nystrom-Rosander, et al., “High Incidence of Chlamydia pneumoniae in Sclerotic Tissue of Patients Undergoing Aortic Valve Replacement” Scandinavian Journal of Infectious Disease 29:361-365 (1997).

Yet another factor in transplantation demand is drug use, particularly the use of illicit drugs, but also including inappropriate and sometimes illegal use of otherwise licit drugs (such as overuse of alcohol/alcoholism causing cirrhosis of the liver, and therefore requiring liver transplantation). Such drug use often strongly damages or even destroys sensitive tissues and organs such as kidney, liver, lung, heart, brain/nerves, and/or portions thereof. In addition, intravenous drug use greatly increases the odds of contracting intravascular infections by any one or more of the above-cited infectious agents (among many others), which infections can attack virtually any organ or portion thereof, including the tricuspid valve (located between the right atrium and the right ventricle), the mitral valve (located between the left atrium and the left ventricle), the pulmonary or pulmonic valve (located between the right ventricle and the pulmonary artery), and the aortic valve (located between the left ventricle and the aorta) with any infectious agent that may enter through implanted tissue.

In view of the difficulties discussed above, there remains a need for methods of sterilizing biological materials that are effective for reducing the level of active biological contaminants or pathogens without an adverse effect on the material(s).

The above references are incorporated by reference herein where appropriate for appropriate teachings of additional or alternative details, features and/or technical background.

### SUMMARY OF THE INVENTION

An object of the invention is to solve at least the related art problems and disadvantages, and to provide at least the advantages described hereinafter.

Accordingly, it is an object of the present invention to provide methods of sterilizing tissue by reducing the level of active biological contaminants or pathogens without adversely affecting the tissue or other material. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a method for sterilizing one or more tissues that are sensitive to radiation, the method comprising irradiating the one or more tissues with radiation for a time effective to sterilize the one or more tissues at a rate effective to sterilize the one or more tissues and to protect the one or more tissues from the radiation.

Another embodiment of the present invention is directed to a method for sterilizing one or more tissues that are sensitive to radiation, comprising: (i) applying to the one or more tissues at least one stabilizing process selected from the group consisting of: (a) adding to the one or more tissues at least one stabilizer in an amount effective to protect the one or more tissues from the radiation; (b) reducing the residual solvent content of the one or more tissues to a level effective to protect the one or more tissues from the radiation; (c) reducing the temperature of the one or more tissues to a level effective to protect the one or more tissues from the radiation; (d) reducing the oxygen content of the one or more tissues to a level effective to protect the one or more tissues from the radiation; (e) adjusting or maintaining the pH of the one or more tissues to a level effective to protect the one or more tissues from the radiation; and (f) adding to the one or more tissues at least one non-aqueous solvent in an amount effective to protect the one or more tissues from the radiation; and (ii) irradiating the one or more tissues with a suitable radiation at an effective rate for a time effective to sterilize the one or more tissues.

Another embodiment of the present invention is directed to a method for sterilizing one or more tissues that are sensitive to radiation, comprising: (i) applying to the one or more tissues at least one stabilizing process selected from the group consisting of: (a) adding to the one or more tissues at least one stabilizer; (b) reducing the residual solvent content of the one or more tissues; (c) reducing the temperature of the one or more tissues; (d) reducing the oxygen content of the one or more tissues; (e) adjusting or maintaining the pH of the one or more tissues; and (f) adding to the one or more tissues at least one non-aqueous solvent; and (ii) irradiating the one or more tissues with a suitable radiation at an effective rate for a time effective to sterilize the one or more tissues, wherein the at least one stabilizing process and the rate of irradiation are together effective to protect the one or more tissues from the radiation.

Another embodiment of the present invention is directed to a method for sterilizing one or more tissues that are sensitive to radiation, comprising: (i) applying to the one or more tissues at least two stabilizing processes selected from the group consisting of: (a) adding to the one or more tissues at least one stabilizer; (b) reducing the residual solvent content of the one or more tissues; (c) reducing the temperature of the one or more tissues; (d) reducing the oxygen content of the one or more tissues; (e) adjusting or maintaining the pH of the one or more tissues; and (f) adding to the one or more tissues at least one non-aqueous solvent; and (ii) irradiating the one or more tissues with a suitable radiation at an effective rate for a time effective to sterilize the one or more tissues, wherein the at least two stabilizing processes are together effective to protect the one or more tissues from the radiation and further wherein the at least two stabilizing processes may be performed in any order.

Another embodiment of the present invention is directed to methods for sterilizing one or more tissues that are sensitive to radiation while producing substantially no neo-antigens in the tissue and/or reducing the number of reactive allo-antigens and/or xeno-antigens. Such methods reduce post-implantation complications including, but not limited to, inflammation, immune rejection reactions, calcification, and similar conditions that reduce the implant's ability to function and/or survive in the recipient.

Another embodiment of the present invention is directed to methods for prophylaxis or treatment of a condition or disease or malfunction of a tissue in a mammal comprising introducing into a mammal in need thereof one or more tissues sterilized according to the methods above.

Another embodiment of the present invention is directed to a composition comprising one or more tissues and at least one stabilizer in an amount effective to preserve the one or more tissues for their intended use following sterilization with radiation.

Another embodiment of the present invention is directed to a composition comprising one or more tissues, wherein the residual solvent content of the one or more tissues is at a level effective to preserve the one or more tissues for their intended use following sterilization with radiation.

Another embodiment of the present invention is directed to an assay for determining the optimal conditions for sterilizing a tissue other than collagen without adversely affective a predetermined biological characteristic or property thereof, comprising the steps of: (i) irradiating collagen under a pre-determined set of conditions effective to sterilize tissue; (ii) determining the turbidity of the irradiated collagen; and (iii) repeating steps (i) and (ii) with a different pre-determined set of conditions until the turbidity of the collagen reaches a pre-determined acceptable level.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

FIGS. 1A-1D show the effects of gamma irradiation on porcine heart valves in the presence of polypropylene glycol 400 and, optionally, a scavenger.

FIGS. 2A-2E show the effects of gamma irradiation on porcine heart valve cusps in the presence of 50% DMSO and, optionally, a stabilizer, and in the presence of polypropylene glycol 400.

FIGS. 3A-3E show the effects of gamma irradiation on frozen porcine AV heart valves soaked in various solvents and irradiated to a total dose of 30 kGy at 1.584 kGy/hr at −20° C.

FIGS. 4A-4H show the effects of gamma irradiation on frozen porcine AV heart valves soaked in various solvent and irradiated to a total dose of 45 kGy at approximately 6 kGy/hr at −70° C.

FIGS. 5A-5E show the effects of gamma irradiation on frozen porcine ACL tissue soaked in a stabilizer cocktail and irradiated to a total dose of 45 kGy at approximately. 6 kGy/hr at −80° C.

FIGS. 6A-6F show the effects of gamma irradiation on frozen porcine ACL tissue soaked in the various stabilizers.

FIGS. 7A-7C show the effects of gamma irradiation on frozen porcine ACL tissue soaked in cryopreservatives using either regulated freeze or quick freeze.

FIG. 8 shows the effects of a combination of ethanol dehydration or drying to remove water and rehydration to deliver a stabilizer cocktail to frozen porcine ACL tissue to protect the samples from gamma irradiation to a total dose of 50 kGy at 4° C.

FIGS. 9A-9B show the effects of salts and pH levels on scavengers inside ACL tissue to protect the ACL tissue from gamma irradiation to a total dose of 50 kGy at −80° C.

FIG. 10 shows the effects of gamma irradiation on frozen porcine ACL tissue soaked in various alcohols and irradiated to a total dose of 50 kGy at −80° C.

FIG. 11 shows the effects of gamma irradiation on fresh frozen, freeze-dried or solvent dried porcine ACL tissue irradiated to a total dose of 45 kGy at about −72° C.

FIGS. 12A-12C show the effects of gamma irradiation on type I collagen treated with various stabilizers and irradiated to a total dose of 45 kGy at −20° C., -80° C. or freeze dried at 4° C.

FIG. 13 shows the effects of gamma irradiation on liquid and gel collagen treated with various stabilizers.

FIGS. 14A-14D show the effects of gamma irradiation on collagen treated with various stabilizers.

### DETAILED DESCRIPTION OF PREFERRED EMBODIMENTS

### A. Definitions

Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates otherwise.

As used herein, the term “sterilize” is intended to mean a reduction in the level of at least one active biological contaminant or pathogen found in the tissue being treated according to the present invention.

As used herein, the term “non-aqueous solvent” is intended to mean any liquid other than water in which a biological material, such as one or more tissues, may be dissolved or suspended or which may be disposed within a biological material, such as one or more tissues, and includes both inorganic solvents and, more preferably, organic solvents. Illustrative examples of suitable non-aqueous solvents include, but are not limited to, the following: alkanes and cycloalkanes, such as pentane, 2-methylbutane (isopentane), heptane, hexane, cyclopentane and cyclohexane; alcohols, such as methanol, ethanol, 2-methoxyethanol, isopropanol, n-butanol, t-butyl alcohol, and octanol; esters, such as ethyl acetate, 2-methoxyethyl acetate, butyl acetate and benzyl benzoate; aromatics, such as benzene, toluene, pyridine, xylene; ethers, such as diethyl ether, 2-ethoxyethyl ether, ethylene glycol dimethyl ether and methyl t-butyl ether; aldehydes, such as formaldehyde and glutaraldehyde; ketones, such as acetone and 3-pentanone (diethyl ketone); glycols, including both monomeric glycols, such as ethylene glycol and propylene glycol, and polymeric glycols, such as polyethylene glycol (PEG) and polypropylene glycol (PPG), e.g., PPG 400, PPG 1200 and PPG 2000; acids and acid anhydrides, such as formic acid, acetic acid, trifluoroacetic acid, phosphoric acid and acetic anhydride; oils, such as cottonseed oil, peanut oil, culture media, polyethylene glycol, poppyseed oil, safflower oil, sesame oil, soybean oil and vegetable oil; amines and amides, such as piperidine, N,N-dimethylacetamide and N,N-deimethylformamide; dimethylsulfoxide (DMSO); nitriles, such as benzonitrile and acetonitrile; hydrazine; detergents, such as polyoxyethylenesorbitan monolaurate (Tween 20) and monooleate (Tween 80), Triton and sodium dodecyl sulfate; carbon disulfide; halogenated solvents, such as dichloromethane, chloroform, carbon tetrachloride, 1,2-dichlorobenzene, 1,2-dichloroethane, tetrachloroethylene and 1-chlorobutane; furans, such as tetrahydrofuran; oxanes, such as 1,4-dioxane; and glycerin/glycerol. Particularly preferred examples of suitable non-aqueous solvents include non-aqueous solvents which also function as stabilizers, such as ethanol and acetone.

As used herein, the term “biological contaminant or pathogen” is intended to mean a biological contaminant or pathogen that, upon direct or indirect contact with a biological material, such as one or more tissues, may have a deleterious effect on the biological material or upon a recipient thereof. Such other biological contaminants or pathogens include the various viruses, bacteria, in both vegetative and spore states, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single or multicellular parasites known to those of skill in the art to generally be found in or infect biological materials. Examples of other biological contaminants or pathogens include, but are not limited to, the following: viruses, such as human immunodeficiency viruses and other retroviruses, herpes viruses, filoviruses, circoviruses, paramyxoviruses, cytomegaloviruses, hepatitis viruses (including hepatitis A, B, C, and D variants thereof, among others), pox viruses, toga viruses, Ebstein-Barr viruses and parvoviruses; bacteria, such as Escherichia, Bacillus, Campylobacter, Streptococcus and Staphylococcus; nanobacteria; parasites, such as Trypanosoma and malarial parasites, including Plasmodium species; yeasts; molds; fungi; mycoplasmas and ureaplasmas; chlamydia; rickettsias, such as Coxiella burnetti; and prions and similar agents responsible, alone or in combination, for one or more of the disease states known as transmissible spongiform encephalopathies (TSEs) in mammals, such as scrapie, transmissible mink encephalopathy, chronic wasting disease (generally observed in mule deer and elk), feline spongiform encephalopathy, bovine spongiform encephalopathy (mad cow disease), Creutzfeld-Jakob disease (including variant CJD), Fatal Familial Insomnia, Gerstinann-Straeussler-Scheinker syndrome, kuru and Alpers syndrome. As used herein, the term “active biological contaminant or pathogen” is intended to mean a biological contaminant or pathogen that is capable of causing a deleterious effect, either alone or in combination with another factor, such as a second biological contaminant or pathogen or a native protein (wild-type or mutant) or antibody, in a biological material, such as one or more tissues, and/or a recipient thereof

As used herein, the term “a biologically compatible solution” is intended to mean a solution to which a biological material, such as one or more tissues, may be exposed, such as by being suspended or dissolved therein, and retain its essential biological and physiological characteristics. Such solutions may be of any suitable pH, tonicity, concentration and/or ionic strength.

As used herein, the term “a biologically compatible buffered solution” is intended to mean a biologically compatible solution having a pH and osmotic properties (e.g., tonicity, osmolality and/or oncotic pressure) suitable for maintaining the integrity of the material(s) therein, such as one or more tissues. Suitable biologically compatible buffered solutions typically have a pH between 2 and 8.5 and are isotonic or only moderately hypotonic or hypertonic. Biologically compatible buffered solutions are known and readily available to those of skill in the art. Greater or lesser pH and/or tonicity may also be used in certain applications. The ionic strength of the solution may be high or low, but is typically similar to the environments in which the tissue is intended to be used.

As used herein, the term “stabilizer” is intended to mean a compound or material that, alone and/or in combination, reduces damage to the biological material being irradiated to a level that is insufficient to preclude the safe and effective use of the material. Illustrative examples of stabilizers that are suitable for use include, but are not limited to, the following, including structural analogs and derivatives thereof: antioxidants; free radical scavengers, including spin traps, such as tert-butyl-nitrosobutane (tNB), a-phenyl-tert-butylnitrone (PBN), 5,5-dimethylpyrroline-N-oxide (DMPO), tert-butylnitrosobenzene (BNB), a-(4-pyridyl-1-oxide)-N-tert-butylnitrone (4-POBN) and 3,5-dibromo-4-nitroso-benzenesulphonic acid (DBNBS); combination stabilizers, i.e., stabilizers which are effective at quenching both Type I and Type II photodynamic reactions; and ligands, ligand analogs, substrates, substrate analogs, modulators, modulator analogs, stereoisomers, inhibitors, and inhibitor analogs, such as heparin, that stabilize the molecule(s) to which they bind. Preferred examples of additional stabilizers include, but are not limited to, the following: fatty acids, including 6,8-dimercapto-octanoic acid (lipoic acid) and its derivatives and analogues (alpha, beta, dihydro, bisno and tetranor lipoic acid), thioctic acid, 6,8-dimercapto-octanoic acid, dihydrolopoate (DL-6,8-dithioloctanoic acid methyl ester), lipoamide, bisonor methyl ester and tetranor-dihydrolipoic acid, omega-3 fatty acids, omega-6 fatty acids, omega-9 fatty acids, furan fatty acids, oleic, linoleic, linolenic, arachidonic, eicosapentaenoic (EPA), docosahexaenoic (DHA), and palmitic acids and their salts and derivatives; carotenes, including alpha-, beta-, and gamma-carotenes; Co-Q10; xanthophylls; sucrose, polyhydric alcohols, such as glycerol, mannitol, inositol, and sorbitol; sugars, including derivatives and stereoisomers thereof, such as xylose, glucose, ribose, mannose, fructose, erythrose, threose, idose, arabinose, lyxose, galactose, allose, altrose, gulose, talose, and trehalose; amino acids and derivatives thereof, including both D- and L-forms and mixtures thereof, such as arginine, lysine, alanine, valine, leucine, isoleucine, proline, phenylalanine, glycine, serine, threonine, tyrosine, asparagine, glutamine, aspartic acid, histidine, N-acetylcysteine (NAC), glutamic acid, tryptophan, sodium capryl N-acetyl tryptophan, and methionine; azides, such as sodium azide; enzymes, such as Superoxide Dismutase (SOD), Catalase, and Δ4, Δ5 and Δ6 desaturases; uric acid and its derivatives, such as 1,3-dimethyluric acid and dimethylthiourea; allopurinol; thiols, such as glutathione and reduced glutathione and cysteine; trace elements, such as selenium, chromium, and boron; vitamins, including their precursors and derivatives, such as vitamin A, vitamin C (including its derivatives and salts such as sodium ascorbate and palmitoyl ascorbic acid) and vitamin E (and its derivatives and salts such as alpha-, beta-, gamma-, delta-, epsilon-, zeta-, and eta-tocopherols, tocopherol acetate and alpha-tocotrienol); chromanol-alpha-C6; 6-hydroxy-2,5,7,8-tetramethylchroma-2 carboxylic acid (Trolox) and derivatives; extraneous proteins, such as gelatin and albumin; tris-3-methyl-1-phenyl-2-pyrazolin-5-one (MCI-186); citiolone; puercetin; chrysin; dimethyl sulfoxide (DMSO); piperazine diethanesulfonic acid (PIPES); imidazole; methoxypsoralen (MOPS); 1,2-dithiane-4,5-diol; reducing substances, such as butylated hydroxyanisole (BHA) and butylated hydroxytoluene (BHT); cholesterol, including derivatives and its various oxidized and reduced forms thereof, such as low density lipoprotein (LDL), high density lipoprotein (HDL), and very low density lipoprotein (VLDL); probucol; indole derivatives; thimerosal; lazaroid and tirilazad mesylate; proanthenols; proanthocyanidins; ammonium sulfate; Pegorgotein (PEG-SOD); N-tert-butyl-alpha-phenylnitrone (PBN); 4-hydroxy-2,2,6,6-tetramethylpiperidin-1-oxyl (Tempol); mixtures of ascorbate, urate and Trolox C (Asc/urate/Trolox C); proteins, such as albumin, and peptides of two or more amino acids, any of which may be either naturally occurring amino acids, i.e., L-amino acids, or non-naturally occurring amino acids, i.e., D-amino acids, and mixtures, derivatives, and analogs thereof, including, but not limited to, arginine, lysine, alanine, valine, leucine, isoleucine, proline, phenylalanine, glycine, histidine, glutamic acid, tryptophan (Trp), serine, threonine, tyrosine, asparagine, glutamine, aspartic acid, cysteine, methionine, and derivatives thereof, such as N-acetylcysteine (NAC) and sodium capryl N-acetyl tryptophan, as well as homologous dipeptide stabilizers (composed of two identical amino acids), including such naturally occurring amino acids, as Gly-Gly (glycylglycine) and Trp-Trp, and heterologous dipeptide stabilizers (composed of different amino acids), such as arnosine (β-alanyl-histidine), anserine (β-alanyl-methylhistidine), and Gly-Trp; and flavonoids/flavonols, such as diosmin, quercetin, rutin, silybin, silidianin, silicristin, silymarin, apigenin, apiin, chrysin, morin, isoflavone, flavoxate, gossypetin, myricetin, biacalein, kaempferol, curcumin, proanthocyanidin B2-3-O-gallate, epicatechin gallate, epigallocatechin gallate, epigallocatechin, gallic acid, epicatechin, dihydroquercetin, quercetin chalcone, 4,4′-dihydroxy-chalcone, isoliquiritigenin, phloretin, coumestrol, 4′,7-dihydroxy-flavanone, 4′,5-dihydroxy-flavone, 4′,6-dihydroxy-flavone, luteolin, galangin, equol, biochanin A, daidzein, formononetin, genistein, amentoflavone, bilobetin, taxifolin, delphinidin, malvidin, petunidin, pelargonidin, malonylapiin, pinosylvin, 3-methoxyapigenin, leucodelphinidin, dihydrokaempferol, apigenin 7-O-glucoside, pycnogenol, aminoflavone, purpogallin fisetin, 2′,3′-dihydroxyflavone, 3-hydroxyflavone, 3′,4′-dihydroxyflavone, catechin, 7-flavonoxyacetic acid ethyl ester, catechin, hesperidin, and naringin. Particularly preferred examples include single stabilizers or combinations of stabilizers that are effective at quenching both Type I and Type II photodynamic reactions, and volatile stabilizers, which can be applied as a gas and/or easily removed by evaporation, low pressure, and similar methods. Additional preferred examples for use in the methods of the present invention include hydrophobic stabilizers.

As used herein, the term “residual solvent content” is intended to mean the amount or proportion of freely-available liquid in the biological material. Freely-available liquid means the liquid, such as water and/or an organic solvent (e.g., ethanol, isopropanol, polyethylene glycol, etc.), present in the biological material being sterilized that is not bound to or complexed with one or more of the non-liquid components of the biological material. Freely-available liquid includes intracellular water and/or other solvents. The residual solvent contents related as water referenced herein refer to levels determined by the FDA approved, modified Karl Fischer method (Meyer and Boyd, Analytical Chem., 31:215-219, 1959; May, et al., J. Biol. Standardization, 10:249-259, 1982; Centers for Biologics Evaluation and Research, FDA, Docket No. 89D-0140, 83-93; 1990) or by near infrared spectroscopy. Quantitation of the residual levels of water or other solvents may be determined by means well known in the art, depending upon which solvent is employed. The proportion of residual solvent to solute may also be considered to be a reflection of the concentration of the solute within the solvent. When so expressed, the greater the concentration of the solute, the lower the amount of residual solvent.

As used herein, the term “sensitizer” is intended to mean a substance that selectively targets viruses, bacteria, in both vegetative and spore states, (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs, rendering them more sensitive to inactivation by radiation, therefore permitting the use of a lower rate or dose of radiation and/or a shorter time of irradiation than in the absence of the sensitizer. Illustrative examples of suitable sensitizers include, but are not limited to, the following: psoralen and its derivatives and analogs (including 3-carboethoxy psoralens); inactines and their derivatives and analogs; angelicins, khellins and coumarins which contain a halogen substituent and a water solubilization moiety, such as quaternary ammonium ion or phosphonium ion; nucleic acid binding compounds; brominated hematoporphyrin; phthalocyanines; purpurins; porphyrins; halogenated or metal atom-substituted derivatives of dihematoporphyrin esters, hematoporphyrin derivatives, benzoporphyrin derivatives, hydrodibenzoporphyrin dimaleimade, hydrodibenzoporphyrin, dicyano disulfone, tetracarbethoxy hydrodibenzoporphyrin, and tetracarbethoxy hydrodibenzoporphyrin dipropionamide; doxorubicin and daunomycin, which may be modified with halogens or metal atoms; netropsin; BD peptide, S2 peptide; S-303 (ALE compound); dyes, such as hypericin, methylene blue, eosin, fluoresceins (and their derivatives), flavins, merocyanine 540; photoactive compounds, such as bergapten; and SE peptide. In addition, atoms which bind to prions, and thereby increase their sensitivity to inactivation by radiation, may also be used. An illustrative example of such an atom would be the Copper ion, which binds to the prion protein and, with a Z number higher than the other atoms in the protein, increases the probability that the prion protein will absorb energy during irradiation, particularly gamma irradiation.

As used herein, the term “radiation” is intended to mean radiation of sufficient energy to sterilize at least some component of the irradiated biological material. Types of radiation include, but are not limited to, the following: (i) corpuscular (streams of subatomic particles such as neutrons, electrons, and/or protons); (ii) electromagnetic (originating in a varying electromagnetic field, such as radio waves, visible (both mono and polychromatic) and invisible light, infrared, ultraviolet radiation, x-radiation, and gamma rays and mixtures thereof); and (iii) sound and pressure waves. Such radiation is often described as either ionizing (capable of producing ions in irradiated materials) radiation, such as gamma rays, and non-ionizing radiation, such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of Cobalt or Cesium, while UV and X-rays are produced by machines that emit UV and X-radiation, respectively, and electrons are often used to sterilize materials in a method known as “E-beam” irradiation that involves their production via a machine. Visible light, both mono- and polychromatic, is produced by machines and may, in practice, be combined with invisible light, such as infrared and UV, that is produced by the same machine or a different machine.

As used herein, the term “tissue” is intended to mean a substance derived or obtained from a multi-cellular living organism that performs one or more functions in the organism or a recipient thereof. Thus, as used herein, a “tissue” may be an aggregation of intercellular substance(s), such as collagen, elastin, fibronectin, fibrin, glycosaminoglycans and the like, and/or cells which are generally morphologically similar, such as hemapoietic cells, bone cells and the like. Accordingly, the term “tissue” is intended to include both allogenic and autologous tissue, including, but not limited to, cellular viable tissue, cellular non-viable tissue and acellular tissue, such as collagen, elastin, fibronectin, fibrin, glycosaminoglycans and the like. As used herein, the term “tissue” includes naturally occurring tissues, such as tissues removed from a living organism and used as such, or processed tissues, such as tissue processed so as to be less antigenic, for example allogenic tissue intended for transplantation, and tissue processed to allow cells to proliferate into the tissue, for example demineralised bone matrix that has been processed to enable bone cells to proliferate into and through it or heart valves that have been processed to encourage cell engraftment following implantation. Additionally, as used herein, the term “tissue” is intended to include natural, artificial, synthetic, semi-synthetic or semi-artificial materials comprised of biomolecules structured in such a way as to permit the replacement of at least some function(s) of a natural tissue when implanted into a recipient. Such constructs may be placed in a cell-containing environment prior to implantation to encourage their cellularization. Illustrative examples of tissues that may be treated according to the methods of the present invention include, but are not limited to, the following: connective tissue; epithelial tissue; adipose tissue; cartilage, bone (including demineralised bone matrix); muscle tissue; and nervous tissue. Non-limiting examples of specific tissues that may be treated according to the methods of the present invention include heart, lung, liver, spleen, pancreas, kidney, corneas, joints, bone marrow, blood cells (red blood cells, leucocytes, lymphocytes, platelets, etc.), plasma, skin, fat, tendons, ligaments, hair, muscles, blood vessels (arteries, veins), teeth, gum tissue, fetuses, eggs (fertilized and not fertilized), eye lenses, hands, nerve cells, nerves, and other physiologically and anatomically complex tissues, such as intestine, cartilage, entire limbs, cadavers, and portions of brain, and intracellular substances, such as collagen, elastin, fibrinogen, fibrin, fibronectin, glycosaminoglycans, and polysaccharides.

As used herein, the term “to protect” is intended to mean to reduce any damage to the biological material, such as one or more tissues, being irradiated, that would otherwise result from the irradiation of that material, to a level that is insufficient to preclude the safe and effective use of the material following irradiation. In other words, a substance or process “protects” a biological material, such as one or more tissues, from radiation if the presence of that substance or carrying out that process results in less damage to the material from irradiation than in the absence of that substance or process. Thus, a biological material, such as one or more tissues, may be used safely and effectively after irradiation in the presence of a substance or following performance of a process that “protects” the material, but could not be used with as great a degree of safety or as effectively after irradiation under identical conditions but in the absence of that substance or the performance of that process.

As used herein, an “acceptable level” of damage may vary depending upon certain features of the particular method(s) of the present invention being employed, such as the nature and characteristics of the particular one or more tissues and/or non-aqueous solvent(s) being used, and/or the intended use of the material being irradiated, and can be determined empirically by one skilled in the art. An “unacceptable level” of damage would therefore be a level of damage that would preclude the safe and effective use of the biological material, such as one or more tissues, being sterilized. The particular level of damage in a given biological material may be determined using any of the methods and techniques known to one skilled in the art.

### B. Particularly Preferred Embodiments

A first preferred embodiment of the present invention is directed to a method for sterilizing one or more tissues that are sensitive to radiation, the method comprising irradiating the one or more tissues with radiation for a time effective to sterilize the one or more tissues at a rate effective to sterilize the one or more tissues and to protect the one or more tissues from the radiation.

A second preferred embodiment of the present invention is directed to a method for sterilizing one or more tissues that are sensitive to radiation, comprising: (i) applying to the one or more tissues at least one stabilizing process selected from the group consisting of: (a) adding to the one or more tissues at least one stabilizer in an amount effective to protect the one or more tissues from the radiation; (b) reducing the residual solvent content of the one or more tissues to a level effective to protect the one or more tissues from the radiation; (c) reducing the temperature of the one or more tissues to a level effective to protect the one or more tissues from the radiation; (d) reducing the oxygen content of the one or more tissues to a level effective to protect the one or more tissues from the radiation; (e) adjusting or maintaining the pH of the one or more tissues to a level effective to protect the one or more tissues from the radiation; and (f) adding to the one or more tissues at least one non-aqueous solvent in an amount effective to protect the one or more tissues from the radiation; and (ii) irradiating the one or more tissues with a suitable radiation at an effective rate for a time effective to sterilize the one or more tissues.

A third preferred embodiment of the present invention is directed to a method for sterilizing one or more tissues that are sensitive to radiation, comprising: (i) applying to the one or more tissues at least one stabilizing process selected from the group consisting of: (a) adding to the one or more tissues at least one stabilizer; (b) reducing the residual solvent content of the one or more tissues; (c) reducing the temperature of the one or more tissues; (d) reducing the oxygen content of the one or more tissues; (e) adjusting or maintaining the pH of the one or more tissues; and (f) adding to the one or more tissues at least one non-aqueous solvent; and (ii) irradiating the one or more tissues with a suitable radiation at an effective rate for a time effective to sterilize the one or more tissues, wherein the at least one stabilizing process and the rate of irradiation are together effective to protect the one or more tissues from the radiation.

A fourth preferred embodiment of the present invention is directed to a method for sterilizing one or more tissues that are sensitive to radiation, comprising: (i) applying to the one or more tissues at least two stabilizing processes selected from the group consisting of: (a) adding to the one or more tissues at least one stabilizer; (b) reducing the residual solvent content of the one or more tissues; (c) reducing the temperature of the one or more tissues; (d) reducing the oxygen content of the one or more tissues; (e) adjusting or maintaining the pH of the one or more tissues; and (f) adding to the one or more tissues at least one non-aqueous solvent; and (ii) irradiating the one or more tissues with a suitable radiation at an effective rate for a time effective to sterilize the one or more tissues, wherein the at least two stabilizing processes are together effective to protect the one or more tissues from the radiation and further wherein the at least two stabilizing processes may be performed in any order.

Another preferred embodiment of the present invention is directed to a composition comprising one or more tissues and at least one stabilizer in an amount effective to preserve the one or more tissues for their intended use following sterilization with radiation.

Another preferred embodiment of the present invention is directed to a composition comprising one or more tissues, wherein the residual solvent content of the one or more tissues is at a level effective to preserve the one or more tissues for their intended use following sterilization with radiation.

Another preferred embodiment of the present invention is directed to a composition comprising one or more tissues, at least one non-aqueous solvent and at least one stabilizer in an amount effective to preserve the one or more tissues for their intended use following sterilization with radiation.

A composition comprising one or more tissues and at least one stabilizer, wherein the residual solvent content of the one or more tissues is at a level that together with the at least one stabilizer is effective to preserve the one or more tissues for their intended use following sterilization with radiation.

The non-aqueous solvent is preferably a non-aqueous solvent that is not prone to the formation of free-radicals upon irradiation, and more preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

According to certain embodiments of the present invention, the one or more tissues may contain a mixture of water and a non-aqueous solvent, such as ethanol and/or acetone. In such embodiments, the non-aqueous solvent(s) is (are) preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are also stabilizers, such as ethanol and acetone.

According to certain methods of the present invention, a stabilizer is added prior to irradiation of the one or more tissues with radiation. This stabilizer is preferably added to the one or more tissues in an amount that is effective to protect the one or more tissues from the radiation. Alternatively, the stabilizer is added to the one or more tissues in an amount that, together with a non-aqueous solvent, is effective to protect the one or more tissues from the radiation. Suitable amounts of stabilizer may vary depending upon certain features of the particular method(s) of the present invention being employed, such as the particular stabilizer being used and/or the nature and characteristics of the particular one or more tissues being irradiated and/or its intended use, and can be determined empirically by one skilled in the art.

According to certain methods of the present invention, the residual solvent content of the one or more tissues is reduced prior to irradiation of the one or more tissues with radiation. The residual solvent content is preferably reduced to a level that is effective to protect the one or more tissues from the radiation. Suitable levels of residual solvent content may vary depending upon certain features of the particular method(s) of the present invention being employed, such as the nature and characteristics of the particular one or more tissues being irradiated and/or its intended use, and can be determined empirically by one skilled in the art. There may be tissue for which it is desirable to maintain the residual solvent content to within a particular range, rather than a specific value.

According to certain embodiments of the present invention, when the one or more tissues also contain water, the residual solvent (water) content of one or more tissues may be reduced by dissolving or suspending the one or more tissues in a non-aqueous solvent that is capable of dissolving water. Preferably, such a non-aqueous solvent is not prone to the formation of free-radicals upon irradiation and has little or no dissolved oxygen or other gas(es) that is (are) prone to the formation of free-radicals upon irradiation.

While not wishing to be bound by any theory of operability, it is believed that the reduction in residual solvent content reduces the degrees of freedom of the one or more tissues, reduces the number of targets for free radical generation and may restrict the diffusability of these free radicals. Similar results might therefore be achieved by lowering the temperature of the one or more tissues below their eutectic point(s) or below their freezing point(s), or by vitrification to likewise reduce the degrees of freedom of the one or more tissues. These results may permit the use of a higher rate and/or dose of radiation than might otherwise be acceptable. Thus, the methods described herein may be performed at any temperature that doesn't result in unacceptable damage to the one or more tissues, i.e., damage that would preclude the safe and effective use of the one or more tissues. Preferably, the methods described herein are performed at ambient temperature or below ambient temperature, such as below the eutectic point(s) or freezing point(s) of the one or more tissues being irradiated.

In certain embodiments of the present invention, the desired residual solvent content of a particular tissue may be found to lie within a range, rather than at a specific point. Such a range for the preferred residual solvent content of a particular tissue may be determined empirically by one skilled in the art.

The residual solvent content of the one or more tissues may be reduced by any of the methods and techniques known to those skilled in the art for reducing solvent from one or more tissues without producing an unacceptable level of damage to the one or more tissues. Such methods include, but are not limited to, lyophilization, drying, concentration, addition of alternative solvents, evaporation, chemical extraction and vitrification.

A particularly preferred method for reducing the residual solvent content of one or more tissues is lyophilization.

Another particularly preferred method for reducing the residual solvent content of one or more tissues is vitrification, which may be accomplished by any of the methods and techniques known to those skilled in the art, including the addition of solute and or additional solutes, such as sucrose, to raise the eutectic point(s) of the one or more tissues, followed by a gradual application of reduced pressure to the one or more tissues in order to remove the residual solvent. The resulting glassy material will then have a reduced residual solvent content.

According to certain methods of the present invention, the one or more tissues to be sterilized may be immobilized upon or attached to a solid surface by any means known and available to one skilled in the art. For example, the one or more tissues to be sterilized may be attached to a biological or non-biological substrate.

The radiation employed in the methods of the present invention may be any radiation effective for the sterilization of the one or more tissues being treated. The radiation may be corpuscular, including E-beam radiation. Preferably the radiation is electromagnetic radiation, including x-rays, infrared, visible light, UV light and mixtures of various wavelengths of electromagnetic radiation. A particularly preferred form of radiation is gamma radiation.

According to the methods of the present invention, the one or more tissues are irradiated with the radiation at a rate effective for the sterilization of the one or more tissues, while not producing an unacceptable level of damage to the one or more tissues. Suitable rates of irradiation may vary depending upon certain features of the methods of the present invention being employed, such as the nature and characteristics of the particular tissue, which may contain a non-aqueous solvent, being irradiated, the particular form of radiation involved, and/or the particular biological contaminants or pathogens being inactivated. Suitable rates of irradiation can be determined empirically by one skilled in the art. Preferably, the rate of irradiation is constant for the duration of the sterilization procedure. When this is impractical or otherwise not desired, a variable or discontinuous irradiation may be utilized.

According to the methods of the present invention, the rate of irradiation may be optimized to produce the most advantageous combination of product recovery and time required to complete the operation. Both low (≦3 kGy/hour) and high (>3 kGy/hour) rates may be utilized in the methods described herein to achieve such results. The rate of irradiation is preferably selected to optimize the recovery of the one or more tissues while still sterilizing the one or more tissues. Although reducing the rate of irradiation may serve to decrease damage to the one or more tissues, it will also result in longer irradiation times being required to achieve a particular desired total dose. A higher dose rate may therefore be preferred in certain circumstances, such as to minimize logistical issues and costs, and may be possible particularly when used in accordance with the methods described herein for protecting tissue from irradiation.

According to a particularly preferred embodiment of the present invention, the rate of irradiation is not more than about 3.0 kGy/hour, more preferably between about 0.1 kGy/hr and 3.0 kGy/hr, even more preferably between about 0.25 kGy/hr and 2.0 kGy/hour, still even more preferably between about 0.5 kGy/hr and 1.5 kGy/hr and most preferably between about 0.5 kGy/hr and 1.0 kGy/hr.

According to another particularly preferred embodiment of the present invention, the rate of irradiation is at least about 3.0 kGy/hr, more preferably at least about 6 kGy/hr, even more preferably at least about 16 kGy/hr, even more preferably at least about 30 kGy/hr and most preferably at least about 45 kGy/hr or greater.

According to the methods of the present invention, the one or more tissues to be sterilized are irradiated with the radiation for a time effective for the sterilization of the one or more tissues. Combined with irradiation rate, the appropriate irradiation time results in the appropriate dose of irradiation being applied to the one or more tissues. Suitable irradiation times may vary depending upon the particular form and rate of radiation involved and/or the nature and characteristics of the particular one or more tissues being irradiated. Suitable irradiation times can be determined empirically by one skilled in the art.

According to the methods of the present invention, the one or more tissues to be sterilized are irradiated with radiation up to a total dose effective for the sterilization of the one or more tissues, while not producing an unacceptable level of damage to those one or more tissues. Suitable total doses of radiation may vary depending upon certain features of the methods of the present invention being employed, such as the nature and characteristics of the particular one or more tissues being irradiated, the particular form of radiation involved, and/or the particular biological contaminants or pathogens being inactivated. Suitable total doses of radiation can be determined empirically by one skilled in the art. Preferably, the total dose of radiation is at least 25 kGy, more preferably at least 45 kGy, even more preferably at least 75 kGy, and still more preferably at least 100 kGy or greater, such as 150 kGy or 200 kGy or greater.

The particular geometry of the one or more tissues being irradiated, such as the thickness and distance from the source of radiation, may be determined empirically by one skilled in the art. A preferred embodiment is a geometry that provides for an even rate of irradiation throughout the preparation of one or more tissues. A particularly preferred embodiment is a geometry that results in a short path length for the radiation through the preparation, thus minimizing the differences in radiation dose between the front and back of the preparation. This may be further minimized in some preferred geometries, particularly those wherein the preparation of one or more tissues has a relatively constant radius about its axis that is perpendicular to the radiation source and by the utilization of a means of rotating the preparation of one or more tissues about said axis.

Similarly, according to certain methods of the present invention, an effective package for containing the preparation of one or more tissues during irradiation is one which combines stability under the influence of irradiation, and which minimizes the interactions between the package of one or more tissues and the radiation. Preferred packages maintain a seal against the external environment before, during and post-irradiation, and are not reactive with the preparation of one or more tissues within, nor do they produce chemicals that may interact with the preparation of one or more tissues within. Particularly preferred examples include but are not limited to containers that comprise glasses stable when irradiated, stoppered with stoppers made of rubber or other suitable materials that is relatively stable during radiation and liberates a minimal amount of compounds from within, and sealed with metal crimp seals of aluminum or other suitable materials with relatively low Z numbers. Suitable materials can be determined by measuring their physical performance, and the amount and type of reactive leachable compounds post-irradiation, and by examining other characteristics known to be important to the containment of such biological materials as tissue empirically by one skilled in the art.

According to certain methods of the present invention, an effective amount of at least one sensitizing compound may optionally be added to the one or more tissues prior to irradiation, for example to enhance the effect of the irradiation on the biological contaminant(s) or pathogen(s) therein, while employing the methods described herein to minimize the deleterious effects of irradiation upon the one or more tissues. Suitable sensitizers are known to those skilled in the art, and include psoralens and their derivatives and inactines and their derivatives.

According to the methods of the present invention, the irradiation of the one or more tissues may occur at any temperature that is not deleterious to the one or more tissues being sterilized. According to one preferred embodiment, the one or more tissues are irradiated at ambient temperature. According to an alternate preferred embodiment, the one or more tissues are irradiated at reduced temperature, i.e., a temperature below ambient temperature, such as 0° C., −20° C., −40° C., −60° C., −78° C. or −196° C. According to this embodiment of the present invention, the one or more tissues are preferably irradiated at or below the freezing or eutectic point(s) of the one or more tissues or the residual solvent therein. According to another alternate preferred embodiment, the one or more tissues are irradiated at elevated temperature, i.e., a temperature above ambient temperature, such as 37° C., 60° C., 72° C. or 80° C. While not wishing to be bound by any theory, the use of elevated temperature may enhance the effect of irradiation on the biological contaminant(s) or pathogen(s) and therefore allow the use of a lower total dose of radiation.

Most preferably, the irradiation of the one or more tissues occurs at a temperature that protects the preparation of one or more tissues from radiation. Suitable temperatures can be determined empirically by one skilled in the art.

In certain embodiments of the present invention, the temperature at which irradiation is performed may be found to lie within a range, rather than at a specific point. Such a range for the preferred temperature for the irradiation of a particular tissue may be determined empirically by one skilled in the art.

According to the methods of the present invention, the irradiation of the one or more tissues may occur at any pressure which is not deleterious to the one or more tissues being sterilized. According to one preferred embodiment, the one or more tissues are irradiated at elevated pressure. More preferably, the one or more tissues are irradiated at elevated pressure due to the application of sound waves or the use of a volatile. While not wishing to be bound by any theory, the use of elevated pressure may enhance the effect of irradiation on the biological contaminant(s) or pathogen(s) and/or enhance the protection afforded by one or more stabilizers, and therefore allow the use of a lower total dose of radiation. Suitable pressures can be determined empirically by one skilled in the art.

Generally, according to the methods of the present invention, the pH of the one or more tissues undergoing sterilization is about 7. In some embodiments of the present invention, however, the one or more tissues may have a pH of less than 7, preferably less than or equal to 6, more preferably less than or equal to 5, even more preferably less than or equal to 4, and most preferably less than or equal to 3. In alternative embodiments of the present invention, the one or more tissues may have a pH of greater than 7, preferably greater than or equal to 8, more preferably greater than or equal to 9, even more preferably greater than or equal to 10, and most preferably greater than or equal to 11. According to certain embodiments of the present invention, the pH of the preparation of one or more tissues undergoing sterilization is at or near the isoelectric point of one of the components of the one or more tissues. Suitable pH levels can be determined empirically by one skilled in the art.

Similarly, according to the methods of the present invention, the irradiation of the one or more tissues may occur under any atmosphere that is not deleterious to the one or more tissues being treated. According to one preferred embodiment, the one or more tissues are held in a low oxygen atmosphere or an inert atmosphere. When an inert atmosphere is employed, the atmosphere is preferably composed of a noble gas, such as helium or argon, more preferably a higher molecular weight noble gas, and most preferably argon. According to another preferred embodiment, the one or more tissues are held under vacuum while being irradiated. According to a particularly preferred embodiment of the present invention, the one or more tissues (lyophilized, liquid or frozen) are stored under vacuum or an inert atmosphere (preferably a noble gas, such as helium or argon, more preferably a higher molecular weight noble gas, and most preferably argon) prior to irradiation. According to an alternative preferred embodiment of the present invention, the one or more tissues are held under low pressure, to decrease the amount of gas, particularly oxygen and nitrogen, dissolved in the liquid, prior to irradiation, either with or without a prior step of solvent reduction, such as lyophilization. Such degassing may be performed using any of the methods known to one skilled in the art. For example, the one or more tissues may be treated prior to irradiation with at least one cycle, and preferably three cycles, of being subjected to a vacuum and then being placed under an atmosphere comprising at least one noble gas, such as argon, or nitrogen.

In another preferred embodiment, where the one or more tissues contain oxygen or other gases dissolved within the one or more tissues or within their container or associated with them, the amount of these gases within or associated with the preparation of one or more tissues may be reduced by any of the methods and techniques known and available to those skilled in the art, such as the controlled reduction of pressure within a container (rigid or flexible) holding the preparation of one or more tissues to be treated or by placing the preparation of one or more tissues in a container of approximately equal volume.

In certain embodiments of the present invention, when the one or more tissues to be treated contains an aqueous or non-aqueous solvent, or a mixture of such solvents, at least one stabilizer is introduced according to any of the methods and techniques known and available to one skilled in the art, including soaking the tissue in a solution containing the stabilizer(s), preferably under pressure, at elevated temperature and/or in the presence of a penetration enhancer, such as dimethylsulfoxide, and more preferably, when the stabilizer(s) is a protein, at a high concentration. Other methods of introducing at least one stabilizer into tissue include, but are not limited to, the following: applying a gas containing the stabilizer(s), preferably under pressure and/or at elevated temperature; injecting the stabilizer(s) or a solution containing the stabilizer(s) directly into the tissue; placing the tissue under reduced pressure and then introducing a gas or solution containing the stabilizer(s); dehydrating the tissue, such as by using a buffer of high ionic and/or osmolar strength, and rehydrating the tissue with a solution containing the stabilizer(s); applying a high ionic strength solvent containing the stabilizer(s), which may optionally be followed by a controlled reduction in the ionic strength of the solvent; cycling the tissue between solutions of high ionic and/or osmolar strength and solutions of low ionic and/or osmolar strength containing the stabilizer(s); and combinations of two or more of these methods. One or more sensitizers may also be introduced into tissue according to such methods.

According to certain embodiments of the present invention, in order to enhance penetration of one or more stabilizers and/or sensitizers into the tissue, one or more compounds effective to increase penetration into the tissue may be employed. For instance, the tissue may treated with one or more compounds that cause an increase in the distance between molecules in the tissue, thereby promoting penetration of the stabilizers and/or sensitizers into the tissue.

Similarly, the tissue may be treated with one or more compounds that cause macromolecules in the tissue to become less compact, or relaxed, thereby promoting penetration of the stabilizer(s) and/or sensitizer(s) into the tissue or providing a greater surface area of tissue to be in contact with the stabilizer(s) and/or sensitizer(s). The compounds that cause macromolecules in the tissue to become less compact, or relaxed, may also be applied prior to introduction of the stabilizer(s) and/or sensitizer(s), which may then be introduced in a similar solution followed by application of a solution containing a similar amount of stabilizer(s) and/or sensitizer(s) but a reduced amount of the compounds that cause macromolecules in the tissue to become less compact, or relaxed. Repeated applications of such solutions, with progressively lower amounts of compounds that cause macromolecules in the tissue to become less compact, or relaxed, may subsequently be applied.

The compounds that promote penetration may be used alone or in combination, such as a combination of a compound that causes macromolecules in the tissue to become less compact and a compound that causes an increase in the distance between molecules in the tissue.

Further, in those embodiments of the present invention wherein the stabilizer(s) and/or sensitizer(s) is cationic, one or more anionic compounds may be added to the solution containing the stabilizer(s) and/or sensitizer(s) prior to and/or during application thereof to the tissue. The anionic compound(s) may also be applied prior to introduction of the stabilizer(s) and/or sensitizer(s), which may then be introduced in a similar solution followed by application of a solution containing a similar amount of stabilizer(s) and/or sensitizer(s) but a reduced amount of the anionic compound(s). Repeated applications of such solutions, with progressively lower amounts of anionic compound(s) may subsequently be applied.

Similarly, in those embodiments of the present invention wherein the stabilizer(s) and/or sensitizer(s) is anionic, one or more cationic compounds may be added to the solution containing the stabilizer(s) and/or sensitizer(s) prior to and/or during application thereof to the tissue. The cationic compound(s) may also be applied prior to introduction of the stabilizer(s) and/or sensitizer(s), which may then be introduced in a similar solution followed by application of a solution containing a similar amount of stabilizer(s) and/or sensitizer(s) but a reduced amount of the cationic compound(s). Repeated applications of such solutions, with progressively lower amounts of cationic compound(s) may subsequently be applied.

It will be appreciated that the combination of one or more of the features described herein may be employed to further minimize undesirable effects upon the one or more tissues caused by irradiation, while maintaining adequate effectiveness of the irradiation process on the biological contaminant(s) or pathogen(s). For example, in addition to the use of a stabilizer, a particular tissue may also be lyophilized, held at a reduced temperature and kept under vacuum prior to irradiation to further minimize undesirable effects.

The sensitivity of a particular biological contaminant or pathogen to radiation is commonly calculated by determining the dose necessary to inactivate or kill all but 37% of the agent in a sample, which is known as the D₃₇ value. The desirable components of a tissue may also be considered to have a D₃₇ value equal to the dose of radiation required to eliminate all but 37% of their desirable biological and physiological characteristics.

In accordance with certain preferred methods of the present invention, the sterilization of one or more tissues is conducted under conditions that result in a decrease in the D₃₇ value of the biological contaminant or pathogen without a concomitant decrease in the D₃₇ value of the one or more tissues. In accordance with other preferred methods of the present invention, the sterilization of one or more tissues is conducted under conditions that result in an increase in the D₃₇ value of the tissue material. In accordance with the most preferred methods of the present invention, the sterilization of one or more tissues is conducted under conditions that result in a decrease in the D₃₇ value of the biological contaminant or pathogen and a concomitant increase in the D₃₇ value of the one or more tissues.

In accordance with certain preferred methods of the present invention, the sterilization of one or more tissues is conducted under conditions that reduce the possibility of the production of neo-antigens. In accordance with other preferred embodiments of the present invention, the sterilization of one or more tissues is conducted under conditions that result in the production of substantially no neo-antigens. The present invention also includes tissues sterilized according to such methods.

In accordance with certain preferred methods of the present invention, the sterilization of one or more tissues is conducted under conditions that reduce the total antigenicity of the tissue(s). In accordance with other preferred embodiments of the present invention the sterilization of one or more tissues is conducted under conditions that reduce the number of reactive allo-antigens and/or xeno-antigens in the tissue(s). The present invention also includes tissues sterilized according to such methods.

A particularly preferred tissue for use with the methods of the present invention is collagen. According to certain embodiments of the present invention, collagen is employed as a model tissue for determining optimal conditions, such as preferred rates of irradiation, temperatures, residual solvent content, and the like, for sterilizing a given tissue type with gamma radiation without rendering the tissue unsafe and/or ineffective for its intended purpose. Thus, another preferred embodiment of the present invention is directed to an assay for determining the optimal conditions for sterilizing a tissue that contains collagen without adversely affective a predetermined biological characteristic or property thereof, which comprises the steps of: (i) irradiating collagen under a pre-determined set of conditions effective to sterilize the tissue; (ii) determining the turbidity of the irradiated collagen; and (iii) repeating steps (i) and (ii) with a different pre-determined set of conditions until the turbidity of the irradiated collagen reaches a pre-determined acceptable level.

According to certain preferred embodiments of the present invention, one or more tissues sterilized according to the methods described herein may be introduced into a mammal in need thereof for prophylaxis or treatment of a condition or disease or malfunction of a tissue. Methods of introducing such tissue into a mammal are known to those skilled in the art.

When employed in such embodiments, one or more tissues sterilized according to the methods described herein do not produce sufficient negative characteristics in the tissue(s) following introduction into the mammal to render the tissue(s) unsafe and/or ineffective for the intended use thereof. Illustrative examples of such negative characteristics include, but are not limited to, inflammation and calcification. Such negative characteristics may be detected by any means known to those skilled in the art, such as MRIs, CAT scans and the like.

According to particularly preferred embodiments of the present invention, sterilization of the one or more tissues is conducted after the tissue(s) is packaged, i.e. as a terminal sterilization process.

### EXAMPLES

The following examples are illustrative, but not limiting, of the present invention. Other suitable modifications and adaptations are of the variety normally encountered by those skilled in the art and are fully within the spirit and scope of the present invention. For example, heart valves from animal species other than pig, such as bovine or human, are encompassed by this technology, as are heart valves from transgenic mammals. In addition, heart valves prepared/modified by practice of the present invention may be used for transplantation into any animal, particularly into mammals. Furthermore, the principles of the technology of the present invention may be practiced on animal tissues and organs other than heart valves. Unless otherwise noted, all irradiation was accomplished using a ⁶⁰Co source.

### Example 1

In this experiment, porcine heart valves were gamma irradiated in the presence of polypropylene glycol 400 (PPG400) and, optionally, a scavenger, to a total dose of 30 kGy (1.584 kGy/hr at −20° C.).

Materials:

Tissue—Porcine Pulmonary Valve (PV) Heart valves were harvested prior to use and stored.

Tissue Preparation Reagents—

Polypropylene Glycol 400. Fluka: cat#81350, lot#386716/1
Trolox C. Aldrich: cat#23,881-3, lot#02507TS
Coumaric Acid. Sigma: cat#C-9008, lot#49H3600
n-Propyl Gallate. Sigma: cat#P-3130, lot#117H0526
α-Lipoic Acid. CalBiochem: cat#437692, lot#B34484
Dulbecco's PBS. Gibco BRL: cat#14190-144, lot#1095027
2.0 ml Screw Cap tubes. VWR Scientific Products: cat#20170-221, lot#0359
   Tissue Hydrolysis Reagents—
Nerl H₂O. NERL Diagnostics: cat#9800-5, lot#03055151
Acetone. EM Science: cat#AX0125-5, lot#37059711
6 N constant boiling HCl. Pierce: cat#24309, lot#BA42184
Int-Pyd (Acetylated Pyridinoline) HPLC Internal Standard. Metra Biosystems Inc.: cat#8006,
   lot#9H142, expiration February 2002, Store at ≦−20° C.
Hydrochloric Acid. VWR Scientific: cat#VW3110-3, lot#n/a
Heptafluorobutyric Acid (HFBA) Sigma: cat#H-7133, lot#20K3482
   FW 214.0 store at 2-8° C.
SP-Sephadex C-25 resin. Pharmacia: cat#17-0230-01, lot#247249 (was charged with NaCl as per manufacturer suggestion)
   Hydrolysis vials—10 mm×100 mm vacuum hydrolysis tubes. Pierce: cat#29560, lot #BB627281
   Heating module—Pierce, Reacti-therm.: Model #18870, S/N 1125000320176
   Savant—Savant Speed Vac System:
Speed Vac Model SC110, model #SC110-120, serial #SC110-SD171002-1H
   a. Refrigerated Vapor Trap Model RVT100, model #RVT100-120V, serial #RVT100-58010538-1B
   b. Vacuum pump, VP 100 Two Stage Pump Model VP100, serial #93024
      Column—Phenomenex, Luna 5μ C18(2) 100 Å, 4.6×250 mm. Part #00G-4252-E0, S/N #68740-25, B/N#5291-29
      HPLC System:
Shimadzu System Controller SCL-10A
Shimadzu Automatic Sample Injector SIL-10A (50 μl loop)
Shimadzu Spectrofluorometric Detector RF-10A
   Shimadzu Pumps LC-10AD
Software—Class-VP version 4.1
   Low-binding tubes—MiniSorp 100×15 Nunc-Immunotube. Batch #042950, cat#468608
   Methods:
   A. Preparation of Stabilizer Solutions:
   Trolox C:

The 0.5 M solution was not soluble; therefore additional PPG was added. After water bath sonication at 25° C. and above for at least 30 minutes, Trolox C is soluble at 125 mM.

Coumaric Acid:

Water bath sonicated at 25° C. and above for approximately 15 minutes—not 100% soluble. An additional 1 ml PPG was added and further water bath sonicated.

n-Propyl Gallate:

The 0.5M solution was soluble after a 20-30 minute water bath sonication.

1 M α-Lipoic Acid:

Very soluble after 10 minute water bath sonication.

### Final Stocks of Scavengers

125 mM Trolox C—4 ml
0.5 M Coumaric acid—2 ml
0.5 M n-Propyl Gallate—2 ml
1 M Lipoic Acid—2 ml
   B. Treatment of Valves Prior to Gamma-Irradiation.

1. PV heart valves were thawed on wet ice.

2. Cusps were dissected out from each valve and pooled into 50 ml conical tubes containing cold Dulbecco's PBS.

3. Cusps were washed in PBS at 4° C. for approximately 1.5 hrs; changing PBS during that time a total of 6 times.

4. 2 cusps were placed in each of six 2 ml screw cap tube.

5. 1.2 ml of PPG were added to two tubes (one of these tubes was designated 0 kGy and the other tube was designated 30 kGy):
1.2 ml of 125 mM Trolox C in PPG were added to another two tubes
1.2 ml of SCb stabilizer mixture—comprising of 1.5 ml 125 mM Trolox C, 300 μl 1 M Lipoic Acid, 600 μl 10.5 M Coumaric Acid and 600 μl 0.5 M n-Propyl Gallate (Final concentrations: 62.5 mM, 100 mM, 100 mM and 100 mM respectively) were added to the final two tubes.

6. Tubes were incubated at 4° C., with rocking for about 60 hours.

7. Stabilizer solutions and cusps were transferred into 2 ml glass vials for gamma-irradiation.

8. All vials were frozen on dry ice.

9. Control samples were kept in-house at −20° C.

C. Gamma-Irradiation of Tissue.

Samples were irradiated at a rate of 1.584 kGy/hr at −20° C. to a total dose of 30 kGy.

D. Processing Tissue for Hydrolysis/Extraction.

1. Since PPG is viscous, PBS was added to allow for easier transfer of material.

2. Each pair of cusps (2 per condition) were placed into a 50 ml Falcon tube filled with cold PBS and incubated on ice—inverting tubes periodically.

3. After one hour PBS was decanted from the tubes containing cusps in PPG/0 kGy and PPG/30 kGy and replenished with fresh cold PBS. For the PPG samples containing Trolox C or SCb stabilizer mixture, fresh 50 ml Falcon tubes filled with cold PBS were set-up and the cusps transferred.

4. An additional 3 washes were done.

5. One cusp was transferred into a 2 ml Eppendorf tube filled with cold PBS for extraction. The other cusp was set-up for hydrolysis.

E. Hydrolysis of Tissue.

1. Each cusp was washed 6× with acetone in an Eppendorf tube (approximately 1.5 ml/wash).

2. Each cusp was subjected to SpeedVac (with no heat) for approximately 15 minutes or until dry.

3. Samples were weighed, transferred to hydrolysis vials and 6 N HCl added at a volume of 20 mg tissue/ml HCl:

| Sample ID | Dry Weight (mg) | μl 6 N HCl |
|---|---|---|
| 1. PPG/0 | 6.49 | 325 |
| 2. PPG/30 | 7.26 | 363 |
| 3. PPG T/0 | 5.80 | 290 |
| 4. PPG T/30 | 8.20 | 410 |
| 5. PPG SCb/0 | 6.41 | 321 |
| 6. PPG SCb/30 | 8.60 | 430 |

4. Samples were hydrolyzed at 110° C. for approximately 23 hours.

5. Hydrolysates were transferred into Eppendorf tubes and centrifuged @12,000 rpm for 5 min.

6. Supernatent was then transferred into a clean Eppendorf.

7. 50 μl of hydrolysate was diluted in 8 ml Nerl H₂O (diluting HCl to approximately 38 mM).

8. Spiked in 200 μl of 2×int-pyd. Mixed by inversion. (For 1600 μl 2×int-pyd:160 μl 20×int-pyd+1440 μl Nerl H₂O.)

9. Samples were loaded onto SP-Sephadex C25 column (approximately 1×1 cm packed bed volume) that had been equilibrated in water. (Column was pre-charged with NaCl)

10. Loaded flow through once again over column.

11. Washed with 20 ml 150 mM HCl.

12. Eluted crosslinks with 5 ml 2 N HCl into a low binding tube.

13. Dried entire sample in Savant.

F. Analysis of Hydrolysates.

Set-up the following:

| Sample | μl | μl H₂O | μl HFBA |
|---|---|---|---|
| 1. PPG/10 kGy | 18 | 180 | 2 |
| 2. PPG/30 kGy | 59 | 139 | 2 |
| 3. PPG T/0 kGy | 67 | 171 | 2 |
| 4. PPG T/30 kGy | 64 | 134 | 2 |
| 5. PPG SCb/0 kGy | 10 | 188 | 2 |
| 6. PPG SCb/30 kGy | 32 | 166 | 2 |

Results:

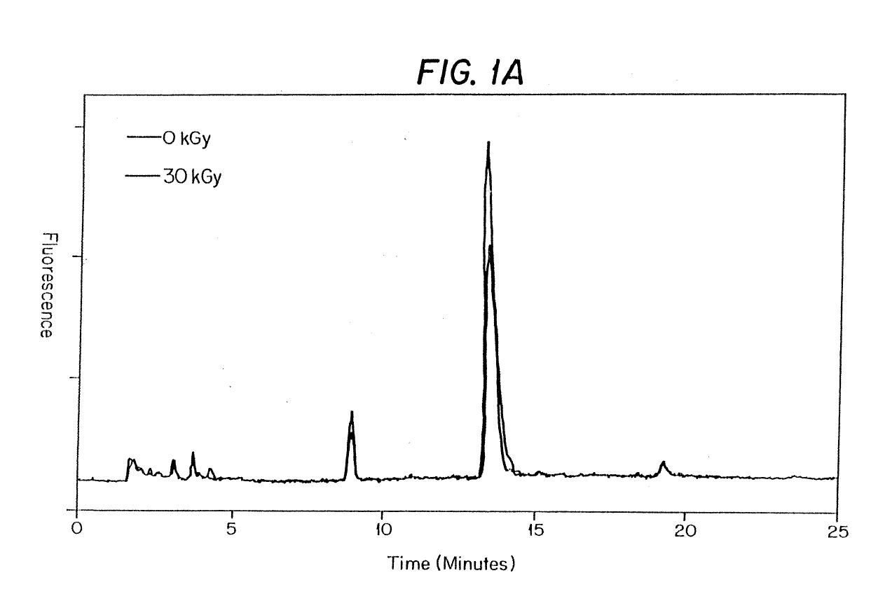

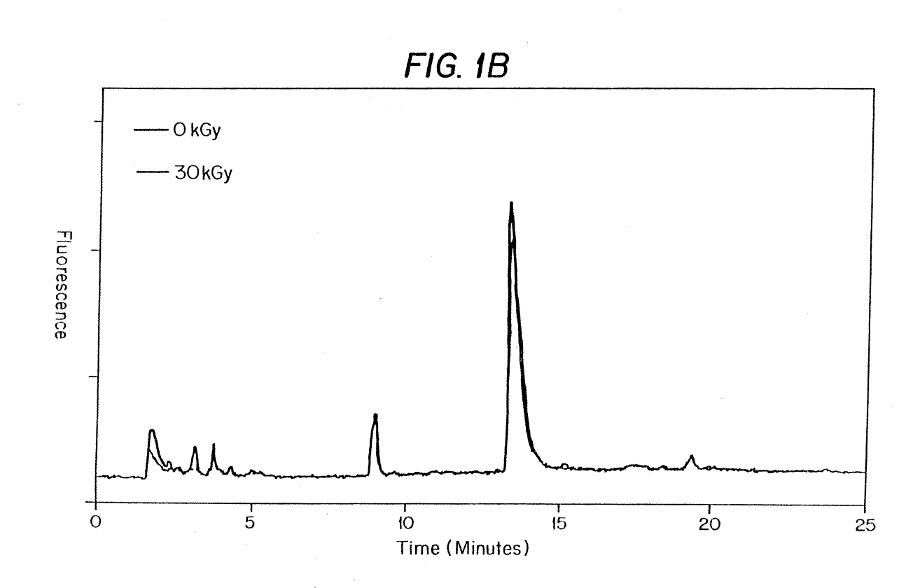

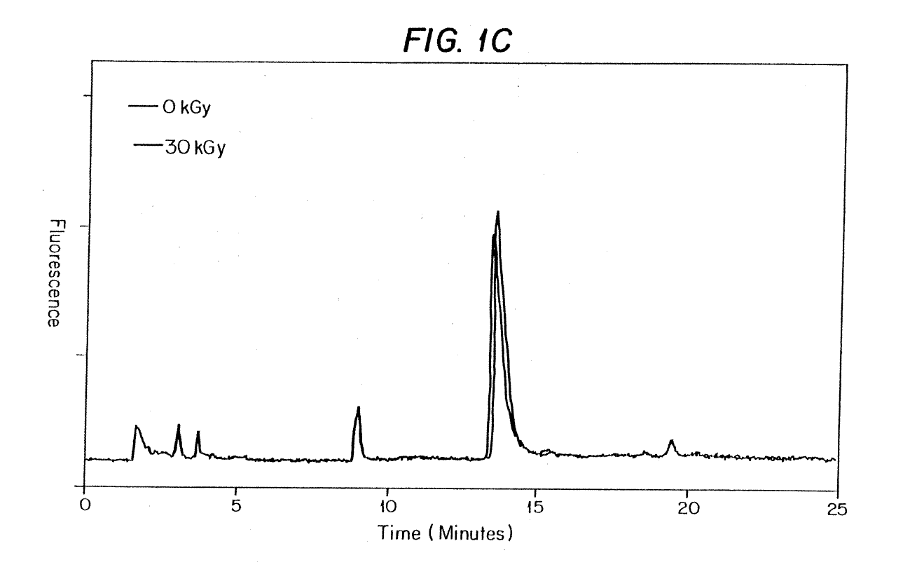

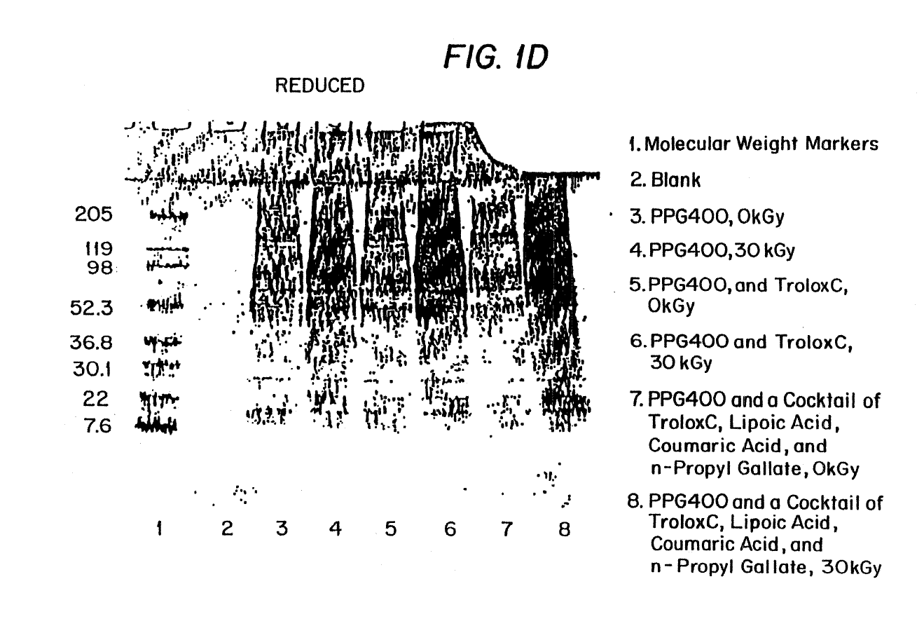

The HPLC results are shown in FIGS. 1A-1C. In the presence of PPG 400, the results were nearly identical whether the heart valve had been irradiated or not. The addition of a single stabilizer (trolox C) or a stabilizer mixture produced even more effective results. The gel analysis, shown in FIG. 1D, confirmed the effectiveness of the protection provided by these conditions.

### Example 2

In this experiment, the effects of gamma irradiation were determined on porcine heart valve cusps in the presence of 50% DMSO and, optionally, a stabilizer, and in the presence of polypropylene glycol 400 (PPG400).

Preparation of Tissue for Irradiation:

1. 5 vials of PV and 3 vials of atrial valves (AV) were thawed on ice.

2. Thaw media was removed and valves rinsed in beaker filled with PBS.

3. Transferred each valve to 50 ml conical containing PBS. Washed by inversion and removed.

4. Repeated wash 3 times.

5. Dissected out the 3 cusps (valves).

6. Stored in PBS in 2 ml screw top Eppendorf Vials (Eppendorfs) and kept on ice.

Preparation of Stabilizers:

All stabilizers were prepared so that the final concentration of DMSO was 50%.

1 M Ascorbate in 50% DMSO:

Aldrich: cat#26,855-0, lot#10801HU

200 mg dissolved in 300 μl H₂O. Add 500 μl DMSO. The volume was adjusted to 1 ml with H₂O. Final pH was ≈8.0.

1 M Coumaric Acid:

Sigma: cat#C-9008, lot#49H3600. MW 164.2

Dissolve 34.7 mg in 106 μl DMSO, pH≈3.0

138 μl H₂O was added. Sample precipitated out of solution.

Coumaric went back into solution once pH was adjusted to 7.5 with 1 N NaOH.

1 M n-Propyl Gallate:

Sigma: cat#P-3130, lot#117H0526. MW 212.2

Dissolve 58.2 mg in 138 μl DMSO.

Add 138 μl H₂O. Final pH is 6.5 or slightly lower.

Stabilizer Mixture (SM-a):

1.0 ml 500 mM Ascorbate

500 μl 1 M Coumaric Acid

300 μl 1 M n-propyl gallate

1.2 ml 50% DMSO

3.0 ml

Method:

1.6 ml of a solution (stabilizer mixture or PPG400) was added to each sample and then the sample was incubated at 4° C. for 2.5 days. Valves and 1 ml of the solution in which they were incubated were then transferred into 2 ml irradiation vials. Each sample was irradiated with gamma irradiation at a rate of 1.723 kGy/hr at 3.6° C. to a total dose of 25 kGy.

Hydrolysis of Tissue:

1. Washed each cusp 6 times with acetone in a 2 ml Eppendorf Vial.

2. After final acetone wash, dried sample in Savant (without heat) for approximately 10-15 minutes or until dry.

3. Weighed the samples, transferred them to hydrolysis vials and then added 6 N HCl at a volume of 20 mg tissue/ml HCl:

| Sample ID | Dry Weight (mg) | μl 6 N HCl |
|---|---|---|
| 1. PBS/0 kGy | 11.4 | 570 |
| 2. PBS/25 kGy | 6.0 | 300 |
| 3. DMSO/0 kGy | 6.42 | 321 |
| 4. DMSO/25 kGy | 8.14 | 407 |
| 5. DMSO/SM-a/0 kGy | 8.7 | 435 |
| 6. DMSO/SM-a/25 kGy | 8.15 | 408 |
| 7. PPG/0 kGy | 13.09 | 655 |
| 8. PPG/25 kGy | 10.88 | 544 |

SM = Stabilizer Mixture as defined above.

5. Samples were hydrolyzed at 110° C. for approximately 23 hours.

6. Hydrolysates were transferred into Eppendorf vials and centrifuged at 12,000 rpm for 5 min.

7. Supernatent was transferred into a clean Eppendorf vial.

8. 50 μl hydrolysate was diluted in 8 ml Nerl H₂O (diluting HCl to approximately 37 mM).

9. Spiked in 200 μl of 2×int-pyd. Mixed by inversion. (For 2000 μl 2×int-pyd: 200 μl 20×int-pyd+1.8 ml Nerl H₂O.)

10. Samples were loaded onto SP-Sephadex C25 column (approximately 1×1 cm packed bed volume) that had been equilibrated in water. (Column was pre-charged with NaCl)

11. Loaded flow through once again over column.

12. Washed with 20 ml 150 mM HCl.

13. Eluted crosslinks with 5 ml 2 N HCl into a low binding tube. 50 ml 2 N HCl:8.6 ml concentrated HCl adjusted to a volume of 50 ml with Nerl H₂O.

14. Dried entire sample in Savant.

Guanidine HCl Extraction and DEAE-Sepharose Purification of Proteoglycans:

4M Guanidine HCl Extraction:

1. Removed all three cusps from gamma irradiation vial and transferred to separate 50 ml conical tube.

2. Washed cusps five times with 50 ml dPBS (at 4° C. over approx. 5 hours) and determined wet weight of one cusp after drying on Kimwipe.

3. Transferred one cusp from each group to 1.5 ml microfuge tube and added appropriate volume of 4M guanidine HCl/150 mM sodium acetate buffer pH 5.8 with 2 μg/ml protease inhibitors (aprotinin, leupeptin, pepstatin A) to have volume to tissue ratio of 15 (see Methods in Enzymology Vol. 144 p. 321—for optimal yield use ratio of 15 to 20).

4. Diced cusps into small pieces with scissors.

5. Nutated at 4° C. for ˜48 hours.

6. Centrifuged at 16,500 RPM on Hermle Z-252M, at 4° C. for 10 min.

7. Collected guanidine soluble fraction and dialyzed against PBS in 10K MWCO Slide-A-Lyzer overnight against 5 L PBS (3 slide-a-lyzers with one 5 L and 5 slide-a-lyzers in another 5 L) to remove guanidine.

8. Changed PBS and dialyzed for additional 9 hours at 4° C. with stirring.

9. Collected the dialysate and stored at 4° C.

10. Centrifuged at 16,500 RPM on Hermle Z-252M, at 4° C. for 5 min

11. Removed PBS soluble fraction for DEAE-Sepharose chromatography.

DEAE-Sepharose Chromatography

1. Increased the NaCl concentration of 500 μl of PBS soluble guanidine extract to 300 mM NaCl (Assumed PBS soluble fractions were already at ˜150 mM NaCl, so added 15 μl 5M NaCl stock to each 500 μl sample).

2. Equilibrated ˜1 ml of packed DEAE-Sepharose (previously washed with 1M NaCl/PB pH 7.2) into 300 mM NaCl/PB pH 7.2 (Note: To make 300 mM NaCl/PB pH7.2—added 3 ml of 5M NaCl stock to 100 ml PBS).

3. Added 200 μl of 1:1 slurry of resin to 515 μL of GuHCl extracts (both at 300 mM NaCl).

4. Nutated at ambient temperature for ˜one hour.

5. Centrifuged gently to pellet resin.

6. Removed “unbound” sample and stored at −20° C.

7. Washed resin 5 times with ˜1.5 ml of 300 mM NaCl/PBS pH7.2.

8. After last wash, removed all extra buffer using a 100 μl Hamilton syringe.

9. Eluted at ambient temperature with three 100 μl volumes of 1M NaCl/PB pH 7.2 and stored at −20° C.

SDS-PAGE:

5-20% gradient gels for analysis of PBS soluble Guanidine HCl extracts and DEAE-Sepharose chromatography.

1. Gel#1: GuHCl extracts/PBS soluble fractions—Toluidine blue and then Coomassie blue stained.

2. Gel#2: DEAE-Sepharose Eluant Fraction#1—Toluidine Blue stained then Coomassie Blue stained.

Quantification of Collagen Crosslinks by HPLC:

1. Prepared 100-200 μl 1× solution in 1% heptafluorobutyric acid (HFBA).
2. Injected 50 μl on C18 HPLC column equilibrated with mobile phase.
3. Spectrofluorometer was set for excitation at 295 nm and emission at 395 nm.
4. Calculated the integrated fluorescence of Internal-Pyridinoline (Int-Pyd) per 1 μl of 1× solution of Int-Pyd.
   Results:

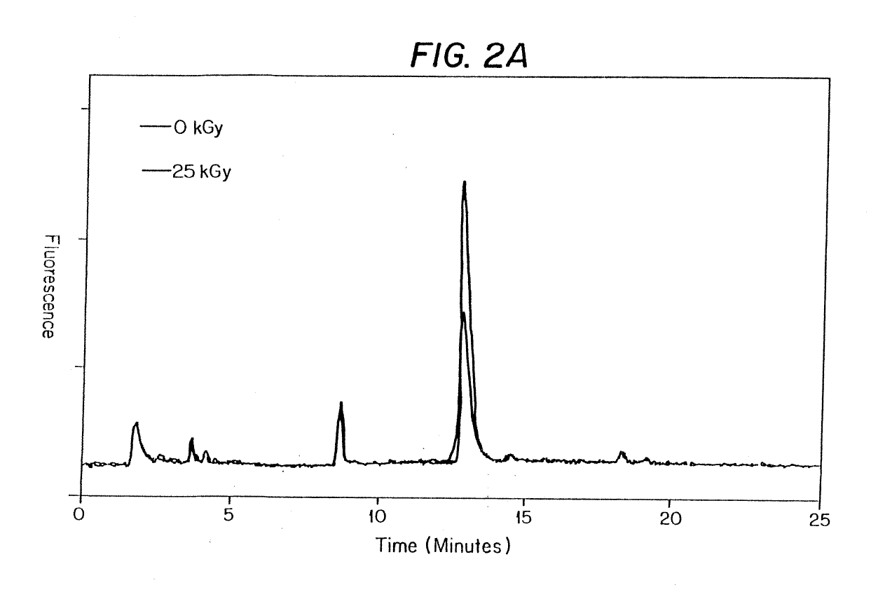

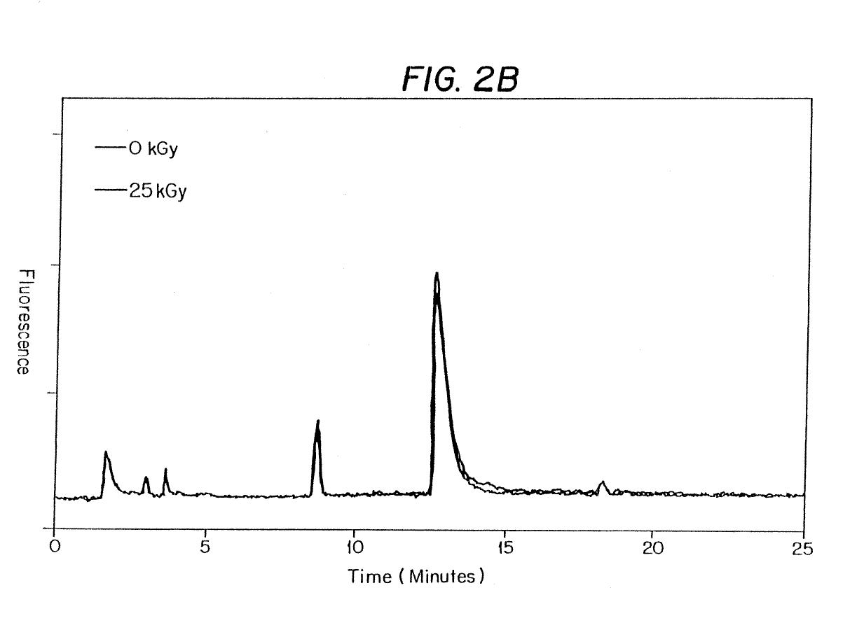

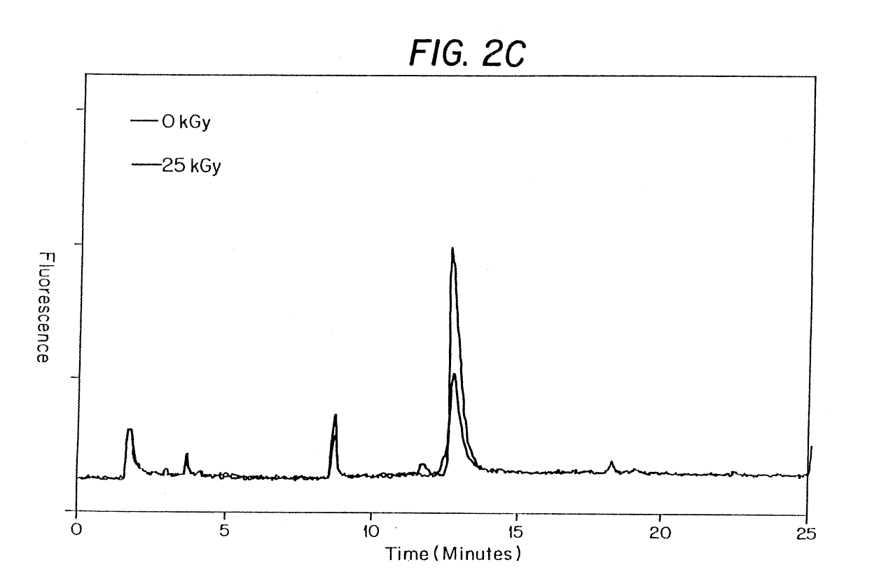

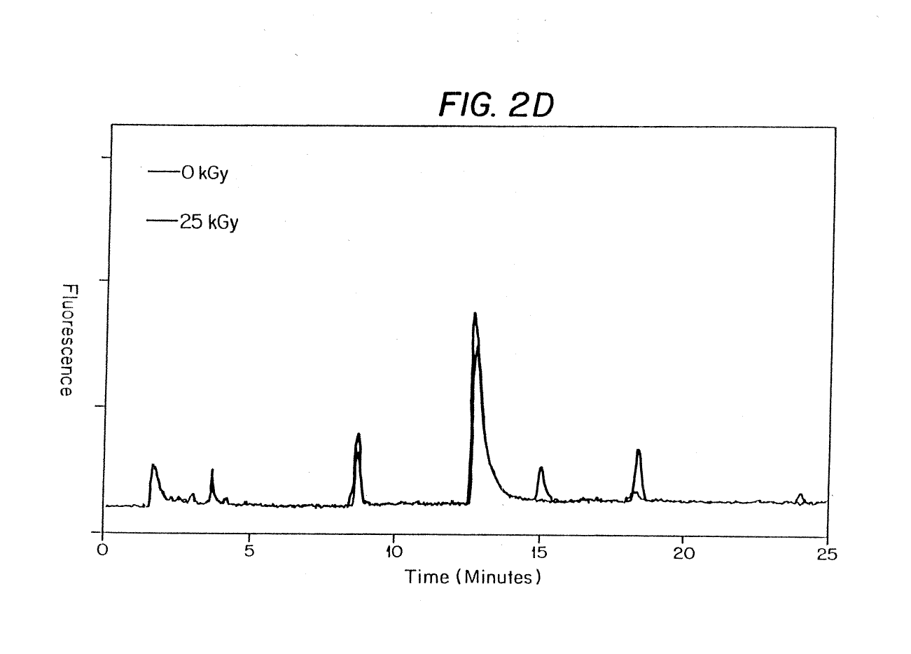

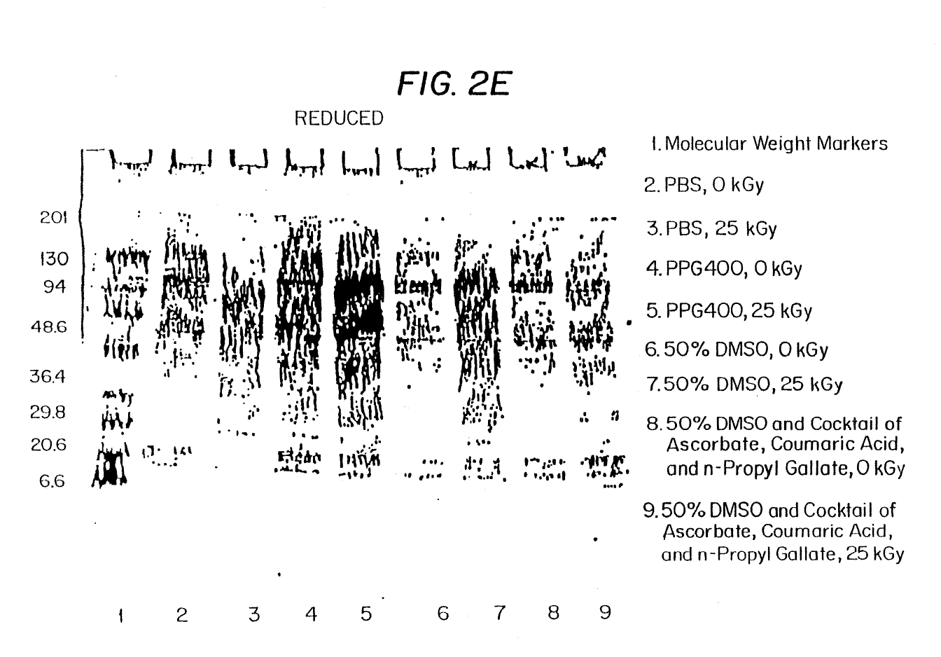

The HPLC results are shown in FIGS. 2A-D. The major peak represents the Internal-Pyridinoline (int-Pyd) peak. Irradiation in an aqueous environment (PBS) produced pronounced decreases in the smaller peaks (FIG. 2A). Reduction of the water content by the addition of a non-aqueous solvent (PPG 400) produced a nearly superimposable curve (FIG. 2B). DMSO was less effective (FIG. 2C), while DMSO plus a mixture of stabilizers (FIG. 2D) was more effective at preserving the major peak although some minor peaks increased somewhat. The area under the pyd peak for each sample was calculated as shown in the table below. These results confirm the above conclusions and show that the amino acid crosslinks (pyd) found in mature collagen are effectively conserved in the samples containing PPG and DMSO with a scavenger mixture. Gel analysis is shown in FIG. 2E and reflects the major conclusions from the HPLC analysis, with significant loss of bands seen in PBS and retention of the major bands in the presence of non-aqueous solvents.

| Sample | Area of Pyd Peak |
|---|---|
| PBS/0 kGy | 94346 |
| PBS/25 kGy | 60324 |
| DMSO/0 kGy | 87880 |
| DMSO/25 kGy | 49030 |
| DMSO/SM/0 kGy | 75515 |
| DMSO/SM/25 kGy | 88714 |
| PPG/0 kGy | 99002 |
| PPG/25 kGy | 110182 |

### Example 3

In this experiment, frozen porcine AV heart valves soaked in various solvents were gamma irradiated to a total dose of 30 kGy at 1.584 kGy/hr at −20° C.

Materials:

1. Porcine heart valve cusps were obtained and stored at −80° C. in a cryopreservative solution (Containing Fetal calf serum, Penicillin-Streptomycin, M199 media, and approximately 20% DMSO).
2. Dulbecco's Phosphate Buffered Saline. Gibco BRL: cat#14190-144, lot#1095027
3. 2 ml screw cap vials. VWR: cat#20170-221, lot #0359
4. 2 ml glass vials. Wheaton: cat#223583, lot#370000-01
5. 13 mm stoppers. Stelmi: 6720GC, lot#G006/5511
6. DMSO. JT Baker: cat#9224-01, lot#H40630
7. Sodium ascorbate. Aldrich: cat#26,855-0, lot 10801HU; prepared as a 2M stock in Nerl water.
8. Fetal calf serum
9. Penicillin-Streptomycin
10. M199 media
11. DMSO
   Methods:
   Cryopreservative Procedure:

Preparation of Solutions

Freeze Medium:
Fetal calf serum (FCS) (10%)=50 ml
Penicillin-Streptomycin=2.5 ml
M199=QS 500 ml

2M DMSO
DMSO=15.62 g
Freeze Medium=QS100 ml

3M DMSO
DMSO=23.44 g
Freeze Medium=QS100 ml

Preparation of Tissue
1. Placed dissected heart valves (with a small amount of conduit/muscle attached) into glass freezing tubes (label with pencil).
2. Added 2 ml of freeze medium.
3. At 21° C., added 1 ml 2M DMSO solution.
4. At 5 minutes, added 1 ml 2M DMSO solution.
5. At 30 minutes, added 4 ml 3M DMSO solution.
6. At 45 minutes and 4° C., placed freezing tubes on ice.
7. At 50 minutes and −7.2° C., seeded bath, which is an alcohol filled tank inside the cryopreservation machine and is used to lower the temperature quickly.
8. At 55 minutes and −7.2° C., nucleated. Nucleation is a processing step that allows the tissue to freeze evenly and quickly without much ice formation. This is done by placing a steel probe in a liquid nitrogen canister, touching the probe to the outside of the freezing tube at the surface of the solution, waiting for ice formation, shaking the tube and placing the tube in the bath.
9. At 70 minutes, cooled to −40° C. at 1° C./minute. Removed from bath and placed in canister of liquid N₂, and stored in cryogenic storage vessel.
   Procedure for Irradiation of Heart Valves:

1. Thawed AV heart valve cusps on wet ice.

2. Pooled cusps into 50 ml tubes.

3. Washed cusps with ˜50 ml dPBS at 4° C. while nutating. Changed PBS 5 times over the course of 5 hrs.

4. Transferred cusps into 2 ml screw cap tubes (2 cusps/tube).

5. Added 1.0 ml of the following to two of each of two tubes: dPBS, 50% DMSO and 50% DMSO with 200 mM sodium ascorbate (2M sodium ascorbate stock was diluted as follows: 400% (2M)+1.6 ml water+2 ml 100% DMSO).

6. Incubated tubes at 4° C. with nutating for ˜46 hours.

7. Transferred solutions and cusps to glass 2 ml vials, stoppered and capped.

8. All vials were frozen on dry ice.

9. Frozen samples were then irradiated at −20° C. at a rate of 1.584 kGy/hr to a total dose of 30 kGy.

Results:

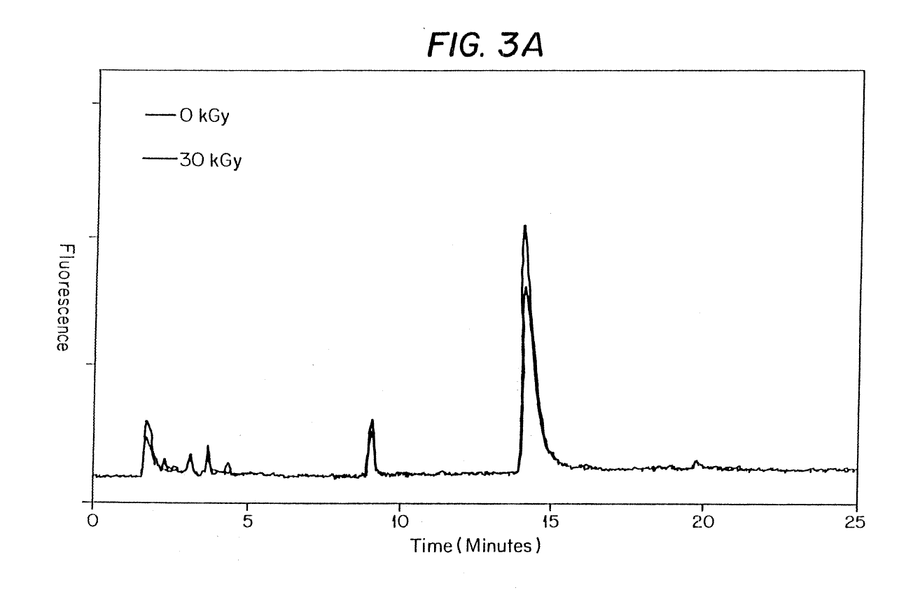

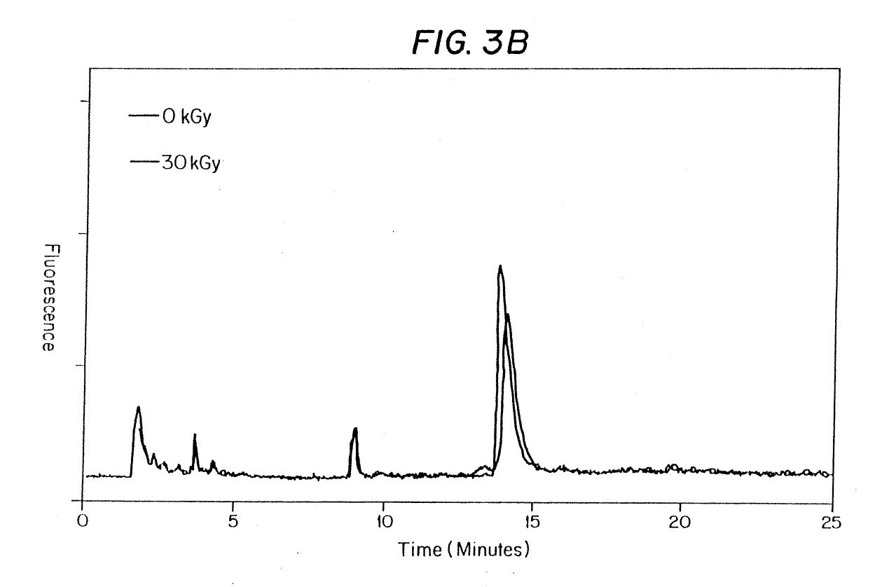

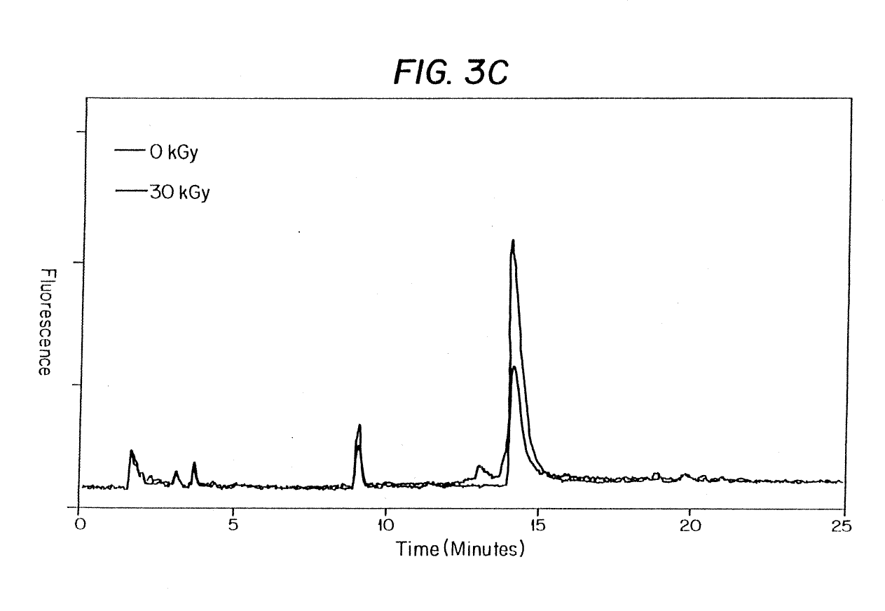

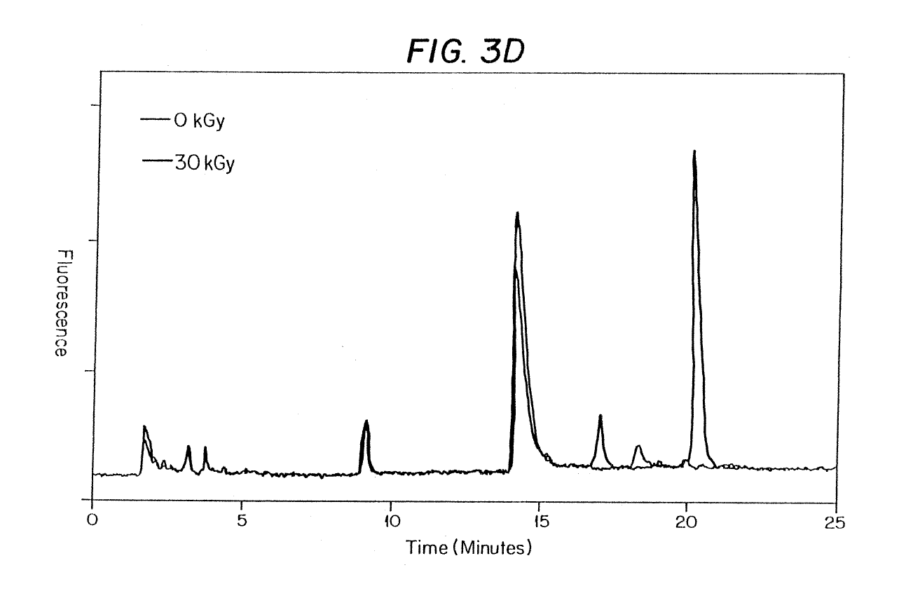

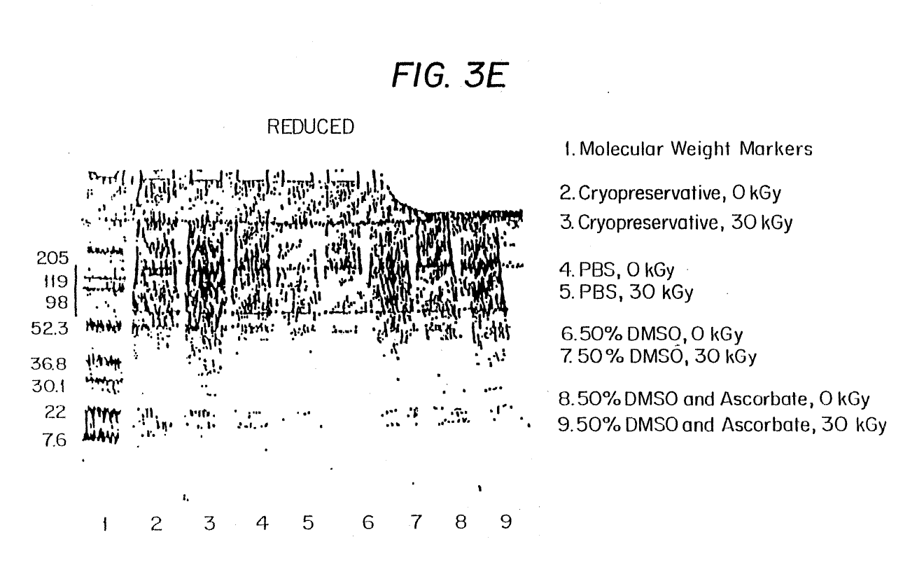

The results of the HPLC analysis are shown in FIGS. 3A-3D. Irradiation in an aqueous environment (PBS) produced decreases in the smaller peaks (FIG. 3A). Reduction of the water content by the addition of a non-aqueous solvent (20% DMSO) reproduced these peaks more faithfully (FIG. 3B). Increasing the DMSO concentration to 50% was slightly more effective (FIG. 3C), while DMSO plus a mixture of stabilizers (FIG. 3D) was very effective at preserving both the major and minor peaks (the additional new peaks are due to the stabilizers themselves). Gel analysis is shown in FIG. 3E and reflects the major conclusions from the HPLC analysis, with significant loss of bands seen in PBS and retention of the major bands in the presence of non-aqueous solvents with and without stabilizers.

### Example 4

In this experiment, frozen porcine AV heart valves soaked in various solvents were gamma irradiated to a total dose of 45 kGy at approximately 6 kGy/hr at −70° C.

Materials:

1. Porcine heart valve cusps were obtained and stored at −80° C. in a cryopreservative solution (Same solution as that in Example 3).
2. Dulbecco's Phosphate Buffered Saline (dPBS). Gibco BRL: cat#14190-144, lot 1095027
3. 2 ml screw cap vials. VWR: cat#20170221, lot #0359
4. 2 ml glass vials. Wheaton: cat#223583, lot#370000-01
5. 13 mm stoppers. Stelmi: 6720GC, lot#G006/5511
6. DMSO. JT Baker: cat#9224-01, lot#H40630
7. Sodium ascorbate. Aldrich: cat#26,855-0, lot 10801HU; prepared as a 2M stock in Nerl water.
8. Polypropylene glycol 400 (PPG400). Fluka: cat#81350, lot#386716/1
   Methods:

Cryopreservative Procedure is the same as that shown in Example 3.

1. Thawed AV heart valve cusps on wet ice. Dissected out cusps and washed the pooled cusps 6 times with cold PBS.

2. Dried each cusp and transferred cusps into 2 ml screw cap tubes (2 cusps/tube).

3. Added 1.2 ml of the following to two of each of two tubes: dPBS, dPBS with 200 mM sodium ascorbate, PPG400, PPG400 for rehydration, 50% DMSO and 50% DMSO with 200 mM sodium ascorbate (2M sodium ascorbate stock was diluted as follows: 400 μL (2M)+1.6 ml water+2 ml 100% DMSO).

4. Incubated tubes at 4° C. with nutating for ˜46 hours.

5. Replaced all solutions with fresh solutions (with the following exception: for one PPG400 set, PPG400 was removed, the cusp washed with PBS+200 mM ascorbate, which was then removed and replaced with fresh PBS+200 mM ascorbate).

6. Incubated tubes at 4° C. with nutating for ˜46 hours.

7. Changed the solution on the PPG400 dehyd./PBS+ascorbate rehydration cusps prepared in step 5.

8. Incubated tubes at 4° C. with nutating for 6 hours.

9. Transferred solutions and cusps to glass 2 ml vials, stoppered and capped.

10. All vials were frozen on dry ice.

11. 5 Frozen samples were then irradiated at −70° C. at a rate of 6 kGy/hr to a total dose of 45 kGy.

Results:

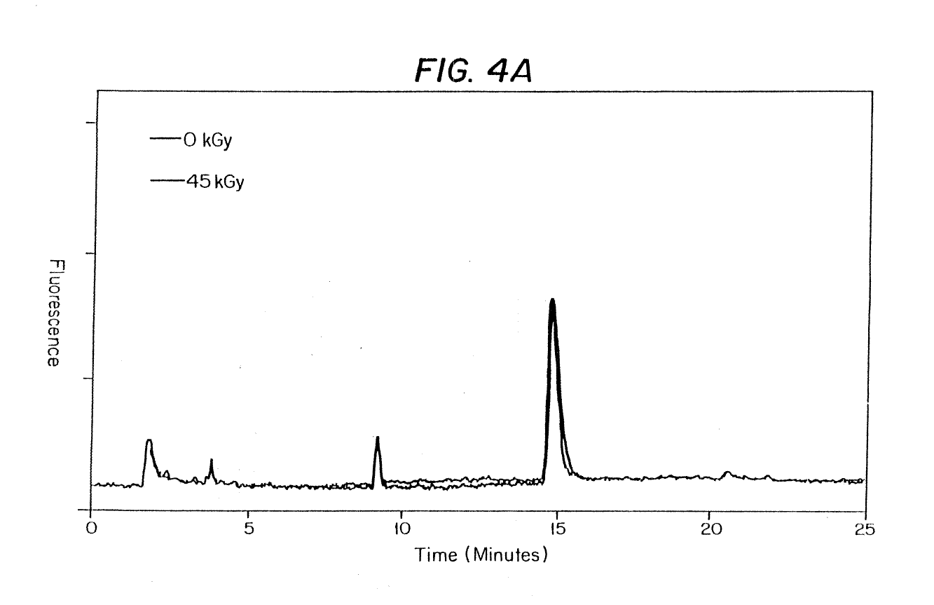

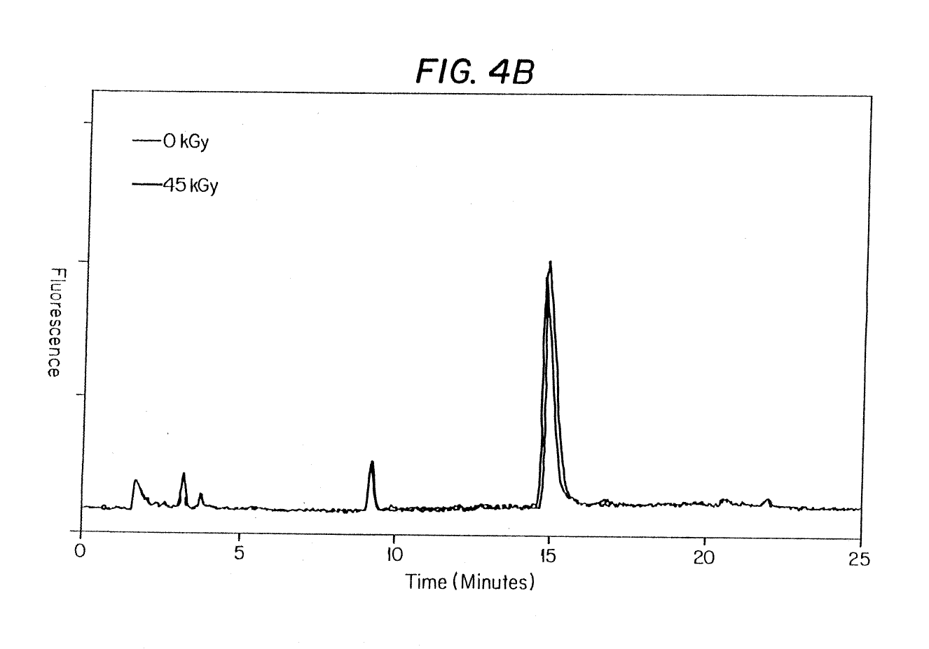

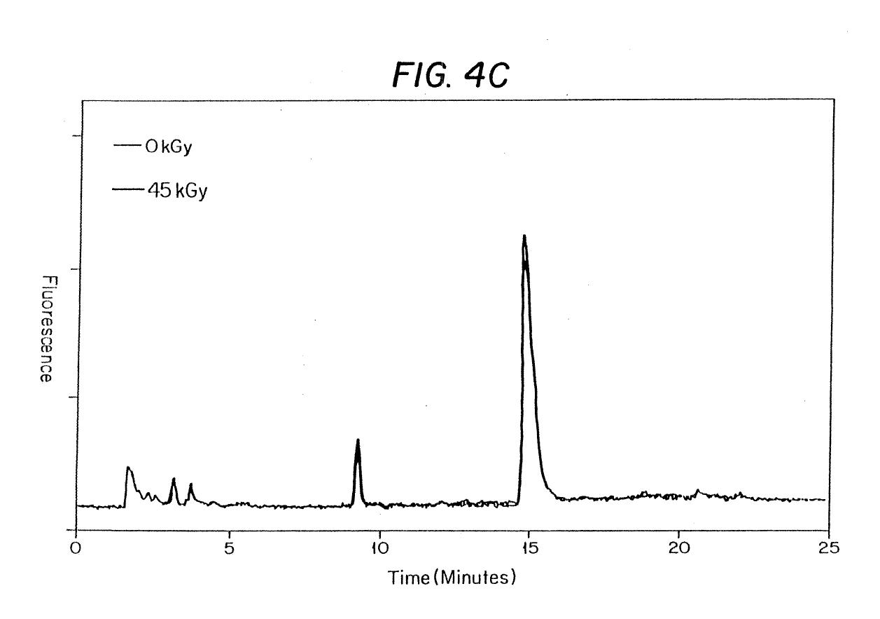

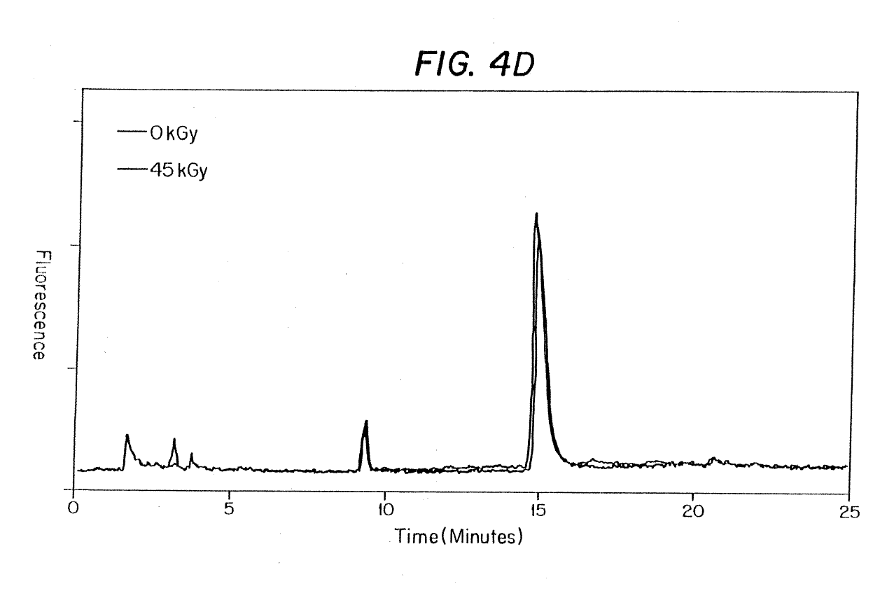

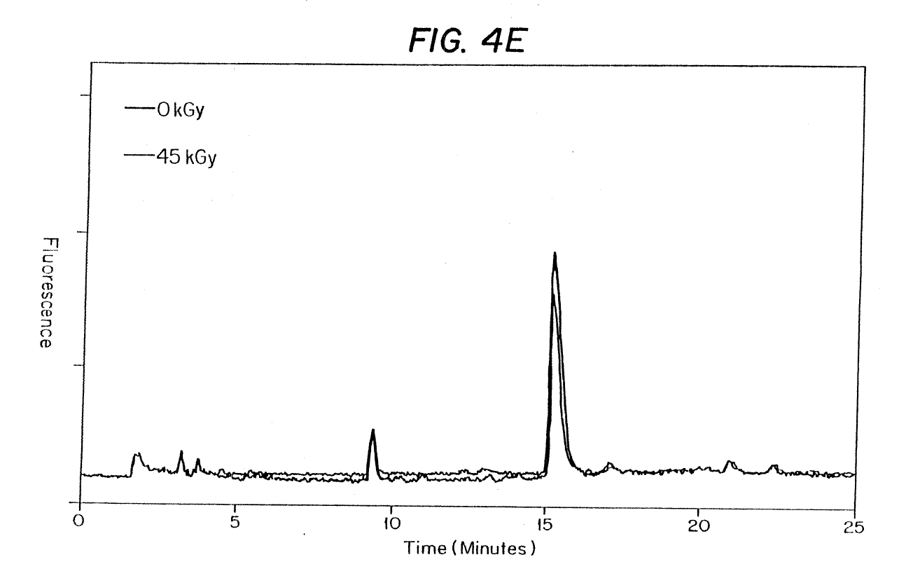

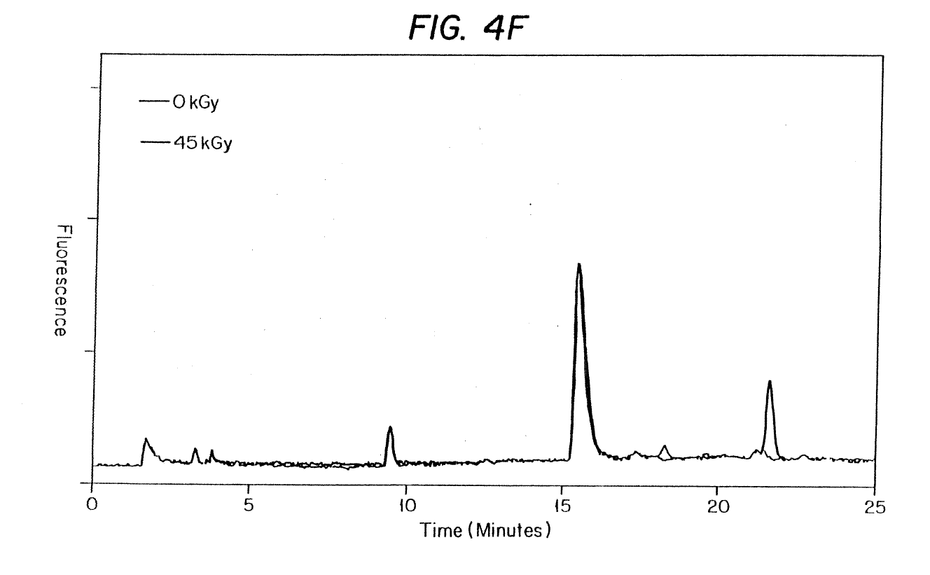

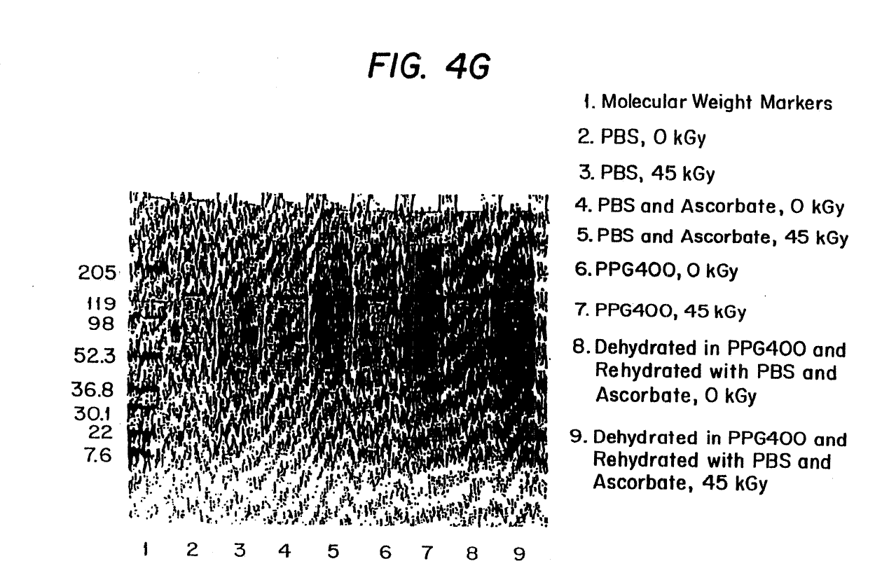

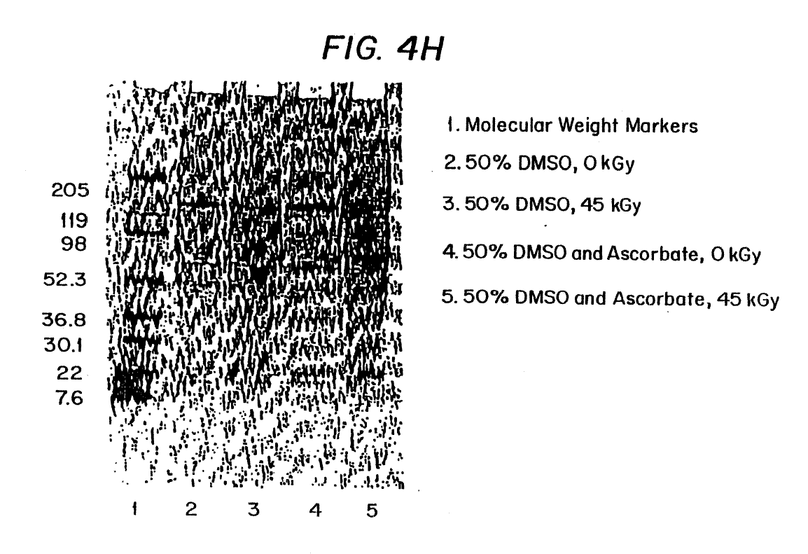

The results of the HPLC analysis are shown in FIGS. 4A-4F. Irradiation in an aqueous environment (PBS) resulted in changes in the minor peaks and a right shift in the major peak. The inclusion of various non-aqueous solvents, reduction in residual water, and the addition of stabilizers produced profiles that more closely matched those of the corresponding controls. The gel analysis is shown in FIGS. 4G-4H and shows a significant loss of bands in PBS, while the other groups demonstrated a significant retention of these lost bands.

When comparing the results from Example 4 to the results from Examples 1, 2, and 3, it becomes apparent that lowering the temperature for the gamma irradiation usually results in a decrease in the amount of modification or damage to the collagen crosslinks. One illustration of this temperature dependence is the sample containing 50% DMSO and ascorbate, in which the additional peaks are markedly decreased as the temperature is lowered from −20° C. to −80° C. It is also clear that reducing residual water content by replacing it with a non-aqueous solvent results in less damage or modification, as does adding the stabilizers shown.

### Example 5

In this experiment, the protective effect of the absence or presence of a stabilizer cocktail on frozen porcine ACL samples, which were gamma irradiated to a total dose of 45 kGy at approximately 6 kGy/hr at −80° C., was evaluated.

Materials:

1. Porcine ACL samples were obtained and placed in 15% DMSO or 15% DMSO containing 100 mM ascorbate, 100 mM deferoxamine, and 22 mM ergothioneine and incubated for 1 hour at 37° C. with agitation and then at 4° C. for 24 hours.
2. The ACL samples were quick frozen in ethanol, dry-ice bath and then stored at −80° C. until irradiation
   Methods:
1. ACL samples were sent to the irradiator on dry ice.
2. Gamma irradiation was performed at NIST at 5.18 kGy/hour to a total dose of 45 kGy at an average temperature of −75° C. The 0 kGy controls were maintained on dry ice.
3. Irradiated samples were as follows:
   a. 4 M Guanidine-0.5 M sodium acetate, pH 5.8 extraction and SDS-PAGE;
   b. Pepsinolysis of guanidine residue and SDS-PAGE;
   c. CNBr digest of pepsin residue and SDS-PAGE;
   d. SDS-PAGE of CNBr digest residue; and
   e. Hydrolysis and evaluation of pyridinoline crosslinks by HPLC.
      Results:

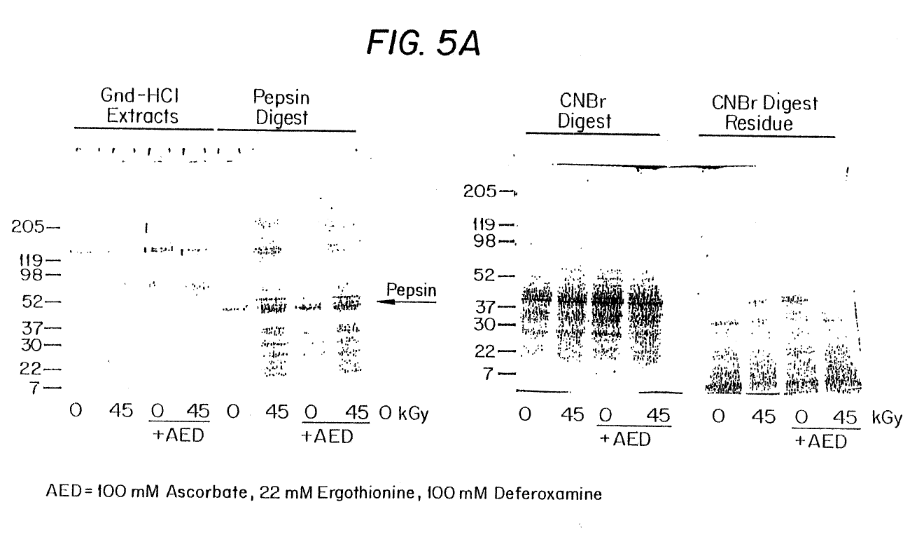

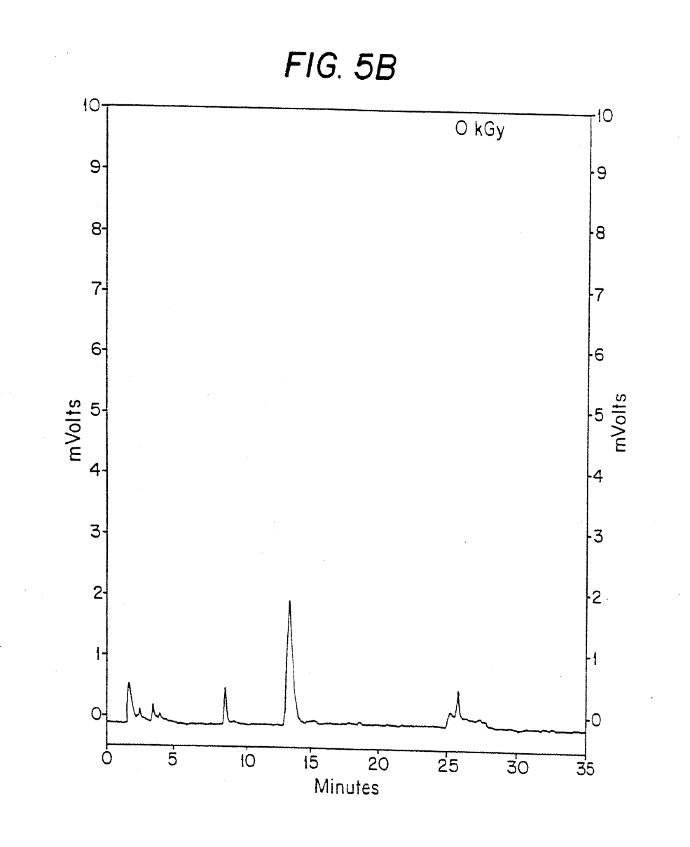

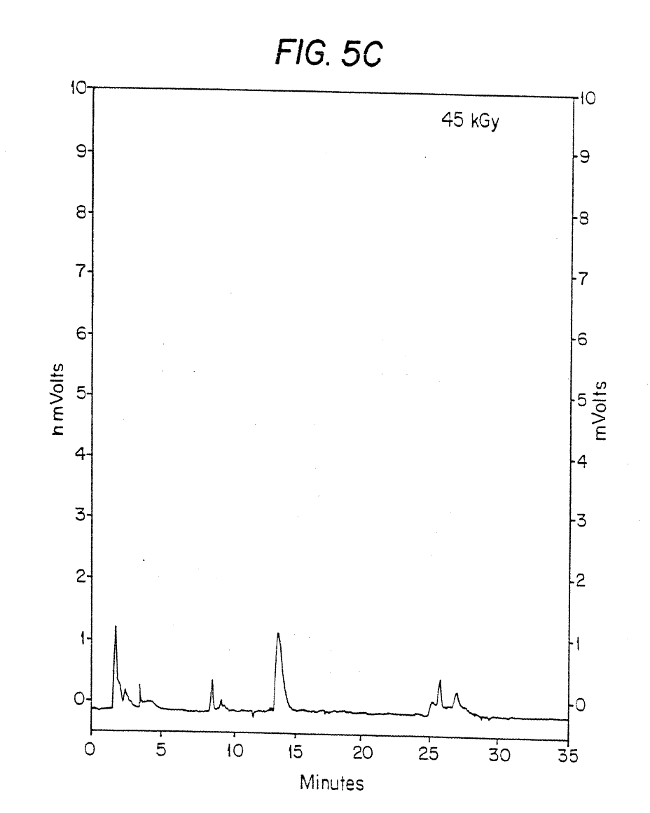

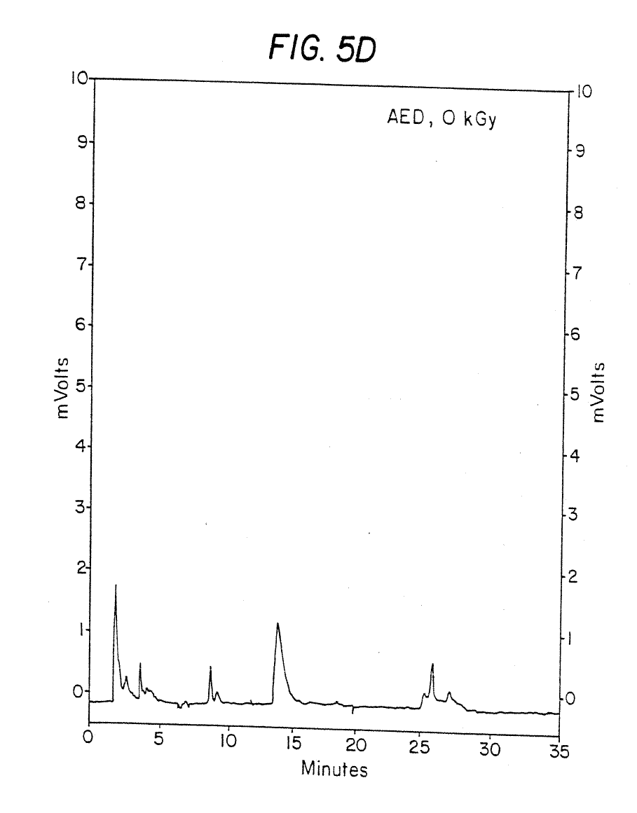

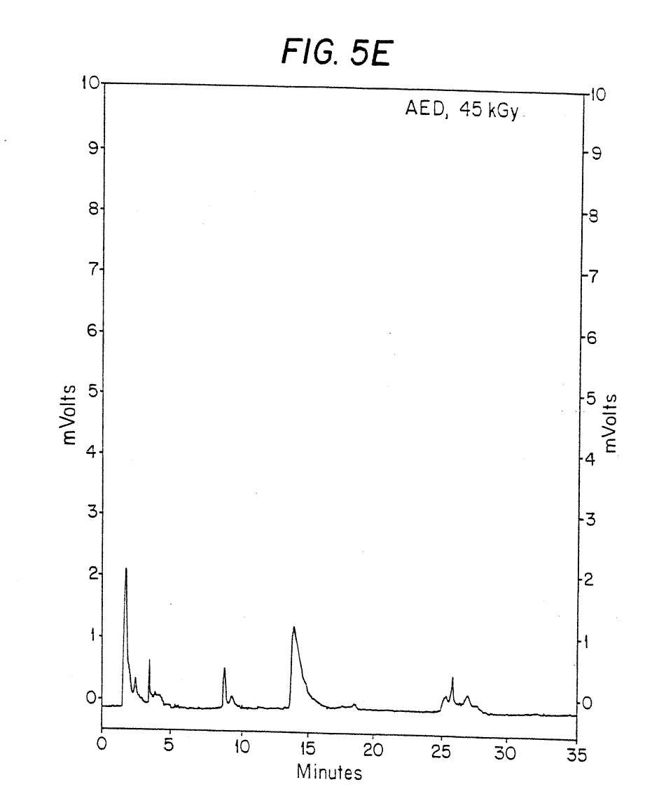

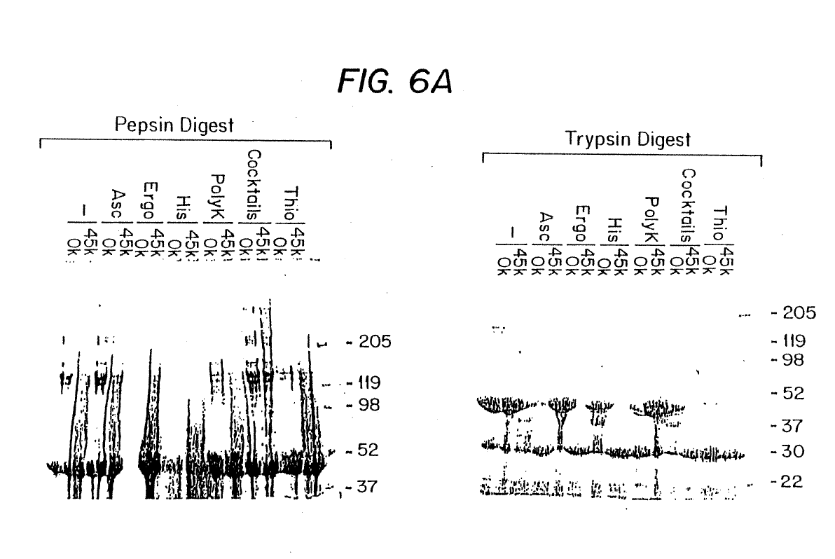

As illustrated in FIG. 5A, fewer proteins overall were extracted by guanidine/acetate following irradiation to 45 kGy, and of those that were extracted, there was significantly less protein in the 45 kGy sample than the control sample subjected to 0 kGy of irradiation. Additionally, also in FIG. 5A, there are a series of bands around 205 kD that are absent from the 5 kGy sample. The top two of the four bands were detected, however, in the 45 kGy sample with the cocktail. There are three darker staining bands that run just above the 119 kD marker, the top band of which appears to be sensitive to gamma irradiation. Additionally, there are a series of bands around 205 kD that are absent from the 45 kGy sample.

Also, as illustrated in FIG. 5A, the SDS-PAGE analysis of the pepsin-solubilized component of the guanidine/acetate residue indicates that more material was extracted by pepsinolysis following 45 kGy of gamma irradiation compared to the 0 kGy controls. There also appeared to be a significant difference between the 0 and 45 kGy samples in the region of 52 to 119 kD. Additionally, there is evidence of increased smearing and higher molecular weight material that does not enter the gel in the 45 kGy sample lanes. There also does not seem to be a gross difference between the 45 kGy samples with or without the cocktail.

Further, as illustrated in FIG. 5A, no differences appear among the samples following CNBr cleavage of the residue left after pepsin digestion. As illustrated in FIGS. 5B-5E, HPLC analysis of the Pyridinoline crosslinks indicates that there is about a 20% loss in crosslink of the 45 kGy samples compared to the 0 kGy sample. The peak profiles of the samples containing cocktail are broader and there appears to be a loss of symmetry. The cocktail or ratio of tissue to HCl during may also affect the hydrolysis.

Pretreatment of the ACL tissue with the AED stabilizer cocktail provided minimal protection to radiation-induced damage. SDS-PAGE of the guanidine extracted material indicated that several higher molecular weight proteins are sensitive to gamma irradiation and therefore might serve as markers for later evaluation.

### Example 6

In this experiment, the effect of gamma irradiation on frozen porcine ACL samples soaked in the absence or presence of a stabilizer was evaluated

Materials:

Porcine ACL samples with the following stabilizers were prepared:
a. 200 mM sodium ascorbate (Spectrum S1329 QP 0839) in water;
b. 100 mM thiourea (Sigma T8656, 11K01781) in water;
c. 200 mM L-histidine (Sigma H8776, 69H1251) in PBS;
d. 500 mM D(+)-trehalose (Sigma T9531, 61K7026) in water;
e. 5 mg/mL ergothionine (Sigma E7521, 21K1683) in water;
f. 0.01 M poly-Lysine (Sigma, MW 461);
g. PPG for 1 hour at 37° C., then removed and soaked in a PPG cocktail of 100 μM trolox C (Aldrich 23,881-3, 02507TS, 53188-07-01) in DPBS, 100 mM lipoic acid (Calbiochem 437692, B34484), 100 mM coumeric acid (Sigma) in ethyl alcohol and 100 mM n-propyl gallate (Sigma P3130, 60K0877) in ethyl alcohol; and
h. No stabilizers added (water only).
   Methods:
1. ACL samples were prepared by cutting each sample in half in the longitudinal direction;
2. Porcine ACL samples were obtained and placed in one of the stabilizers for 1 hour in a shaking incubator at 37° C.;
3. Next, the samples were dehydrated for 1 hour at 37° C. in PPG 400;
4. The samples were then placed at 4° C. with the stabilizer previously used for an additional 1 hour, and then fresh stabilizers were added and soaking occurred for 3 days at 4° C. Then the samples were decanted and freeze dried. Fresh stabilizers were also added prior to freeze drying.
5. ACL samples were freeze dried, then gamma irradiation was performed at NIST with 0 and 45 kGy of gamma irradiation at 1.677 kGy/hr.
6. Irradiated samples were as follows:
   a. Control (ACL) in water;
   b. ACL+200 mM sodium ascorbate, pH 7.63;
   c. ACL+100 mM thiourea, pH 6.63;
   d. ACL+200 mM L-histidine, pH 8.24;
   e. ACL+500 mM trehalose, pH 5.24;
   f. ACL+5 mg/mL ergothionine, pH 6.0;
   g. ACL+0.01 M poly-Lysine, pH 5.59; and
   h. ACL dehydrated+PPG cocktail (100 μM trolox C, 100 mM lipoic acid, 100 mM coumeric acid and 100 mM n-propyl gallate), pH 5.24.
7. Guanidine HCL extraction was done with 4 M GuHCl in 0.5 NaOAC pH 5.8 and 5 mM EDTA, 10 mM NEM, 5 mM Benzamidine and 1 mM PMSF to a final concentration of 100 mg/ml of wet tissue weight/ml of extraction buffer. The samples were incubated at 4° C. on a nutator for 2 days.
8. Pepsin digestion was done by first centrifuging these extracts, then transferring the remaining pellets into a 2 ml tube. The pellets were then washed 3 times with 0.5 M HOAC. Pepsin was added at 1:10 of enzyme:tissue in 0.5 M HOAC and incubated at 4° C. overnight.
9. For pepsin-digested supernatant, NaCl form 5 M stock solution was added to a final concentration of 1M. The supernatants were centrifuged and collagen gel pellets were resuspended in 1 ml of 0.5 M HOAC with gentle mixing at 4° C.
10. Performed DEAE chromatography on dialysates of Guanidine extracts of samples. Eluants from the DEAE column were subjected to SDS-PAGE and visualized by staining with Toluidine Blue.
11. A BCA assay was performed on the dialysates of the PPG+cocktail guanidine extracted samples to determine the total protein concentration in the samples.
12. Extracted PPG+cocktail treated samples using Urea/SDS/β-Me extraction buffer. The extractible noncollagenous proteins were analyzed by SDS-PAGE under reducing conditions.
   Results:

The ACL samples were rehydrated with water for a few hours at room temperature, where a measured length of each ligament was cut and weighed. The weights of the cut pieces is as follows:

Sample

| | 0 kGy (mg) | 45 kGy (mg) |
|---|---|---|
| No stabilizer | 134.5 | 150.45 |
| sodium ascorbate | 171.95 | 148 |
| thiourea | 288.6 | 183.06 |
| L-histidine | 229.3 | 226.54 |
| D(+)-trehalose | 260 | 197.5 |
| ergothionine | 165.14 | 132.68 |
| poly-Lysine | 289.34 | 164.88 |
| PPG cocktail | 114.5 | 83.93 |

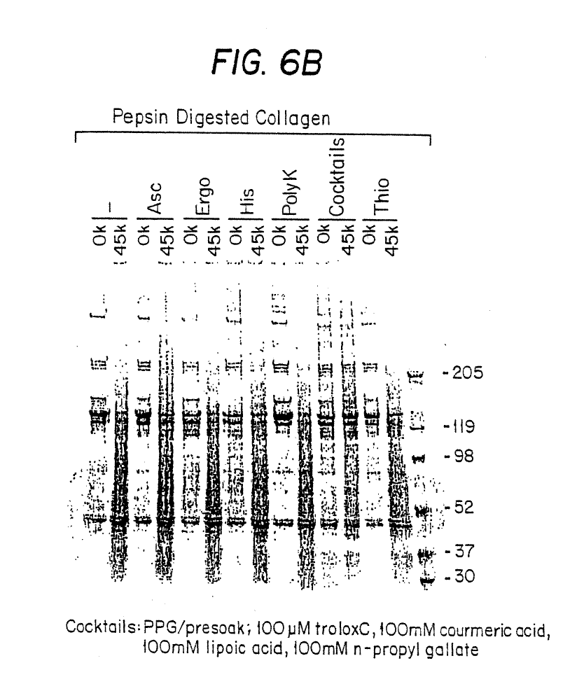

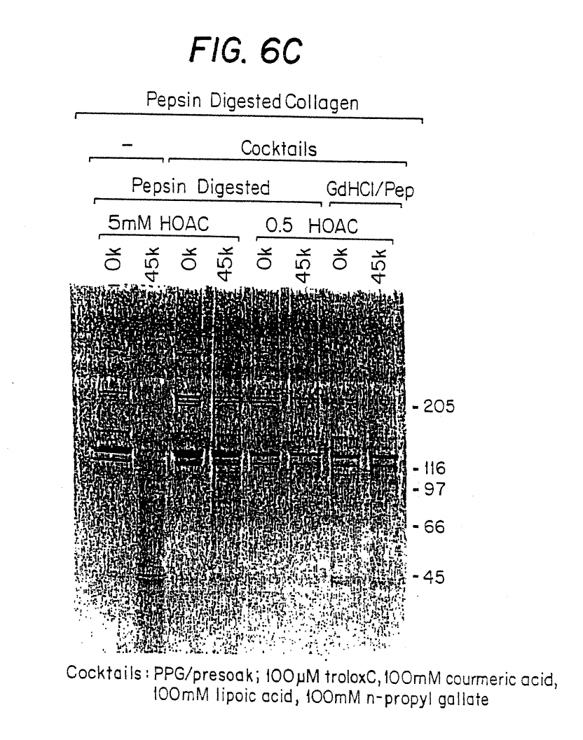

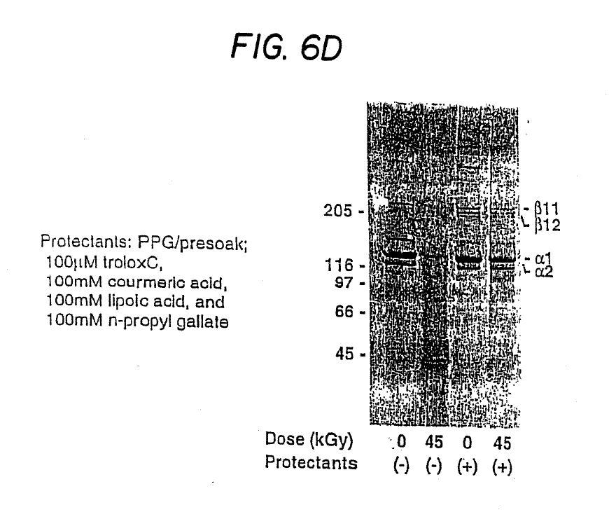

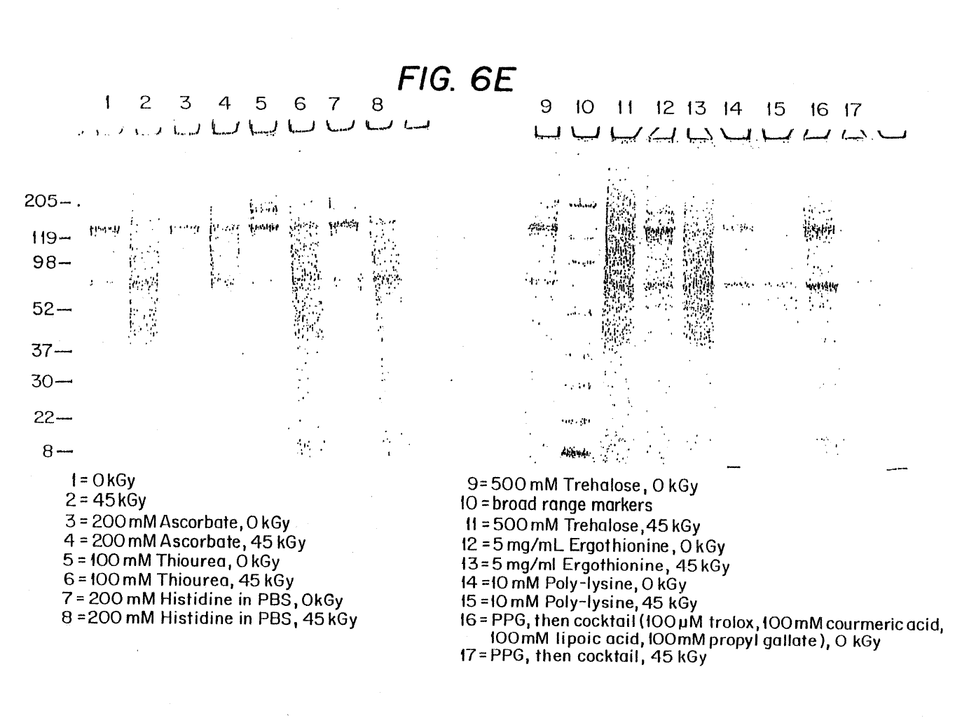

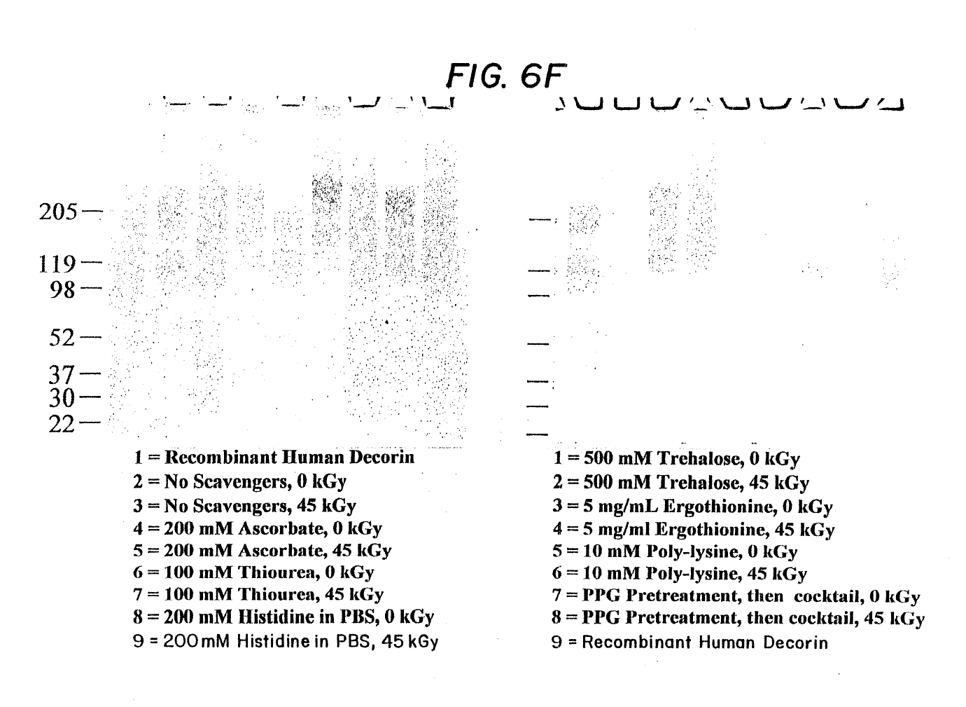

From the SDS-PAGE of pepsin digest, the cocktail treated ACL showed the best recovery compared to the other stabilizers. The HMW bands, as illustrated in FIG. 6A, were protected after irradiation in the presence of the cocktail mix.

For the purified pepsin-digested collagen, the PPG dehydration and rehydration with cocktail showed the best recovery by SDS-PAGE. The yield, as illustrated in FIGS. 6B, 6C and 6D was about 88% for the cocktails comparing to 32% for the control. However, some of the HMW bands were destroyed by irradiation even in the presence of the cocktails. These other stabilizers were not effective in protecting the collagen in this experiment.

The turbidity of the collagen appeared to be lower in the presence of the cocktail with a lower rate of fibril formation compared to the un-irradiated collagen.

SDS-PAGE of the guanidine extracts, as illustrated in FIG. 6E indicate severe damage to the extractable proteins following irradiation to 45 kGy as compared to the corresponding 0 kGy control The addition of the various stabilizers gave variable results. The 0 kGy controls differed from one another which either reflects the efficiency of their extraction in the presence of the various stabilizers or is an artifact of the dialysis. Trehalose and polylysine provided the least protection. Ascorbate and histidine provided the most promising results for protecting a broad spectrum of the proteins, while ergothionine showed good protection of proteins in the lower ⅔ of the gel. The cocktail provided protection to the proteins in the region above the 119 kD marker. However, the very high molecular weight proteins were not well preserved by any of the stabilizers.

Using DEAE chromatography, as illustrated in FIG. 6F, the proteoglycan profile appeared varied and inconsistent from sample to sample and from control to control. It is unclear whether the stabilizers were affected. It is clear, however, that there is a high molecular weight proteoglycan (>200 kD) that was purified in several of the samples. Most of the samples had a band that migrated similar to that of the recombinant human decorin. However, it is not clear whether it is porcine decorin.

Using BCA and SDS-PAGE on the PPG+Cocktail sample, guanidine extracts were evaluated based on SDS-PAGE of equal protein load. The protein concentrations were as follows:

| | |
|---|---|
| fdL/PPG + C/0 | 1270 ng/μL |
| fdL/PPG + C/45 | 249 ng/μL |

Although there appears to be significantly less protein in the 45 kGy sample based on concentration alone, there appears to be a similar amount of total protein when the volume was taken into consideration where the 45 kGy sample appears diluted. Additionally, the SDS-PAGE analysis shows loss of specific protein bands with other bands appearing to be less sensitive to radiation. Densitometry was performed on two different protein bands, as follows:

| | | |
|---|---|---|
| background | 4.52 | |
| 0 kGy | 50.26 | |
| 45 kGy | 26.87 | percent of 0 kGy: 53.5% |
| background | 2.33 | |
| 0 kGy | 70.26 | |
| 45 kGy | 50.54 | percent of 0 kGy: 71.9% |

It is appears that the different recoveries observed are due to differences in sensitivity to radiation or due to a difference in extraction ability. For example, the loss observed in the 45 kGy sample might be due to a differential loss (i.e. —damage) of the proteins or might be due to radiation-induced cross linking that results in a different ability of various proteins to be extracted.

Using Urea/SDS/β-Me extraction the initial difference in guanidine extraction of the PPG+cocktail samples can be observed. It appears that the PPG+cocktail treatment resulted in significant protection of the extractible proteins at 45 kGy of gamma irradiation compared to the 45 kGy sample without treatment. However, it is noted that the PPG+cocktail sample did not rehydrate, but the lack of rehydration appears to be irradiation independent and therefore caused by some component or combination of components in the treatment, which was investigated in Example 7, as follows.

### Example 7

In this experiment, the protective effect of the PPG+cocktail treatment of Example 6 was observed to determine whether the ACL sample was adversely affected due to the lack of rehydration.

Materials:

1. α-Lipoic Acid (Calbiochem #437692, lot B34484);
2. Trolox C (Aldrich #23,881-3, lot 02507TS);
3. n-Propyl Gallate (Sigma #P-3130, lot 60K0877);
4. p-Coumaric Acid (Sigma #C-9008, lot 49H3600);
5. Polypropylene Glycol P400 (Fluka #81350, lot 386716/1);
6. 5 mL tubes;
7. left ACL (received from RadTag Technologies);
8. Ethyl Alcohol (Burdick & Jackson, #AH090-4, lot BX488)
   Methods:
1. The ACL samples was sectioned and dehydrated in PPG for 2 hours @37° C. with shaking.
2. Components of the stabilizer cocktail were made individually by making the stocks, then diluting them with 40% ethanol (which alone does not prevent rehydration of the tissue), where the individual stabilizers/controls were as follows:
   a. 2 mM Trolox C in PBS (diluted 1:20 in 40% ethanol, final of 100 μM)
   b. 1 M propyl gallate dissolved in ethanol (diluted 1:10, final of 100 mM in 40% ethanol)
   c. 0.5 M coumaric acid in ethanol (diluted 1:5, final 100 mM in 40% ethanol)
   d. 0.5 M lipoic acid initially dissolved in NaOh and then the volume and pH were adjusted to neutral (diluted 1:5 in 40% ethanol)
   e. 40% ethanol
   f. water
3. Following a 2 hour incubation in PPG, the tissue was removed and blotted to remove excess PPG and 2 mL of the individual stabilizers/controls (a-f) were added.
4. Samples were then placed on a shaker at 4° C. and allowed to rehydrate overnight.
   Results:

The ACL tissue samples were rehydrated to a normal appearance except the sample treated with PPG and coumaric acid. The coumaric acid was then tested without the PPG, but still did not result in a normal process by rehydration and instead led to adverse properties of the ACL tissue sample which appeared dehydrated and sticky to the touch.

### Example 8

In this experiment, the protective effect of a cryopreservative on a gamma irradiated regulated or quick freeze dried ACL at −80° C. was evaluated.

Materials:

1. Edmonton cryopreservative media (M199, 10% FCS, Penicillin-Streptomyocin, 2 M DMSO)
2. Modified VS55 cryoprotectant (100 mM trehalose, 15 mM KH2PO₄, 42 mM K₂HPO₄, 15 mM KCl, 10 mM NaHCO3, 150 mM mannitol, 24.2% DMSO, 16.8% 1,2-propanediol, 14% formamide). See U.S. Pat. No. 6,194,137 B1.
3. 200 mM sodium ascorbate
   Methods:
1. ACL samples were submerged in either the Edmonton or VS 55 media.
2. Samples were frozen by reducing the temperature 1° C. per minute to −40° C. in the freeze dryer and then placing the samples at −80° C. (regulated freeze) or freezing in a dry ice-ethanol bath (quick freeze).
3. Irradiations were performed at NIST on dry ice using 5.2 kGy/h to a total dose of 50 kGy.
4. The following analyses were performed:
   a. Gnd-HCL extraction and SDS-PAGE;
   b. Urea/SDS/β-Me extraction and SDS-PAGE;
   c. Collagenase digestion of Gnd-HCL residue and SDS-PAGE;
   d. Collagen purification and SDS-PAGE; and
   e. DEAE chromatography and SDS-PAGE.
      Results:

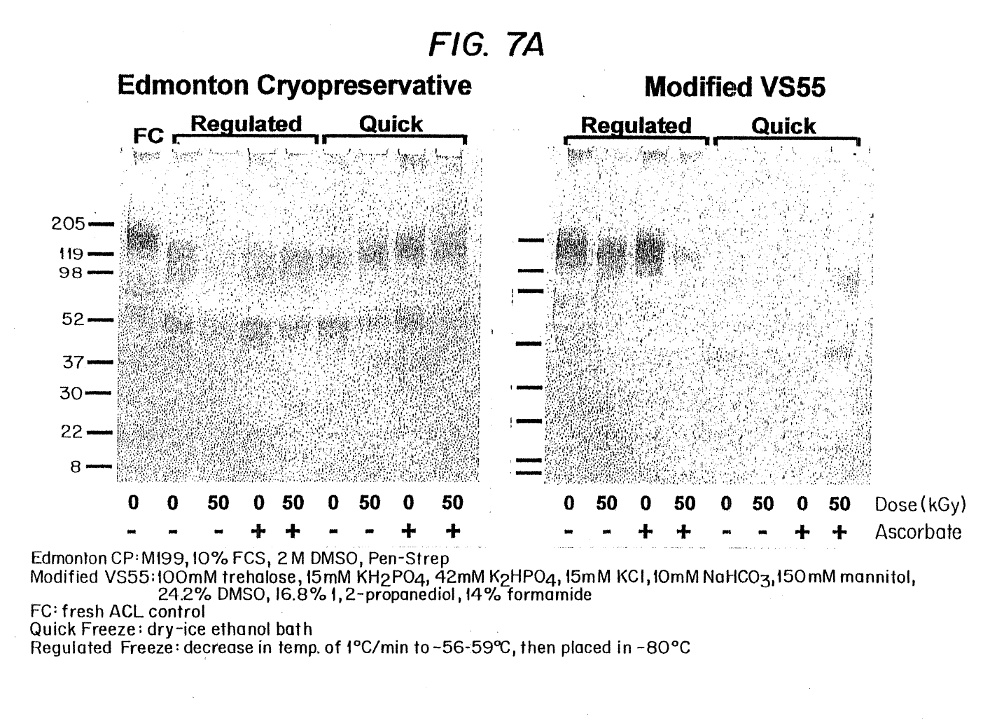

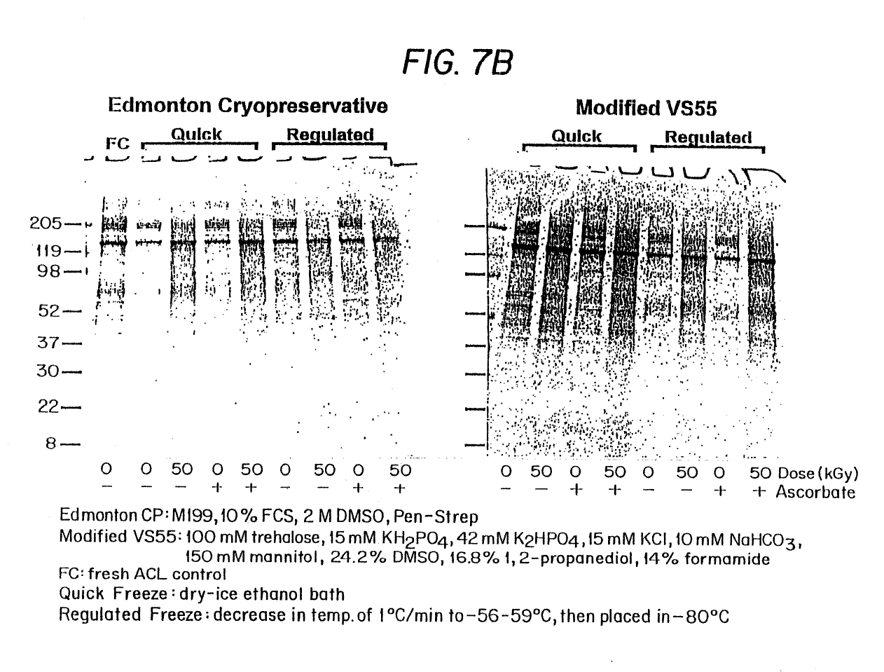

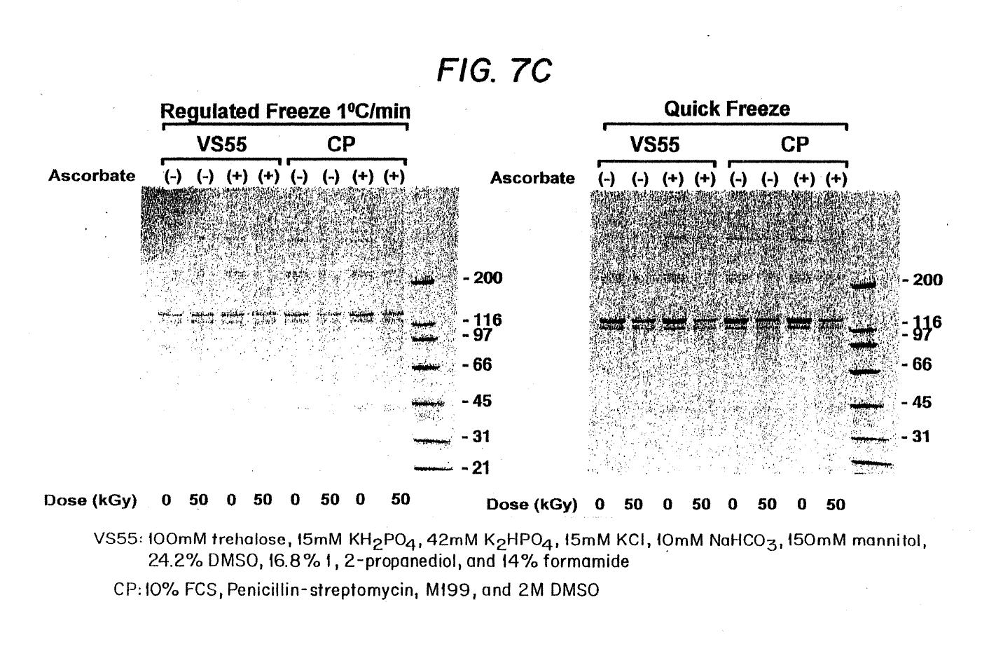

Purification of proteoglycans by DEAE chromatography appeared to show that the cryopreservative treatment influenced the ability of the proteoglycans to be purified, as illustrated in FIG. 7A. Also as illustrated in FIG. 7A, all samples submerged in Edmonton CP had a similar profile, but varied in intensity. On the other hand, as further illustrated in FIG. 7A, treatment with VS55 gave poor recovery of proteoglycans under the quick freezing regimen, whereas the regulated freeze resulted in good recovery except in the sample containing ascorbate.

A table of the percent recovery of the major band observed by SDS-PAGE, comparing the irradiated sample to its corresponding control for the guanidine extracts, is given below; For the samples treated with CP, those samples in which 200 mM ascorbate was added, had a lower percent recovery than the sample without ascorbate. And, the quick freeze gave better recovery than the regulated freeze. Whereas, with the mVS55 treated samples the regulated freeze had better recovery based on the densitometry of single band. However, by visual examination, the overall total protein extracted from the regulated freeze appeared to be less than that extracted from the quick freeze. Additionally, the exaggerated percent recoveries (>100%) are likely an artifact of smearing and the absence of some of the higher molecular weight proteins. However, the mVS55 does seem to give better recovery of these high molecular weight proteins (around 205 kDa) in the irradiated samples than other irradiated samples without mVS55.

The gels of the Urea/SDS/β-Me extractible proteins appear to be consistent with the results observed with the guanidine extraction, FIG. 7B. Densitometry was not performed on these samples as the smearing observed in the irradiated samples leads to inaccurate readings. To that end, the obvious presence of the smearing indicates damage to tissue proteins following irradiation.

Major Band

| Edmonton CP | | | | Modified VS55 | | | |
|---|---|---|---|---|---|---|---|
| | Dens. | Blank Sub. | % Recovery | | Dens. | Blank Sub. | % Recovery |
| Quick Freeze | | | | Quick Freeze | | | |
| Blank | 27.71 | 0 | | Blank | 45.91 | 0 | |
| 0 kGy | 137.3 | 109.59 | 100 | 0 kGy | 161.68 | 115.77 | 100 |
| 50 kGy | 138.75 | 111.04 | 101 | 50 kGy | 161.52 | 115.61 | 100 |
| Asc. 0 kGy | 137.05 | 109.34 | 100 | Asc. 0 kGy | 166.56 | 120.65 | 100 |
| Asc. 45 kGy | 122.75 | 95.04 | 87 | Asc. 45 kGy | 151.4 | 105.49 | 87 |
| Regulated Freeze | | | | Regulated Freeze | | | |
| Blank | 27.71 | 0 | | Blank | 40.07 | 0 | |
| 0 kGy | 135.98 | 108.27 | 100 | 0 kGy | 126.39 | 86.32 | 100 |
| 50 kGy | 104.54 | 76.83 | 71 | 50 kGy | 139.27 | 99.2 | 115 |
| Asc. 0 kGy | 137.14 | 109.43 | 100 | Asc. 0 kGy | 128.19 | 88.12 | 100 |
| Asc. 45 kGy | 95.79 | 68.08 | 62 | Asc. 45 kGy | 152.55 | 112.48 | 128 |

From the SDS-PAGE, purified pepsin-digested collagen from the VS55 cryopreservatives without ascorbate showed the best recovery, as illustrated in FIG. 7C and in the following table:

| Regulated Freezing | | | | Quick Freeze | | | |
|---|---|---|---|---|---|---|---|
| Density of a chain Collagen | | | % Recovery | Density of a chain Collagen | | | % Recovery |
| Blk | 15.01 | 0 | | Blk | 50.99 | 0 | |
| VS55/0kGy | 46.39 | 31.38 | 100 | VS55/0kGy | 112.45 | 61.46 | 100 |
| VS55/50kGy | 51.72 | 36.71 | 117 | VS55/50kGy | 100.75 | 49.76 | 81 |
| VS/A/0kGy | 53.53 | 38.52 | 100 | VS/A/0kGy | 117.61 | 66.62 | 100 |
| VS/A/50kGy | 42.79 | 27.78 | 72 | VS/A/50kGy | 88.7 | 37.71 | 57 |
| CP/0kGy | 58.7 | 43.76 | 100 | CP/0kGy | 112.36 | 61.37 | 100 |
| CP/50kGy | 43.92 | 28.91 | 66 | CP/50kGy | 86.21 | 35.22 | 57 |
| CP/A/0kGy | 80.98 | 65.97 | 100 | CP/A/0kGy | 122.36 | 71.37 | 100 |
| CP/A/50kGy | 56.02 | 41.01 | 62 | CP/A/50kGy | 87.42 | 36.43 | 51 |

Turbidity results for pepsin-digested collagen from ACL in VS55 cryopreservative did not correlate well with the SDS-PAGE data for regulated freeze and quick freeze ACL samples. The collagen from irradiated ACL in VS55 did not form fibril as expected, probably due to the presence of degraded proteins and loss of high molecular weight protein bands after irradiation (which interfere with the assay). For other cryopreservatives turbidity results correlated quite well with the SDS-PAGE results for quick freeze and regulated freeze ACL samples.

### Example 9

This experiment was to determine whether ethanol dehydration or drying ACL will help to remove water and whether a rehydration process would deliver cocktail of antioxidants inside ACL tissue to protect it from γ-irradiation at 4° C. with 50 kGy.

Materials:

1. 2 mM trolox C [Aldrich 23,881-3, 02507TS, 53188-07-01] in DPBS

2. 0.5M lipoic acid [Calbiochem 437692, B34484] in 100% ethanol

3. 0.5 M coumeric acid [Sigma C4400] in ethyl alcohol

4. 1M n-propyl gallate [Sigma P3130, 60K0877] in ethyl alcohol

5. 10 mg/ml Ergothionine [Sigma E7521, 21K1683] in water.

Samples were prepared by cutting ACL in small chunk and used for irradiation as following:

1. Control (ACL)

2. Cocktails (100 μM troloxC, 100 mM coumeric acid, 100 mM lipoic acid, 100 mM n-propyl gallate)

3. Cocktails+5 mg/ml ergothionine.

Methods:

1. Six pieces of ACL were dried overnight to remove water.

2. Another six pieces were soaked in 25% ethanol for 2 hr at room temperature (rt), then 50% ethanol for 1 hr at rt and 75% ethanol for overnight at rt.

3. Soaked another 6 pieces of ACL in 100% ethanol for 6 hr at rt and these ACLs were incubated with either cocktails or modified cocktails solutions for 2 hr with shaking in a shaking incubator at 37° C. After 2 hr incubation, these ACL tubes were decanted and fresh solution of anti-oxidants were added to each ACL containing tubes and incubated for overnight at 4° C.

4. All the tubes were freeze-dried for 2 days.

5. The samples were irradiated with 0 and 50 kGy at 1.656 kGy/hr at NIST.

6. The ligaments were rehydrated with water for a few hours at rt.

7. Washed extensively with DPBS.

8. For ethanol dehydration ACL samples, rehydration was repeated by washing with the gradient of 75°/, 50%, and 25% ethanol. Then washed with DPBS extensively.

9. Cut a small piece from each sample and weighed all of the cut pieces.

| | | |
|---|---|---|
| a) | ETOH | 0 kGy = 25.12 mg |
| | | 45k = 10.5 mg |
| b) | ETOH/Cocktails | 0 kGy = 25.6 mg |
| | | 45 kGy = 32.4 mg |
| c) | ETOH/modified | 0 kGy = 30.45 mg |
| | | 45 kGy = 30.3 mg |
| d) | FD | 0 kGy = 30.1 mg |
| | | 45 kGy = 16.3 mg |
| e) | FD/cocktails | 0 kGy = 33.3 mg |
| | | 45 kGy = 31.51 mg |
| f) | FD/modified | 0 kGy = 30 mg |
| | | 45 kGy = 26.5 mg |

10. ACLs were digested with pepsin and collagen was purified by salt precipitation.

11. Collagen gel pellets were resuspended in 1 ml of 0.5 N HOAC with gently mixing at 4° C.

12. The pepsin-digested collagens for control and cocktails treated ACL were dialyzed against 5 mM HOAC for overnight.

13. Determined the OD 218 nm for each collagen preparation.

14. Turbidity assay was performed for these collagens.

Results:

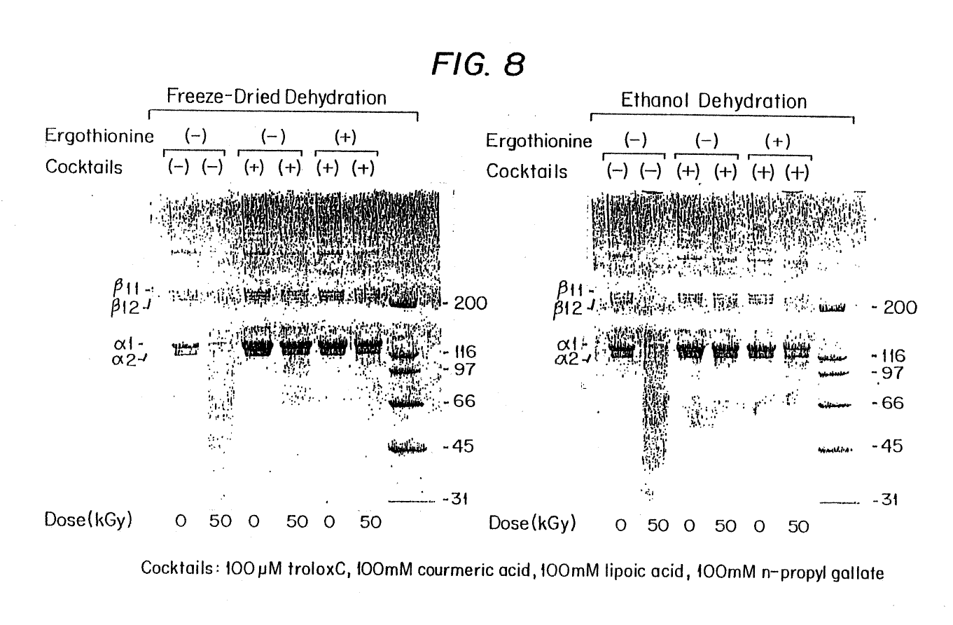

The purified pepsin-digested collagen for ethanol dehydration of ACL with cocktails without ergothionine, as illustrated in FIG. 8, showed the best recovery compared with cocktails with ergothionine by SDS-PAGE. The yield was 88% for the cocktails with ethanol dehydration comparing to 83% for freeze-died dehydration. The cocktails of scavengers and ergothionine was a little less effective than that of cocktails alone. However, some of the HMW bands (possible chain of collagen) were still destroyed by irradiation.

Ethanol dehydration seemed to give a little bit better recovery than the freeze-dried dehydration process for ACL.

### Example 10

This experiment was to determine whether high salt, low salt, neutral pH and low pH treated ACL will help to deliver stabilizers into ACL tissue to protect it from γ-irradiation at −80° C. with 50 kGy.

Methods:

1. Prepared stock solution 2M sodium ascorbate (Spectrum S1349, Lot#QP0839) in water. Samples were prepared with the following:
a) DPBS
b) DPBS/200 mM sodium ascorbate
c) 0.5N HOAC
d) 0.5N HOAC/200 mM sodium ascorbate
e) 20 mM sodium phosphate pH 7.6
f) 20 mM sodium phosphate pH 7.6/200 mM sodium ascorbate
g) 20 mM sodium phosphate pH 7.6/1M NaCl
h) 20 mM sodium phosphate pH 7.6/1M NaCl/200 mM sodium ascorbate.

2. These samples were irradiated with 0 and 50 kGy at 1.53 kGy/hr at NIST.

3. A small piece was cut from each sample and weighed as follows:

| | | |
|---|---|---|
| a) | DPBS | 0 kGy = 32.7 mg |
| | | 45k = 10.7 mg |
| b) | DPBS/Asc | 0 kGy = 25.12 mg |
| | | 45 kGy = 26 mg |
| c) | 0.5N HOAC | 0 kGy = 37.3 mg |
| | | 45 kGy = 35.5 mg |
| d) | 0.5N HOAC/Asc | 0 kGy = 21.2 mg |
| | | 45 kGy = 41.4 mg |
| e) | 20 mM PO4 | 0 kGy = 22.87 mg |
| | | 45 kGy = 36.3 mg |
| f) | 20 mM PO4/Asc | 0 kGy = 24 mg |
| | | 45 kGy = 18.04 mg |
| g) | 20 mM PO4/NaCl | 0 kGy = 21.41 mg |
| | | 45 kGy = 21.2 mg |
| h) | 20 mM PO4/NaCI/Asc | 0 kGy = 33.76 mg |
| | | 45 kGy = 21 mg |

4. ACL were digested with pepsin and collagen purified by precipitating with salt.

Results:

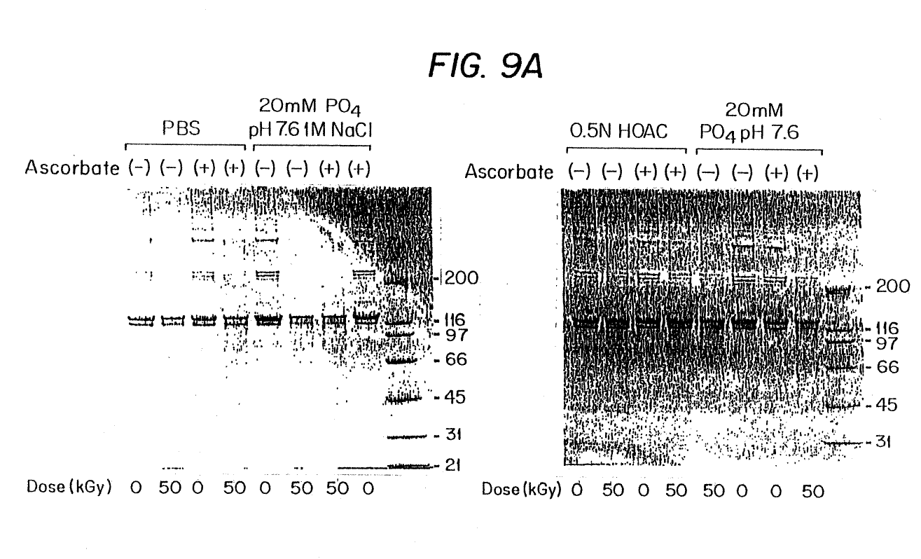

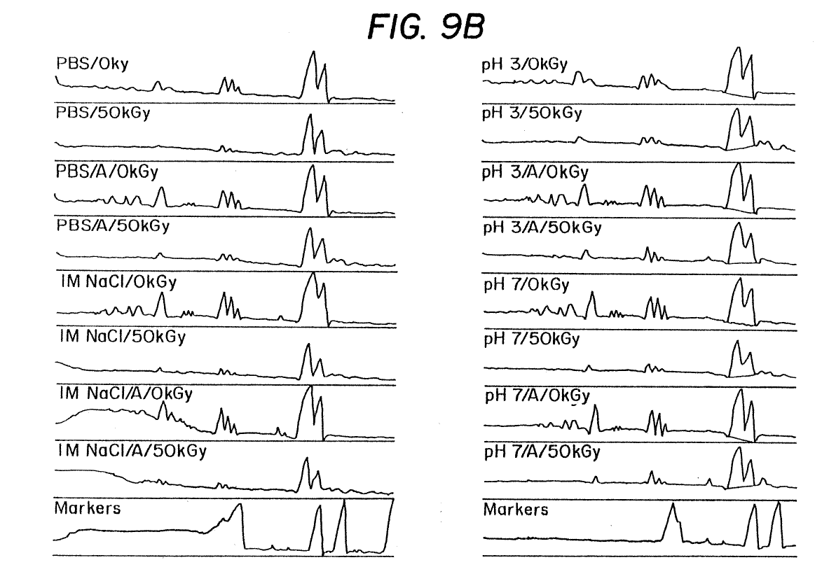

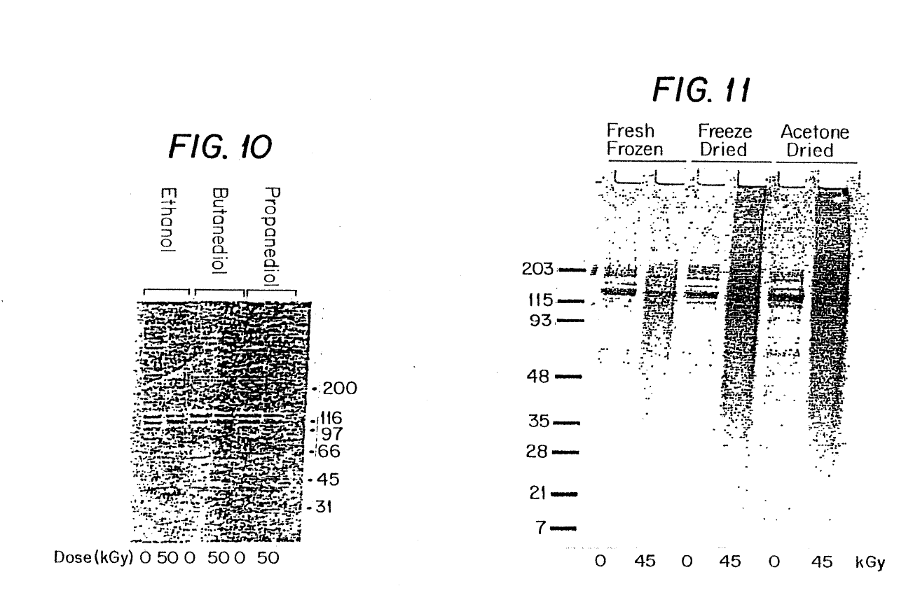

The purified pepsin-digested collagen from ACL irradiated at −80° C. with 0.5N HOAC pH 3.4, as illustrated in FIGS. 9A and 9B, showed the best recovery compared with 20 mM sodium phosphate pH 7.6 with or without 1M NaCl or PBS alone by SDS-PAGE. The yield at 50 kGy was 83% with ascorbate and 73% without ascorbate. ACL irradiated with 20 mM sodium phosphate pH 7.6 without salt yielded good recovery at 75% and 60% in the presence and absence of ascorbate, respectively. ACL irradiated with high salt showed the worst recovery only 40% with or without ascorbate. Some of the HMW bands (possible y chain of collagen) were still destroyed by irradiation.

The turbidity assay appeared to have the collagen isolated from the ACL samples. Also, the washing of the collagen gel pellet after salt precipitation seemed to help. Collagen isolated from ACL irradiated with 0.5N HOAC showed the best results, which correlated with SDS PAGE results. However, the turbidity curves of collagens from ACL irradiated in the presence of ascorbate did not quite correlate with SDS PAGE results, which showed better recovery than that of ACL irradiated under conditions without ascorbate, which may be caused because the ascorbate may not have been completely removed from the ACL sample.

Also, it appeared that the ACL sample soaking with 0.5N HOAC caused the tissue to swell and become larger than its original size. After washing with DPBS, however, the tissue appeared to change back to its original size.

### Example 11

This experiment was to determine whether alcohols can protect ACL tissue samples from γ-irradiation at −80° C. with 50 kGy.

Methods:

1. ACL samples were prepared by preparing small portions of ACL sample with the following:

| | |
|---|---|
| a) | ethanol |
| b) | 1,2-propanediol |
| c) | 2,3-butanediol |

2. These samples were then incubated with different alcohols for 2 hr in a shaking incubator at 37° C.

3. After 2 hr incubation, these ACL tubes were decanted and fresh solutions were added to each ACL containing tubes and incubated overnight at −80° C.

4. These samples were irradiated with 0 and 50 kGy at 1.53 kGy/hr at NIST.

5. These ligaments were washed extensively with DPBS. Small pieces from each sample were cut, then weighed as follows:

| | | |
|---|---|---|
| a) | DPBS | 0 kGy = 22.9 mg |
| | | 50k = 16.43 mg |
| b) | DPBS/Asc | 0 kGy = 47.1 mg |
| | | 50 kGy = 21.85 mg |
| c) | 0.5N HOAC | 0 kGy = 32.5 mg |
| | | 50 kGy = 30.8 mg |

6. ACL were digested with pepsin and collagen purified by precipitating with salt.

7. Turbidity assay was performed for these collagens using at [1 mg/ml].

8. Ran 10 μg of each purified pepsin-digested collages on 4-12% gel and quantified both alpha 1 and alpha 2 chains.

Results:

The purified pepsin-digested collagen from ACL irradiated at −80° C. with ethanol or butanediol showed good recovery, as illustrated in FIG. 10. The yields for 50 kGy ACL collagen were 77% and 88% based on the densitometry of alpha 1 and 2 chains of collagen, respectively. Some of the HMW bands (possible β and γ chains of collagen) were completely destroyed by irradiation. Although the recoveries were good, the recovery of collagen isolated from ACL irradiated in the presence of 20 mM P04 and ascorbate was still better.

A turbidity assay was performed for the collagen isolated from these ACL samples. Correlation was found between the ACL collagen before and after irradiation. Collagen isolated from ACL irradiated in the presence of alcohol and propanediol could not form fibrils even at higher collagen concentration 0.5 mg/ml comparing to normal used 0.25 mg/ml concentration.

### Example 12

This experiment was to compare the effects of gamma irradiation on ACL samples that were subjected to three different types of preservation: fresh frozen, freeze dried, or solvent-dried, as these methods of preservation are used by various tissue banks/processors.

Method:

1. Tissue cross sections were sliced and weighed.

| | | |
|---|---|---|
| a. | acl/fresh/−80/0 | 330.0 g |
| b. | acl/fresh/−80/45 | 335.9 mg |
| c. | acl/fd/−80/0 | 286.2 mg |
| d. | acl/fd/−80/45 | 272.4 mg |
| e. | acl/ad/−80/0 | 298.9 mg |
| f. | acl/ad/−80/45 | 274.3 mg |

2. Fresh ligaments were placed in 2 mL serum vials and frozen in a dry ice-ethanol bath and then stored in a −80° C. freezer until irradiation.

3. The freeze-dried ligaments (fd) were placed in 2 mL serum vials for freeze drying. The freeze dried tissue was then stored in a −80° C. freezer until irradiation.

4. The acetone-dried ligaments were placed in 5 mL conical vials and 5 mL acetone was added. The samples were placed at 4° C. on the nutator. The acetone was changed every hour for 4 hours and the 5th acetone wash went overnight. The next morning the samples were removed from the acetone and blotted dry with a Kimwipe. The dried ligaments were placed in a 2 mL serum vial and the residual acetone was allowed to evaporate in a hood overnight. The acetone-dried ligament appeared to be dehydrated and shriveled. The samples were stored in the −80° C. freezer until irradiation.

5. All samples were irradiated at NIST to 45 kGy on dry ice (−72° C.) at 1.5 kGy/h. The 0 kGy controls traveled and were stored on dry ice at NIST.

6. Rehydrated tissue with 2 mL PBS for 1.5 h at 4° C. with shaking on the Nutator.
a. All looked rehydrated except for the acetone-dried tissues that still appeared shriveled and hard to the touch.
b. Transferred tissues to conical vials with 20 mL PBS and left overnight at 4° C. with shaking on the Nutator.
c. All tissues rehydrated.

7. Extracted noncollagenous protein with Urea/SDS/B-Me extraction buffer. Analyzed samples by SDS-PAGE (4-20% gradient) under reducing conditions.

8. Pyd-cross link recovery was determined.

Results:

Gamma irradiating ACL's to 45 kGy at low temperature, as illustrated in FIG. 11, resulted in better recovery than irradiating freeze-dried ACL's to 45 kGy at 4° C. In addition, the freeze-dried sample irradiated to 45 kGy in this study resulted in a better recovery of noncollagenous proteins than was observed for the freeze-dried 45-kGy-sample irradiated at 4° C.

This study indicates that irradiating fresh frozen tissue yields better recovery of the noncollagenous proteins than is observed when the tissue has been dehydrated by freeze drying or solvent drying (acetone) prior to irradiating as indicated by the extensive smearing observed on the gel. Densitometry indicated that the major band seen on the gel was similar in all the 0 kGy controls; however, percent recovery with the corresponding 45-kGy samples could not be performed due to smearing, which results in an exaggerated densitometry reading and a high reading artifact.

### Example 13

In this experiment, the effects of gamma irradiation an porcine ACL treated with various stabilizers was investigated.

Preparation of Antioxidant Stock Solutions

The following stock solutions were prepared:

2M sodium ascorbate in water (Spectrum S1349 QP 0839)

2 mM trolox C in DPBS (Aldrich 23,881-3, 02507TS, 53188-07-01)

0.5M lipoic acid (Calbiochem 437692, B34484)

0.5M coumaric acid in ethyl alcohol (Sigma)

1M n-propyl gallate in ethyl alcohol (Sigma P3130, 60K0877)

0.2M L-histidine in PBS (Sigma H8776, 69H1251)

2M D-(+)-trehalose in water (Sigma T9531, 61K7026)

10 mg/ml ergothionine in water (Sigma E7521, 21K1683)

0.04M poly-lysine (Sigma, MW=461)

1M thiourea (Sigma T8656, I 1K01781)

Preparation of Ligament Samples

Samples were prepared by cutting ACL in half longitudinally. The lengths of each ACL were measured and used for irradiation. The samples were placed in tubes with the following conditions:

1. ACL in water (Control)

2. ACL+200 mM sodium ascorbate, pH 7.63

3. ACL+0.1M thiourea, pH 6.64

4. ACL+200 mM histidine, pH 8.24

5. ACL+500 mM trehalose, pH 5.36

6. ACL+5 mg/ml ergothionine, pH 6.0

7. ACL+0.01M poly-lysine, pH 5.59

8. ACL dehydrated+(100 μM trolox C, 100 mM coumaric acid, 100 mM lipoic acid, 100 mMn-propyl gallate), pH 5.24

Method

ACL's 1-7 described above were incubated for about 1 to about 2 hours with shaking in a shaking incubator at 37° C. For the dehydration (8), the ACL was incubated with polypropylene glycol 400 (PPG400) for 1 hour at 37° C. The PPG400 treated ACL was incubated with the antioxidant mixture described above for 1 hour at 37° C. After about 2 hours of incubation, the ACL tubes were decanted and fresh solutions of antioxidants, or water for 1, were added to each ACL tube. Following this, the tubes ACL's were incubated for 3 days at 4° C., decanted and freeze-dried.

The samples were irradiated with 0 kGy and 45 kGy at 1.677 kGy/hr.

The samples were rehydrated with water for a few hours at room temperature. The length of the ACL's was measured and a small piece was cut from each irradiated ACL. The cut pieces were weighed with the following results:

| Sample Number | 0 kGy (mg) | 45 kGy (mg) |
|---|---|---|
| 1 | 134.5 | 150.45 |
| 2 | 171.95 | 148 |
| 3 | 288.6 | 183.06 |
| 4 | 229.3 | 226.54 |
| 5 | 260 | 197.5 |
| 6 | 165.14 | 132.68 |
| 7 | 289.34 | 164.88 |
| 8 | 114.5 | 83.93 |

Guanidine CHI Extraction

The ACL samples were extracted with 4M GuHCl in 0.5M NaOac, pH 5.8, and 5 mM EDTA, 10 mM NEM, 5 mM benzamidine and 1 mM PMSF for a final concentration of 100 mg/ml or wet tissue weight/ml of extraction buffer. The samples were incubated on the nutator for 2 days at 4° C.

Following incubation, the extracts were centrifuged using a tabletop centrifuge and the pellets were transferred into 2 ml tubes and washed 3 times with 2 ml of 0.5M HOAC. Pepsin was added to the pellets at a 1:10 ratio of enzyme to tissue in 0.5N HOAC. The samples were incubated at 4° C. overnight and another portion of pepsin was added to each pellet. The samples were incubated on the nutator at 4° C. overnight.

The samples were centrifuged and washed 3 times with 100 mM Tris, pH 8.0, and 20 mM CaCl₂. Trypsin was added at a 1:20 ratio of enzyme to wet weight. The samples were mixed and incubated at 37° C. overnight.

To the pepsin-digested supernatant, NaCl from 5M stock solution was added to a final concentration of 1M. The supernatants were centrifuged for 15 minutes at 22,000 g in a cold room. Collagen gel pellets were resuspended in 1 ml of 0.5N HOAC with gentle mixing at 4° C.

The pepsin digested collagens for the samples were dialyzed against 5 mM HOAC overnight. Determined the OD 218 nm for each collagen preparation. A turbidity assay was performed for these collagens using purified pepsin-digested collagen as a control.

Results

From the SDS-PAGE of the pepsin digest, the antioxidant cocktail treated ACL (8) showed the best recovery compared to other antioxidants. The HMW bands were protected after irradiation in the presence of cocktails. The trypsin digest did not provide any conclusive results.

For the purified pepsin-digested collagen, the PPG dehydration and rehydration with scavenger cocktails showed the best recovery by SDS-PAGE. They yield was 88% for the cocktails compared to 32% for the control (1). Some of the HMW bands were destroyed by irradiation even in the presence of scavenger cocktails. These other scavengers were not effective protecting the collagen in this experiment. One possible explanation is that the scavengers were not absorbed deep inside the ACL, since the ACL's were simply soaked with these scavengers.

The turbidity test assay was not working well for the collagen isolated from these ACL. There could be some other proteins interfering with the assay. However, these collagens could from fibrils. The irradiated collagen in the presence of cocktail scavengers has a lower final turbidity and smaller rate of fibril formation compared to the unirradiated collagen.

Using PPG400 for dehydration of the ACL irreversibly changed the morphology of the ACL, even after rehydration.

### Example 14

### Method

Samples of human bone powder were gamma irradiated to a total dose of 20 kGy at rates of 0.19, 5 and 30 kGy/hr on dry ice. A fourth control sample was not irradiated. After irradiation, the three samples and control were ground to 75-500 μm particle size and demineralized by decalcifying for 10 hours in 10% formic acid. The ground samples were extracted with guanidine hydrochloride and 5 μg total protein from each extraction were assayed by RP-HPLC.

Results

As the rate of irradiation increased, there was an increase in the amount of collagen breakdown products.

### Example 15

Samples of human bone were gamma irradiated at dose rates of 0.2 or 0.6 kGy/hr to total doses of 30, 40 or 50 kGy. Following irradiation, the samples were ground and demineralised for 48 hours in 10% formic acid. The osteoinductive activity was measured for each sample using a conventional in vitro osteoinductive bioassay. The demineralised bone powder was added to plates containing cell cultures. At 5 and 15 days these cells were examined for the appearance of newly formed bone. The results are summarized in the following table

| Total Dose, kGy | Dose Rate, kGy/hr | Osteoinductive Activity |
|---|---|---|
| 30 | 0.2 | Good |
| 40 | 0.2 | Good |
| 50 | 0.2 | Poor |
| 30 | 0.6 | Poor |
| 40 | 0.6 | Poor |
| 50 | 0.6 | Poor |

### Example 16

Samples containing 400 mg of demineralised human allograft tissue and 0.04 ml porcine parvovirus were gamma irradiated to a total dose of 0, 30, 40 or 50 kGy. The dose response for viral inactivation of the porcine parvovirus was determined. The results are summarized in the following table:

| Sample No. | Total Dose, kGy | Remaining Titer log₁₀ |
|---|---|---|
| 1 | 0 | 5.03 |
| 2 | 30 | <1.65 |
| 3 | 40 | <1.65 |
| 4 | 50 | <1.65 |

### Example 17

In this experiment, type I collagen at −20° C., −80° C. or freeze-dried at 4° C. were irradiated with gamma radiation to a total dose of 45 kGy in the presence of various stabilizers.

Materials

The following stock solutions were prepared:

(1) 1M thiourea (Sigma T8656) in water;

(2) 0.5M coumarin (Sigma CC4261) in ethanol;

(3) 0.5M 0-coumaric acid (Sigma C4400) in ethanol;

(4) 0.5M curcumin (Sigma C1386) in ethanol;

(5) 1M L-cysteine (Sigma C6852) in water;

(6) 1M 1,3-dimethyl-2-thiourea (Aldrich 534-13-4) in water;

(7) 1M 2-mercaptoethylamine (Sigma M6500) in water; and

(8) 1M 1,3-dimethylurea (Sigma D6254) in water.

(9) Phosphate buffer solution of 40 mM sodium phosphate and 100 mM NaCl; pH=7.66.

Methods

The following samples were prepared to a final volume of 0.5 ml:

(1) 1 mg/ml collagen in 5 mM acetic acid (control);

(2) 1 mg/ml collagen+0.1M coumaric acid;

(3) 1 mg/ml collagen+5 mM curcumin;

(4) 1 mg/ml collagen+0.1M L-cysteine;

(5) 1 mg/ml collagen+0.1M 1,3-dimethyl-2-thiourea;

(6) 1 mg/ml collagen+0.1M thiourea;

(7) 1 mg/ml collagen+0.1M 2-mercaptoethylamine; and

(8) 1 mg/ml collagen+0.1M 1,3-dimethylurea.

The samples were irradiated as follows:

(1) freeze-dried; temperature: 4.7° C.; dose rate: 1.656 kGy/hr; total dose: 45 kGy;

(2) temperature: −20.5° C.; dose rate: 1.537 kGy/hr; total dose: 45 kGy; and

(3) temperature: 72oC; dose rate: 1.530-1.528 kGy/hr; 45 kGy.

Following irradiation, the samples were analyzed by SDS-PAGE. Additionally, the samples were diluted 1:2 with water to give collagen concentrations of 0.5 mg/ml and a turbidity assay was performed to detect collagen fibril formation. Collagen fibril formation was initiated by adding 100 μl of phosphate buffer solution. The assay was done in triplicate using a microtiter plate reader at 340 nm wavelength.

Results

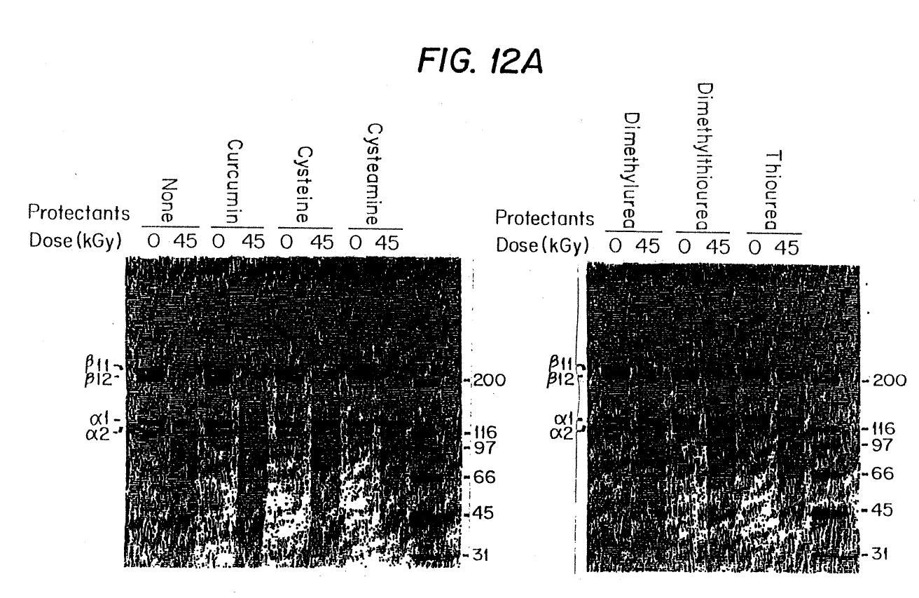

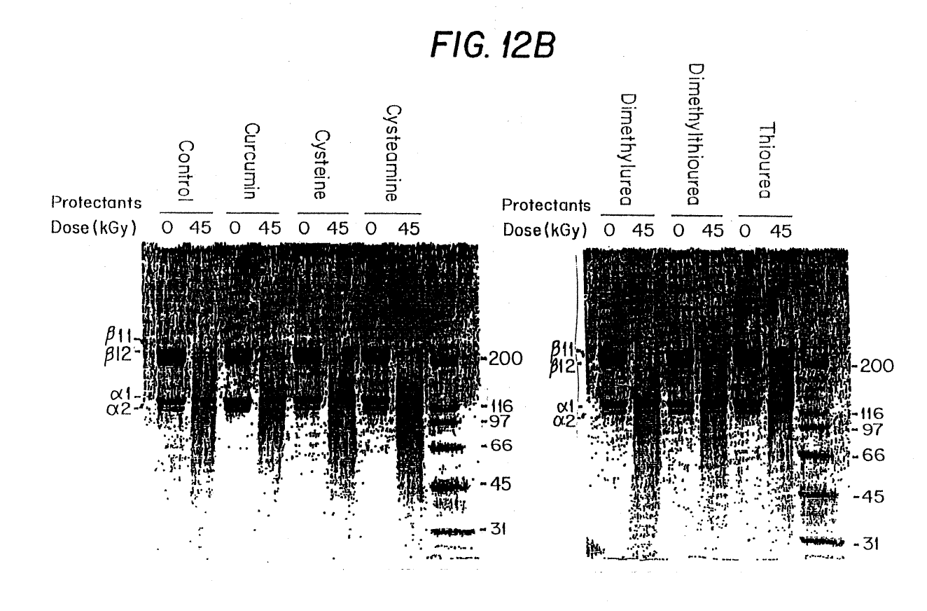

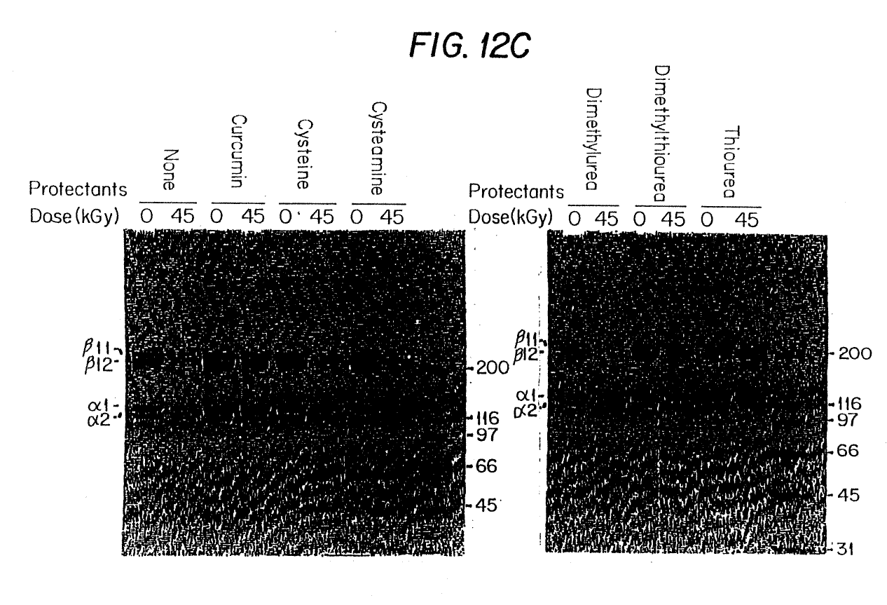

Thiourea and 1,3-dimethyl-2-thiourea protected collagen from gamma irradiation at −20° C., with recoveries of 83 and 86%, respectively. Thiourea and 1,3-dimethyl-2-thiourea also protected the high molecular weight protein bands (possibly gamma chain of collagen). The protective effect of curcumin, cysteine, 2-mercaptoethylamine and 1,2-dimethylurea was less than that observed with thiourea and 1,3-dimethyl-2-thiourea. For the freeze-dried samples irradiated at 4° C., the recoveries for thiourea and 1,3-dimethyl-2-thiourea were 69 and 83%, respectively. Regarding the samples irradiated at −80° C., the recoveries for curcumin, 1,3-dimethyl-2-thiourea and thiourea were 83, 91 and 85%, respectively. FIG. 12A-12C illustrate the SDS-PAGE results.

The turbidity assays showed that samples treated with thiourea and 1,3-dimethyl-2-thiourea could form fibrils after irradiation. Additionally, for the samples irradiated at −80° C., 1,2-dimethylthiourea, thiourea, cysteine and 2-mercaptoethylamine could form fibrils after irradiation.

### Example 18

In this experiment, the effects of gamma irradiation on liquid and gel collagen samples containing various stabilizers were investigated.

Methods

The following stock solutions were prepared:

(1) 2M sodium ascorbate (Spectrum S1349 QP 0839) in water;

(2) 0.25M L-methionine (Sigma M6039 88H11341) in water;

(3) IM Gly-Gly (Sigma G3915 127H54052) in water;

(4) 1M thiourea (Sigma T8656 11k01781) in water; and

(5) Phosphate buffer solution of 40 mM sodium phosphate and 100 mM NaCl; pH=7.66.

The following samples were prepared in duplicate containing either gel or liquid collagen to a final volume of 1 ml by adding 0.5 ml of phosphate buffer solution with 0.5 ml of collagen (1 mg/ml) in the presence of the stabilizer(s) indicated:

(1) Collagen (0.5 mg/ml)+no stabilizer (control);

(2) Collagen (0.5 mg/ml)+50 mM ascorbate;

(3) Collagen (0.5 mg.ml)+50 mM ascorbate+50 mM Gly-Gly;

(4) Collagen (0.5 mg/ml)+25 mM thiourea; and

(5) Collagen (0.5 mg/ml)+25 mM methionine.

For gel samples, after mixing with the phosphate buffer solution the samples were incubated at room temperature for about 30 minutes. The liquid collagen samples were maintained at 4° C. to prevent them from gelling.

The samples were gamma irradiated at about 72° C. (frozen on dry ice) at dose rates of about 1.29-1.41 kGy to a total dose of 48.73 to 53.38 kGy. The irradiated samples were analyzed by SDS-PAGE. Additionally, the samples were diluted 1:2 with water to give collagen concentrations of 0.5 mg/ml and a turbidity assay was performed to detect collagen fibril formation. Collagen fibril formation was initiated by adding 100 μl of phosphate buffer solution. The assay was done in triplicate using a microtiter plate reader at 340 nm wavelength.

Results

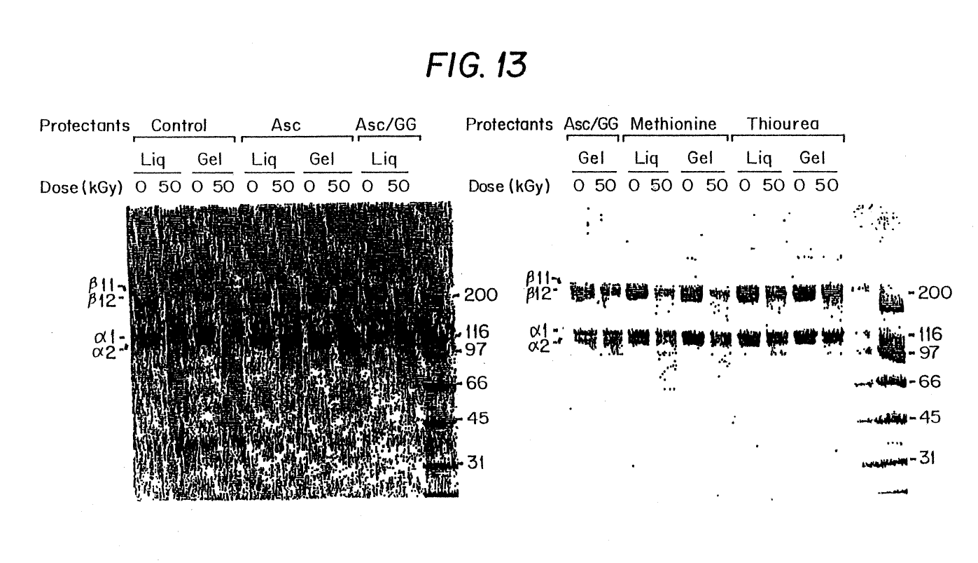

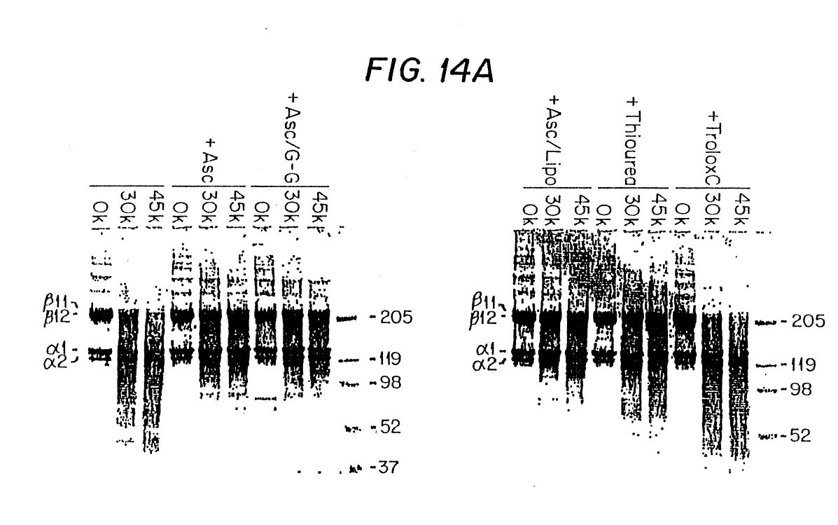

From SDS-PAGE data, FIG. 13, the sample containing the ascorbate/Gly-Gly stabilizer mixture showed the best protective effect for collagen. This stabilizer mixture protected gel collagen more effectively than liquid collagen, with recoveries of 86 and 75%, respectively. Generally, the stabilizers protected gel collagen more effectively than liquid collagen. This may be due the stabilizers being trapped in the gel matrix, thereby being more available to minimize the effects of irradiation.

The turbidity assay results were consistent with the SDS-PAGE analysis. Ascorbate and the ascorbate/Gly-Gly mixture were most effective at protecting gel collagen or liquid collagen.

### Example 19

In this experiment, the effects of gamma irradiation on samples containing collagen and various stabilizers were investigated.

Methods

The following stock solutions were prepared:

(1) 2M sodium ascorbate in water;

(2) 1M Gly-Gly in water;

(3) 2 mM Trolox C in Dulbecco's Phosphate Buffered Saline (DPBS)

(4) 0.5M lipoic acid; and

(5) 1M thiourea in water.

(6) Phosphate buffer solution of 40 mM sodium phosphate and 100 mM NaCl; pH=7.66.

Samples were prepared in duplicate to a final volume of 0.5 ml containing the stabilizer(s) indicated:

(1) Collagen (1 mg/ml) in 5 mM acetic acid (control);

(2) Collagen (1 mg/ml)+200 mM sodium ascorbate;

(3) Collagen (1 mg/ml)+200 mM sodium ascorbate+200 mM Gly-Gly;

(4) Collagen (1 mg/ml)+200 mM sodium ascorbate+200 mM lipoic acid;

(5) Collagen (1 mg/ml)+0.1M thiourea; and

(6) Collagen (1 mg/ml)+200 μM Trolox C

The samples were irradiated as follows:

(1) Liquid; temperature: 3.7° C.; dose rate: 1.67 kGy/hr; total dose: 30 kGy;

(2) Liquid; temperature: −20.3° C.; dose rate: 1.552 kGy/hr; total dose: 30 kGy;

(3) Liquid; temperature: −72.5° C.; dose rate: 5.136 kGy/hr; total dose: 30 kGy;

(4) Liquid; temperature: 3.7 to 5.4° C.; dose rate: 1.67 kGy/hr; total dose: 45 kGy;

(5) Liquid; temperature: −18.6 to −20.3° C.; dose rate 1.552 kGy/hr; total dose: 45 kGy;

(6) Liquid; temperature: −72.5 to −78° C.; dose rate: 5.136 kGy/hr; total dose: 45 kGy;

(7) Freeze dried; temperature: 3.7° C.; dose rate: 1.67 kGy/hr; total dose: 30 kGy; and

(8) Freeze dried; temperature 3.3° C.; dose rate: 1.673 kGy/hr; total dose: 45 kGy.

The samples were analyzed by SDS-PAGE.

Results

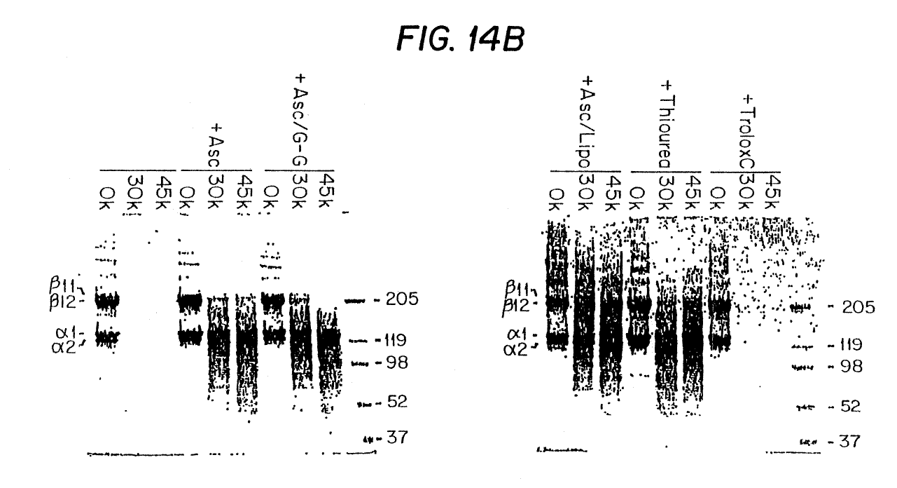

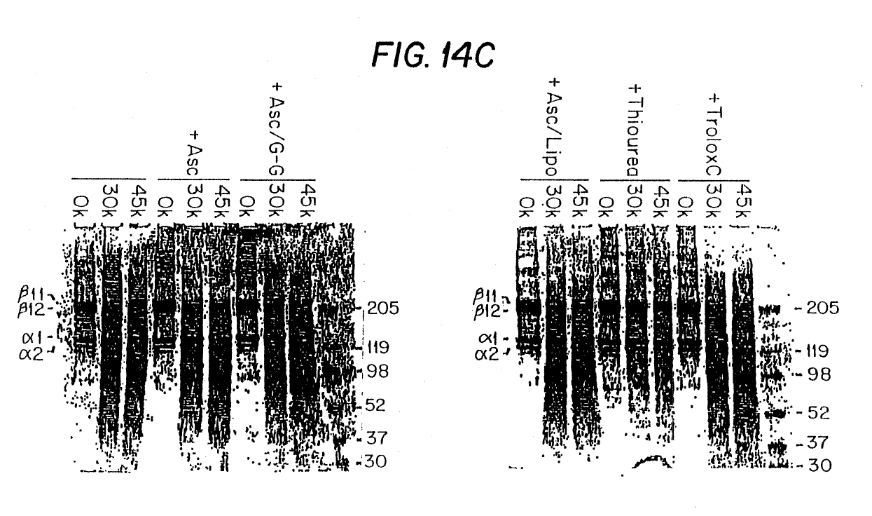

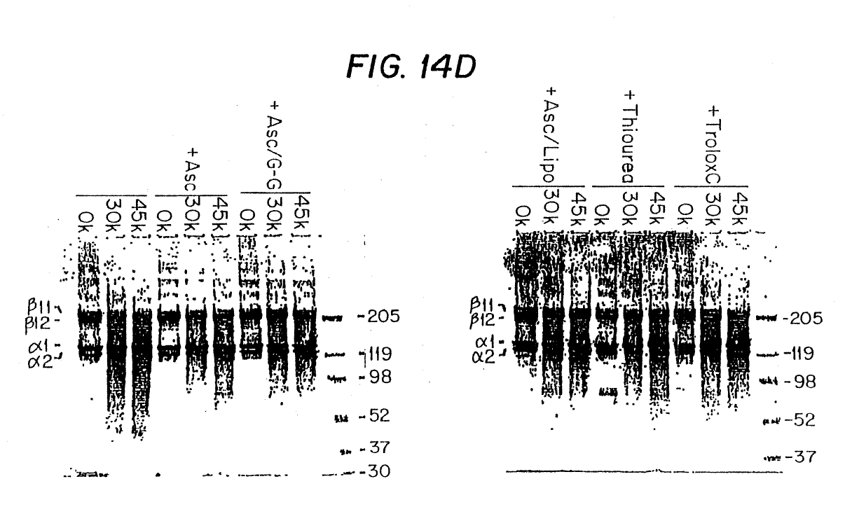

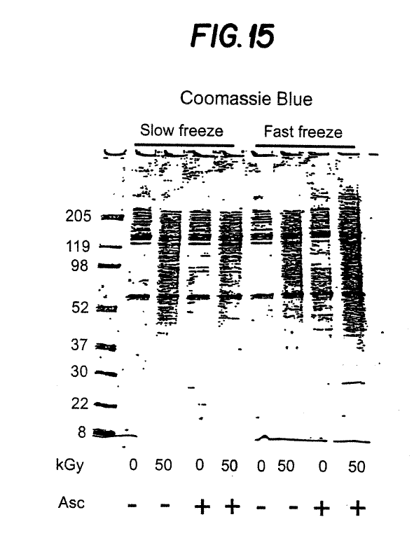

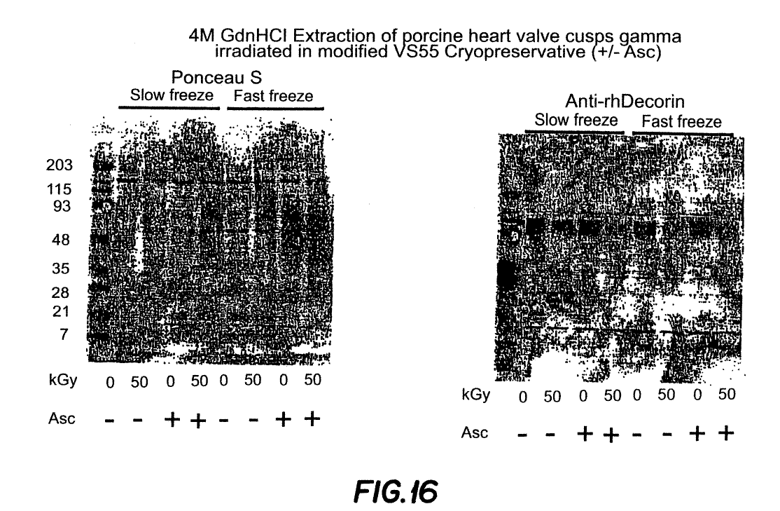

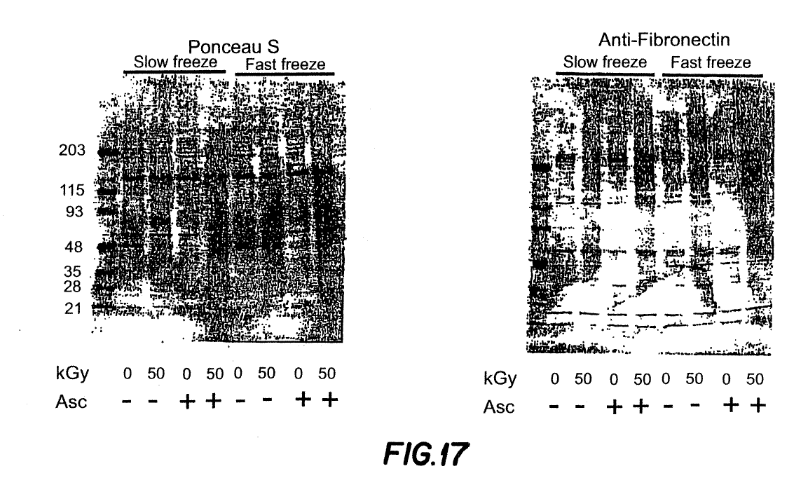

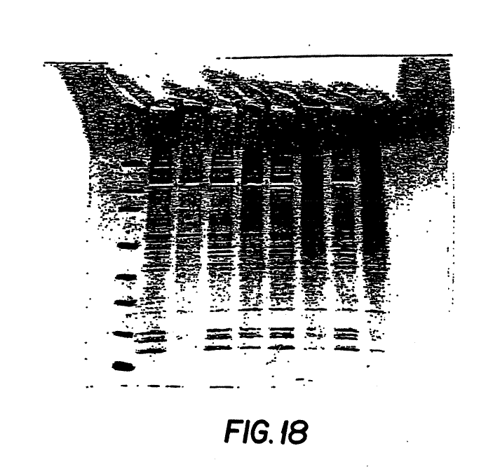

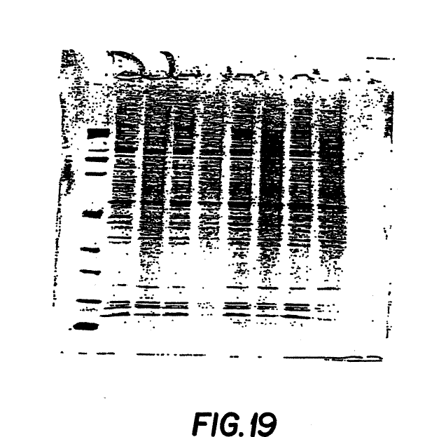

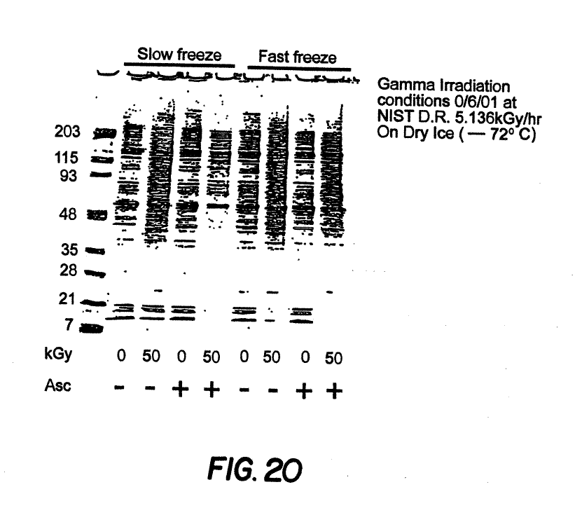

From SDS-PAGE analysis, FIGS. 14A-14D, the samples containing thiourea irradiated to 30 kGy and 45 kGy at about −20° C. had recoveries of 89 and 86%, respectively. Thiourea also protected the high molecular weight protein bands (possibly gamma chain of collagen). The samples irradiated to 30 kGy and 45 kGy at about −20° C. and containing the ascorbate/Gly-Gly stabilizer mixture had recoveries of 81 and 74%, respectively.

Regarding the samples irradiated at about −80° C., those irradiated to a total dose of about 30 kGy and containing thiourea, ascorbate, ascorbate/Gly-Gly, and ascorbate/lipoic acid, showed recoveries of 84, 77, 88 and 86%, respectively. The samples irradiated to a total dose of about 45 kGy had recoveries of 78, 81, 89 and 84%, respectively. The high molecular weight protein bands were also protected by these stabilizers.

Regarding the samples irradiated at about 4° C., for the liquid samples, thiourea appeared to afford the most effective protection. With respect to the freeze dried samples, the samples irradiated to a total dose of about 30 kGy and containing ascorbate, ascorbate/Gly-Gly and ascorbate/lipoic acid had recoveries of 99, 85 and 88% respectively. The samples irradiated to a total dose of about 45 kGy and containing ascorbate, ascorbate/Gly-Gly and ascorbate/lipoic acid had recoveries of 83, 81 and 85% respectively.

### Example 20

### Clostridium V2.doc

In this experiment, the effects of gamma irradiation on Clostridium sordellii in bovine bone was investigated.

Methods

Freeze-dried vials of Clostridium sordellii purchased from ATCC were placed in a bovine bone that contained four holes with a diameter slightly greater than the circumference of the vials that extended to the midpoint of the bone. The bone containing the vials was then irradiated at 1.5 kGy/hr with 0, 25 or 50 kGy of gamma radiation at either 4° C. or on dry ice. The contents of the vials were then resuspended in 10 mL of Reinforced Clostridial Medium supplemented with Oxyrase to provide an anaerobic environment. Serial ten-fold dilutions were made to a dilution of 10⁻⁹. Fifty microliters of each dilution was then spread on a plate containing Reinforced Clostridial Medium plus 1.5% agar. A BBL GasPak Anaerobic System was used to provide an anaerobic environment for growth of the plated bacteria. The broth cultures and the plates were incubated at 37° C. for 48 hours. Following incubation turbidity was visualized and absorbance readings were taken at 620 nm in the broth cultures and colonies were counted on the plates. Similar cultures of Staph. epidermidis and E. coli were also set up and irradiated. These cultures were prepared using media and conditions conventional for the organisms.

Results

Unirradiated tubes of Clostridium sordellii showed frank growth as detected by obvious turbidity at dilutions ranging from the Stock suspension to 10⁻⁸. When exposed to 25 kGy at 4 C, all tubes were clear of growth from 10⁻¹ to 10⁻⁹. Only the undiluted Stock suspension showed signs of growth. When the irradiation dose was increased to 50 kGy, no growth was observed in any of the tubes. Similar results were seen for the materials irradiated on dry ice. These results are shown in the following table:

| | | | Log reduction | Log reduction |
|---|---|---|---|---|
| Bacteria | Description | Temperature | 25 kGy | 50 kGy |
| S. epidermidis | Gram Positive | 4° C. | >6.0* | >6.0* |
| E. coli | Gram Negative | 4° C. | >7.1* | >7.1* |
| C. sordellii | Spore Former | 4° C. | 6.3 | >8.0* |
| C. sordellii | Spore Former | −72° C. to −76° C. | 4.5 | >8.0* |

*Maximum reduction detectable in the assay

Having now fully described this invention, it will be understood to those of ordinary skill in the art that the methods of the present invention can be carried out with a wide and equivalent range of conditions, formulations and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

1-122. (canceled)

123: A method for reducing the level of active biological contaminants or pathogens in bone, comprising
contacting the bone with a composition comprising propylene glycol, DMSO, trehalose and mannitol; and
irradiating the bone with gamma radiation,
wherein the total dose of gamma radiation is at least about 20 kGy.

124: The method of claim 123, wherein the bone is at a temperature of about 0° C. to about −196° C. during irradiation.

125: The method of claim 123, wherein the bone is at a temperature of about −50° C. to about −78° C. during irradiation.

126: The method of claim 123, wherein the irradiation is applied at a rate of at least about 0.3 kGy/hour to at least about 30.0 kGy/hour.

127: The method of claim 123, wherein the total dose of gamma irradiation is at least about 45 kGy.

128: The method of claim 123, wherein the total dose of gamma irradiation is at least about 50 kGy.

129: The method of claim 123, wherein the bone is demineralized bone matrix.

130: The method of claim 123, further comprising adjusting or maintaining the pH of the bone prior to irradiation.

131: The method of claim 123, further comprising reducing residual solvent content of the bone prior to irradiation.

132: The method of claim 131, wherein the residual solvent is a non-aqueous solvent.

133: The method of claim 131, wherein the residual solvent is an aqueous solvent.

134: The method of claim 131, wherein the residual solvent content is reduced to about 1% to about 20%.

135: The method of claim 123, further comprising contacting the bone with at least one sensitizer prior to irradiation.

136: The method of claim 123, wherein the active biological contaminants or pathogens are selected from the group consisting of viruses, bacteria, yeasts, molds, fungi, parasites, prions, causative agents of transmissible spongiform encephalopathies and combinations thereof.

137: The method of claim 123 wherein the bone is maintained in an inert atmosphere during irradiation.

138: The method of claim 123 wherein the bone is maintained under vacuum during irradiation.
